(19) United States
(12) Patent Application Publication
(10) Pub. No.: US 2026/0286396 A1
(43) Pub. Date: Sep. 24, 2026

(54) AXL KNOCK OUT CAR T CELLS AND METHODS FOR USE THEREOF

(71) Applicant: Mayo Foundation for Medical Education and Research, Rochester, MN (US)

(72) Inventors: Saad J. Kenderian, Rochester, MN (US); Reona Sakemura, Rochester, MN (US); Elizabeth L. Siegler, Rochester, MN (US)

(21) Appl. No.: 19/472,384

(22) PCT Filed: Apr. 5, 2024
(86) PCT No.: PCT/US2024/023271

§ 371 Date: Oct. 3, 2025

Related U.S. Application Data
(60) Provisional application No. 63/457,334, filed on Apr. 5, 2023.

(51) Int. Cl. C12N 15/90; A61K 40/11; A61K 40/31; A61K 40/42; A61P 35/00; C07K 16/28; C12N 5/0783; C12N 9/22; C12N 15/11

(52) U.S. Cl. CPC C12N 15/907; A61K 40/11; A61K 40/31; A61K 40/4211; A61P 35/00; C07K 16/2803; C12N 5/0636; C12N 9/222; C12N 15/11; C07K 2317/53; C07K 2317/622; C07K 2319/02; C07K 2319/03; C12N 2310/20; C12N 2510/00

(57) ABSTRACT

This document relates to methods and materials involved in treating cancer. For example, methods and materials for using chimeric antigen receptor (CAR) T cells having reduced levels of an Axl polypeptide are provided. Methods and materials for using such CAR T cells in an adoptive cell therapy (e.g., a CAR T cell therapy) to treat a mammal (e.g., a human) having cancer are also provided.

### CROSS-REFERENCE TO RELATED APPLICATIONS

This application claims priority from U.S. Provisional Application Ser. No. 63/457,334, filed Apr. 5, 2023. The disclosure of the prior application is considered part of (and is incorporated by reference in) the disclosure of this application.

### SEQUENCE LISTING

This application contains a Sequence Listing that has been submitted electronically as an XML file named “07039-2187WO1_SL_ST26.XML.” The XML file, created on Apr. 4, 2024, is 27,854 bytes in size. The material in the XML file is hereby incorporated by reference in its entirety.

### TECHNICAL FIELD

This document relates to methods and materials for engineering Axl knock out CAR T cells, and to methods and materials for using the Axl knock out CAR T cells.

### BACKGROUND

Chimeric antigen receptor T (CAR T) cell therapy has evolved as a potent and potentially curative therapy in a subset of patients with hematological malignancies (Maude et al., New Engl J Med., 371:1507-1517, 2014; Lee et al., Lancet, 385:517-528, 2015; Gardner et al., Blood, 128:219, 2016; Turtle et al., J Clin Invest., 126:2123-2138, 2016; Kochenderfer et al., Blood, 119:2709-2720, 2012; Brentjens et al., Blood 118:4817-4828, 2011; and Davila et al., Sci Transl Med., 6: 224ra25-ra25, 2014). However, most patients who are responsive to CAR T treatment relapse or develop resistance within the first year of treatment (Maude et al., N Engl J Med., 378:439-448, 2018; and Park et al., N Engl J Med., 378:449-459, 2018). Additionally, the efficacy of CAR T cell therapy in solid tumors is extremely limited, and objective responses are rarely observed (Wagner et al., Mol Ther., 28:2320-2339, 2020). Mechanisms of CAR T cell failure include intrinsic T cell defects, T cell inhibition by tumor microenvironment, and tumor escape mechanisms (Klebanoff et al., J Clin Invest., 126:318-334, 2016; Sakemura et al., Blood, 2022; and Sakemura et al., Leuk Lymphoma, 2021:1-18, 2021).

Axl is a member of the Tyro3, Axl and proto-oncogene tyrosine-protein kinase Mer (TAM) family of receptor tyrosine kinases (RTKs). TAM RTK polypeptides are made up of an extracellular domain that contains two immunoglobulin-like repeats and two fibronectin type III repeats, a transmembrane domain, and a cytoplasmic protein tyrosine kinase (van der Meer et al., Blood, 123:2460-2469, 2014). Growth arrest-specific protein 6 (Gas6) is the ligand for the TAM family and binds the receptors with different affinities: Axl>Tyro3>Mer (van der Meer et al., supra; and Vouri and Hafizi, Cancer Res., 77:2775-2778, 2017). Axl is expressed on multiple types of immune cells and in a variety of cancers, and has been shown to play multiple roles in regulating tumor cell survival (van der Meer et al., supra; Vouri and Hafizi, supra; Myers et al., Mol Cancer, 18:94, 2019; Seitz et al., J Immunol., 178:5635-5642, 2019; and Zhao et al., Mediators Inflamm., 2017:6848430, 2017).

### SUMMARY

This document is based, at least in part, on the discovery that expression of Axl by immune cells can limit CAR T cell activity, and that inhibiting expression of Axl polypeptides or knocking out Axl polypeptide expression in CAR T cells can enhance anti-tumor activity of the CAR T cells by, for example, overcoming resistance to CAR T cell therapy. As demonstrated herein, resting T cells do not express Axl polypeptides, but activated T cells and CAR T cells express high levels of Axl polypeptides, and activated Th2-CAR T cells and M2-polarized macrophages express particularly high levels of Axl polypeptides. Inhibition of Axl in CAR T cells targeted to CD19 (referred to herein as “CART19” cells) with a high-affinity inhibitor (TP-0903) resulted in selective inhibition of Th2-CAR T cells, reductions in Th2-cytokines, reversal of CAR T cell inhibition, and promotion of CAR T cell effector functions. In addition, inhibition of Axl polypeptides with TP-0903 in vivo in a SCID mouse model improved anti-lymphoma activity and CAR T cell expansion. Thus, Axl inhibition provides a strategy for enhancing CAR T cell functions through two independent but complementary mechanisms: targeting Th2 cells and reversing myeloid-induced CAR T cell inhibition through selective targeting of M2-polarized macrophages.

This document provides T cells (e.g., CAR T cells) having a reduced level of an Axl polypeptide, as well as methods and materials for generating T cells (e.g., CAR T cells) having a reduced level of an Axl polypeptide. For example, a T cell (e.g., a CAR T cell) can be engineered to have reduced Axl polypeptide expression (e.g., for use in adoptive cell therapy). In some cases, a T cell (e.g., a CAR T cell) can be engineered to knock out (KO) a nucleic acid encoding an Axl polypeptide to reduce Axl polypeptide expression in that T cell. In addition, this document provides methods and materials for using T cells (e.g., CAR T cells) having a reduced level of an Axl polypeptide. For example, T cells (e.g., CAR T cells) having a reduced level of an endogenous Axl polypeptide can be administered (e.g., in an adoptive cell therapy) to a mammal (e.g., a human) having cancer to treat the mammal's cancer.

In a first aspect, this document features a method of generating a T cell having a reduced level of an Axl polypeptide. The method can comprise, or consist essentially of, (a) introducing, into a T cell, a nucleic acid encoding a Cas nuclease and a guide RNA, and (b) culturing the T cell under conditions in which the Cas nuclease and the guide RNA are expressed and the Cas nuclease cleaves genomic DNA within the T cell at a nucleotide sequence targeted by the guide RNA, wherein the nucleic acid encoding the guide RNA includes the sequence set forth in SEQ ID NO: 1 or the sequence set forth in any one of the sequence identifiers set forth in TABLE 1. The T cell having a reduced level of an Axl polypeptide can have a disruption in at least one endogenous allele encoding the Axl polypeptide. The T cell having a reduced level of an Axl polypeptide can have a disruption in both endogenous alleles encoding the Axl polypeptide. The T cell can express a reduced level of the Axl polypeptide as compared to a comparable T cell lacking the disruption. The method can further include introducing, into the T cell, a nucleic acid encoding a chimeric antigen receptor. The introducing of the nucleic acid encoding the chimeric antigen receptor can be performed before the introducing of the nucleic acid encoding the Cas nuclease and the guide RNA. The introducing of the nucleic acid encoding the chimeric antigen receptor can be performed after the introducing of the nucleic acid encoding the Cas nuclease and the guide RNA. The chimeric antigen receptor can target a tumor-associated antigen. The tumor-associated antigen can be CD19. The T cell can be from a human.

In another aspect, this document features a T cell (a) having a reduced level of an Axl polypeptide, and (b) containing a nucleic acid encoding a chimeric antigen receptor, wherein the T cell expresses the chimeric antigen receptor, and wherein the T cell was generated by a method that comprises, or consists essentially of, introducing, into the T cell or a precursor thereof, a Cas nuclease and a nucleotide sequence encoding a guide RNA, wherein the nucleotide sequence encoding the guide RNA includes the sequence set forth in SEQ ID NO:1 or the sequence set forth in any one of the sequence identifiers set forth in TABLE 1, such that the Cas nuclease cleaved genomic DNA within the T cell at a nucleotide sequence targeted by the guide RNA. The T cell can have a disruption in at least one endogenous allele encoding the Axl polypeptide. The T cell can have a disruption in both endogenous alleles encoding the Axl polypeptide. The T cell can express a reduced level of the Axl polypeptide as compared to a comparable T cell lacking the disruption. The chimeric antigen receptor can target a tumor-associated antigen. The tumor-associated antigen can be CD19. The T cell can be from a human.

In another aspect, this document features a method for treating a mammal having cancer, where the method can comprise, or consist essentially of, administering to the mammal a composition containing a T cell (a) having a reduced level of an Axl polypeptide, and (b) containing a nucleic acid encoding a chimeric antigen receptor, wherein the T cell expresses the chimeric antigen receptor, and wherein the T cell was generated by a method that includes, or consists essentially of, introducing, into the T cell or a precursor thereof, a Cas nuclease and a nucleotide sequence encoding a guide RNA, wherein the nucleotide sequence encoding the guide RNA includes the sequence set forth in SEQ ID NO:1 or the sequence set forth in any one of the sequence identifiers set forth in TABLE 1, such that the Cas nuclease cleaved genomic DNA within the T cell at a nucleotide sequence targeted by the guide RNA. The T cell can have a disruption in at least one endogenous allele encoding the Axl polypeptide. The T cell can have a disruption in both endogenous alleles encoding the Axl polypeptide. The T cell can express a reduced level of the Axl polypeptide as compared to a comparable T cell lacking the disruption. The chimeric antigen receptor can target a tumor-associated antigen. The tumor-associated antigen can be CD19. The T cell can be from a human. The mammal can be a human. The composition can contain from about 0.1×10⁶ to about 10×10⁶ of the T cells. The cancer can be selected from diffuse large B cell lymphoma (DLBCL), Hodgkin's lymphomas, non-Hodgkin lymphomas, acute lymphoblastic leukemia (ALL), chronic lymphocytic leukemia (CLL), acute myeloid leukemia (AML), germ cell tumors, hepatocellular carcinoma, bowel cancer, lung cancer, breast cancer, ovarian cancer, melanoma, epithelial tumors, brain cancers, multiple myelomas, sarcomas, bone cancers, pancreatic cancers, liver cancers, myeloid neoplasms, prostate cancers, and thyroid cancers.

In another aspect, this document features the use of a composition containing a T cell provided herein to treat a mammal having cancer, where the T cell (a) has a reduced level of an Axl polypeptide, and (b) contains a nucleic acid encoding a chimeric antigen receptor, where the T cell expresses the chimeric antigen receptor, and where the T cell was generated by a method that comprises, or consists essentially of, introducing, into the T cell or a precursor thereof, a Cas nuclease and a nucleotide sequence encoding a guide RNA, where the nucleotide sequence encoding the guide RNA includes the sequence set forth in SEQ ID NO:1 or the sequence set forth in any one of the sequence identifiers set forth in TABLE 1, such that the Cas nuclease cleaved genomic DNA within the T cell at a nucleotide sequence targeted by the guide RNA.

This document also features a composition containing a T cell provided herein for use in the preparation of a medicament to treat a mammal having cancer, where the T cell (a) has a reduced level of an Axl polypeptide, and (b) contains a nucleic acid encoding a chimeric antigen receptor, where the T cell expresses the chimeric antigen receptor, and where the T cell was generated by a method that comprises, or consists essentially of, introducing, into the T cell or a precursor thereof, a Cas nuclease and a nucleotide sequence encoding a guide RNA, wherein the nucleotide sequence encoding the guide RNA includes the sequence set forth in SEQ ID NO:1 or the sequence set forth in any one of the sequence identifiers set forth in TABLE 1, such that the Cas nuclease cleaved genomic DNA within the T cell at a nucleotide sequence targeted by the guide RNA.

In addition, this document features a composition containing a T cell provided herein for use in the treatment of cancer, where the T cell (a) has a reduced level of an Axl polypeptide, and (b) contains a nucleic acid encoding a chimeric antigen receptor, where the T cell expresses the chimeric antigen receptor, and where the T cell was generated by a method that comprises, or consists essentially of, introducing, into the T cell or a precursor thereof, a Cas nuclease and a nucleotide sequence encoding a guide RNA, where the nucleotide sequence encoding the guide RNA includes the sequence set forth in SEQ ID NO: 1 or the sequence set forth in any one of the sequence identifiers set forth in TABLE 1, such that the Cas nuclease cleaved genomic DNA within the T cell at a nucleotide sequence targeted by the guide RNA.

In another aspect, this document features a method of generating a T cell having a reduced level of an Axl polypeptide. The method can comprise, or consist essentially of, (a) introducing, into a T cell, nucleic acid encoding a transcription activator-like effector (TALE) nuclease, wherein the TALE nuclease includes a first monomer and a second monomer, and (b) culturing the T cell under conditions in which the first TALE nuclease monomer and the second TALE nuclease monomer are expressed within the T cell, wherein the first TALE nuclease monomer binds to a first target sequence in genomic DNA of the T cell, wherein the second TALE nuclease monomer binds to a second target sequence in the genomic DNA, and wherein the first and second TALE nuclease monomers dimerize and cleave the genomic DNA of the T cell, wherein the first and second target sequences include a pair of nucleotide sequences set forth in the sequence identifier pairs that are set forth in TABLE 2. The T cell having a reduced level of an Axl polypeptide can have a disruption in at least one endogenous allele encoding the Axl polypeptide. The T cell having a reduced level of an Axl polypeptide can have a disruption in both endogenous alleles encoding the Axl polypeptide. The T cell can express a reduced level of the Axl polypeptide as compared to a comparable T cell lacking the disruption. The method can further include introducing, into the T cell, a nucleic acid encoding a chimeric antigen receptor. The introducing of the nucleic acid encoding the chimeric antigen receptor can be performed before the introducing of the nucleic acid encoding the TALE nuclease. The introducing of the nucleic acid encoding the chimeric antigen receptor can be performed after the introducing of the nucleic acid encoding the TALE nuclease. The chimeric antigen receptor can target a tumor-associated antigen. The tumor-associated antigen can be CD19. The T cell can be from a human.

In still another aspect, this document features a T cell (a) having a reduced level of an Axl polypeptide, and (b) containing nucleic acid encoding a chimeric antigen receptor, wherein the T cell expresses the chimeric antigen receptor, and wherein the T cell was generated by a method that comprises, or consists essentially of, (i) introducing, into the T cell or a precursor thereof, a transcription activator-like effector (TALE) nuclease, wherein the TALE nuclease includes a first monomer and a second monomer, and (ii) culturing the T cell under conditions in which the first TALE nuclease monomer and the second TALE nuclease monomer are expressed within the T cell, wherein the first TALE nuclease monomer binds to a first target sequence in genomic DNA of the T cell, wherein the second TALE nuclease monomer binds to a second target sequence in the genomic DNA, and wherein the first and second TALE nuclease monomers dimerize and cleave the genomic DNA of the T cell, wherein the first and second target sequences include a pair of nucleotide sequences set forth in the sequence identifier pairs that are set forth in TABLE 2. The T cell can have a disruption in at least one endogenous allele encoding the Axl polypeptide. The T cell can have a disruption in both endogenous alleles encoding the Axl polypeptide. The T cell can express a reduced level of the Axl polypeptide as compared to a comparable T cell lacking the disruption. The chimeric antigen receptor can target a tumor-associated antigen. The tumor-associated antigen can be CD19. The T cell can be from a human.

In another aspect, this document features a method for treating a mammal having cancer, where the method comprises, or consists essentially of, administering to the mammal a composition containing a T cell (a) having a reduced level of an Axl polypeptide, and (b) containing nucleic acid encoding a chimeric antigen receptor, wherein the T cell expresses the chimeric antigen receptor, and wherein the T cell was generated by a method that comprises, or consists essentially of, (i) introducing, into the T cell or a precursor thereof, a transcription activator-like effector (TALE) nuclease, wherein the TALE nuclease includes a first monomer and a second monomer, and (ii) culturing the T cell under conditions in which the first TALE nuclease monomer and the second TALE nuclease monomer are expressed within the T cell, wherein the first TALE nuclease monomer binds to a first target sequence in genomic DNA of the T cell, wherein the second TALE nuclease monomer binds to a second target sequence in the genomic DNA, and wherein the first and second TALE nuclease monomers dimerize and cleave the genomic DNA of the T cell, wherein the first and second target sequences include a pair of nucleotide sequences set forth in the sequence identifier pairs that are set forth in TABLE 2. The T cell can have a disruption in at least one endogenous allele encoding the Axl polypeptide. The T cell can have a disruption in both endogenous alleles encoding the Axl polypeptide. The T cell can express a reduced level of the Axl polypeptide as compared to a comparable T cell lacking the disruption. The chimeric antigen receptor can target a tumor-associated antigen. The tumor-associated antigen can be CD19. The T cell can be from a human. The mammal can be a human. The composition can contain from about 0.1×10⁶ to about 10×10⁶ of the T cells. The cancer can be selected from DLBCL, Hodgkin's lymphomas, non-Hodgkin lymphomas, ALL, CLL, AML, germ cell tumors, hepatocellular carcinoma, bowel cancer, lung cancer, breast cancer, ovarian cancer, melanoma, epithelial tumors, brain cancers, multiple myelomas, sarcomas, bone cancers, pancreatic cancers, liver cancers, myeloid neoplasms, prostate cancers, and thyroid cancers.

This document also features the use of a composition containing a T cell provided herein to treat a mammal having cancer, where the T cell (a) has a reduced level of an Axl polypeptide, and (b) contains nucleic acid encoding a chimeric antigen receptor, where the T cell expresses the chimeric antigen receptor, and where the T cell was generated by a method that comprises, or consists essentially of, (i) introducing, into the T cell or a precursor thereof, a transcription activator-like effector (TALE) nuclease, where the TALE nuclease includes a first monomer and a second monomer, and (ii) culturing the T cell under conditions in which the first TALE nuclease monomer and the second TALE nuclease monomer are expressed within the T cell, where the first TALE nuclease monomer binds to a first target sequence in genomic DNA of the T cell, where the second TALE nuclease monomer binds to a second target sequence in the genomic DNA, and where the first and second TALE nuclease monomers dimerize and cleave the genomic DNA of the T cell, and where the first and second target sequences include a pair of nucleotide sequences set forth in the sequence identifier pairs that are set forth in TABLE 2.

In addition, this document features a composition containing a T cell provided herein for use in the preparation of a medicament to treat a mammal having cancer, where the T cell (a) has a reduced level of an Axl polypeptide, and (b) contains nucleic acid encoding a chimeric antigen receptor, where the T cell expresses the chimeric antigen receptor, and where the T cell was generated by a method that comprises, or consists essentially of, (i) introducing, into the T cell or a precursor thereof, a transcription activator-like effector (TALE) nuclease, where the TALE nuclease includes a first monomer and a second monomer, and (ii) culturing the T cell under conditions in which the first TALE nuclease monomer and the second TALE nuclease monomer are expressed within the T cell, where the first TALE nuclease monomer binds to a first target sequence in genomic DNA of the T cell, where the second TALE nuclease monomer binds to a second target sequence in the genomic DNA, and where the first and second TALE nuclease monomers dimerize and cleave the genomic DNA of the T cell, and where the first and second target sequences include a pair of nucleotide sequences set forth in the sequence identifier pairs that are set forth in TABLE 2.

In yet another aspect, this document features a composition containing a T cell provided herein for use in the treatment of cancer, where the T cell (a) has a reduced level of an Axl polypeptide, and (b) contains nucleic acid encoding a chimeric antigen receptor, where the T cell expresses the chimeric antigen receptor, and where the T cell was generated by a method that comprises, or consists essentially of, (i) introducing, into the T cell or a precursor thereof, a transcription activator-like effector (TALE) nuclease, where the TALE nuclease includes a first monomer and a second monomer, and (ii) culturing the T cell under conditions in which the first TALE nuclease monomer and the second TALE nuclease monomer are expressed within the T cell, where the first TALE nuclease monomer binds to a first target sequence in genomic DNA of the T cell, where the second TALE nuclease monomer binds to a second target sequence in the genomic DNA, and where the first and second TALE nuclease monomers dimerize and cleave the genomic DNA of the T cell, and where the first and second target sequences include a pair of nucleotide sequences set forth in the sequence identifier pairs that are set forth in TABLE 2.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C generally illustrate the generation of CART19 cells. FIG. 1A is a schematic showing a CD19-CAR construct containing an anti-CD19 scFv sequence, a CD8 hinge and transmembrane domain, a 4-1BB costimulatory domain, and a CD3 signaling domain. FIG. 1B is a schematic showing the steps in a method for CART19 generation. T cells from normal healthy donors were expanded in vitro with anti-CD3/CD28 DYNABEADS® added on day 0, and T cells were transduced with lentiviral supernatant from 293T cells transfected with a pLV-CAR19 plasmid and two helper plasmids on day 1. The anti-CD3/CD28 DYNABEADS® were removed on day 6, and flow cytometric analysis for CAR expression was performed with goat anti-mouse IgG (H+L) cross-adsorbed secondary antibody (FIG. 1C; UTD, untransduced cells).

FIGS. 2A-2E show that Axl is expressed on activated T cells, CAR T cells, and monocytes. FIG. 2A is a graph plotting Axl expression on stimulated T cells and CAR T cells. Untransduced T cells (UTDs) or CART19 cells were stimulated with 50 ng/ml of phorbol 12-myristate-13-acetate (PMA) and 1.0 μg/mL of ionomycin or the lethally irradiated CD19+ mantle cell lymphoma cell line, JeKo-1 for 24 hours, and were then assessed for Axl surface expression by flow cytometry (*p<0.05, **p<0.01, **** p<0.0001, n.s. not significant, one-way ANOVA, n=3 biological replicates). FIG. 2B includes flow cytometry plots and a graph showing that activated Th2 cells express higher levels of Axl than Th1 cells. Stimulated T cells were further assessed for helper T cell (Th) phenotypes. Th1 were defined as CD4⁺CCR6⁻CXCR3⁺CCR4⁻ and Th2 cells were defined as CD4⁺CCR6⁻CXCR3⁻CCR4⁺. Axl expression as assessed by flow cytometry is plotted in the graph at right (*p<0.0005, t-test, n=3 biological replicates).

FIG. 2C includes images of western blot analysis of rested UTD or CART19 cells after their preparation, assessed for Axl. FIG. 2D is a flow cytometry histogram showing that that monocytes express high levels of Axl on their cell surface. Monocytes were isolated by negative selection using the human classical monocyte isolation kit, and Axl expression was assessed by flow cytometry. FIG. 2E includes flow cytometry histograms and a graph showing that M2-polarized macrophages express higher Axl levels compared to M1-polarized macrophages. M1 or M2 induction was performed by addition of LPS and IFN-γ or IL-4, respectively. Surface Axl expression on M1 and M2 was assessed by flow cytometry (**p<0.01, t-test, n=3 biological replicates). Data are plotted as means±SEM.

FIGS. 3A to 3G show Axl expression on T cells and monocytes. FIG. 3A includes flow plots and histograms showing gating strategies for CD8⁺ and helper T (Th) cells. In order to define the negative fractions for CCR4, CXCR3, and Axl, fluorescence minus one (FMO) was used. FIG. 3B is a graph plotting percent Axl on Th cells, and showing that activated Th2 cells express higher levels of Axl than Th1 cells. Stimulated T cells were further assessed for Th phenotypes. Th1 and Th2 cells were defined by CD4⁺CCR6⁻CXCR3⁺CCR4⁻ and CD4⁺CCR6⁻ CXCR3⁻CCR4+, respectively. Axl expression was assessed by flow cytometry (*p<0.05, t-test, n=3 biological replicates). FIG. 3C includes graphs plotting percent Axl expression, and showing that activated Th2 cells increased their surface Axl expression while Th1 showed no difference. CART19 cells were stimulated with either CD19⁺ JeKo-1 cells or PMA/Ionomycin for 24 hours and surface expression of Axl was determined with flow cytometry. FIG. 3D includes flow plots and histograms showing gating strategies for CD14⁺ monocytes. Axl on monocytes was defined with FMO. FIG. 3E is a histogram plotting Axl expression on JeKo-1 cells. FMO was used to define the positive fraction of Axl on monocytes. FIG. 3F includes flow plots showing a gating strategy for assessing live CD14⁺ monocytes. FIG. 3G is a graph plotting surface Axl expression on M1 or M2 type macrophages. ***p<0.001, t-test n=3 biological replicates.

FIGS. 4A-4E show that Axl inhibition with TP-0903 selectively downregulates monocytes and modulates phenotypes of naïve T cells and CART19 cells. FIG. 4A includes graphs showing that Axl inhibition does not interfere T cell degranulation or cytokine production, but preferentially reduces Th2 type cytokines. Naïve T cells were stimulated with 50 ng/ml of PMA and 1 μg/mL of Ionomycin for 4 hours in the presence of increasing doses of TP-0903 (10 to 30 nM). Flow cytometric analysis revealed similar activation of T cells in the presence of TP-0903, as shown by CD107a degranulation and intracytoplasmic cytokine production (IL-2 and IFN-γ). However, there was a significant inhibition of IL-4 and IL-13 with TP-0903 treatment. FIG. 4B includes flow cytometry plots and a graph showing that Axl inhibition polarizes T cells to Th1 cells. Naïve T cells were stimulated with 5 ng/ml of PMA and 0.1 μg/ml of Ionomycin for 3 days in the presence of TP-0903. Flow cytometric analysis revealed a significant upregulation of CD4⁺CXCR3⁺CCR4⁺ T cells (**p<0.01, t-test). FIG. 4C includes graphs showing that Axl inhibition improves antigen specific CART19 cell proliferation. CART19 cells were co-cultured with the CD19 mantle cell lymphoma cell line, JeKo-1, in the absence or presence of TP-0903 (10 nM) for 5 days. TP-0903 improved CART19 cell expansion. This was clearly observed for CD8 T cells (center graph). FIG. 4D includes flow cytometry plots and graphs showing that Axl inhibition polarizes CART19 cells to Th1 phenotype. CART19 cells were co-cultured with JeKo-1 in the presence of TP-0903 (10 nM) for 5 days, and chemokine receptors were stained. Similar to naïve T cells, flow cytometric analysis revealed upregulation of Th1 cells and CD4 CXCR3 CCR4 fraction, and reduction of Th2 cells (**p<0.005, t-test). FIG. 4E is a series of graphs showing that Axl inhibition preferentially reduces Th2 type cytokines and myeloid cell related cytokines. CART19 cells were co-cultured with JeKo-1 cells for 3 days and supernatants were analyzed for human cytokines and chemokines (38-multiplex, Millipore). The ability of effector cytokine production was maintained in the presence of TP-0903, while Th2-related cytokines such as IL-4 and IL-5 were significantly downregulated. Cytokines known as cytokine related syndrome, such as IL-6, IL-10, and MIP1-β, were also remarkably downregulated in the presence of TP-0903. Data are plotted as means±SEM.

FIGS. 5A-5D are graphs plotting the results of cytotoxicity assays. FIG. 5A is a graph plotting percent killing in a TP-0903 cytotoxicity assay against the CD19⁺ cell line, JeKo-1. Luciferase⁺ JeKo-1 cells were treated with increasing doses of TP-0903 (10-65 nM) or DMSO control. At 48 hours, cell killing was assessed by luminescence relative to controls. ****p<0.0001, one-way ANOVA. FIG. 5B is a graph plotting the results of a CART19 cytotoxicity assay against CD19⁺ JeKo-1 cells in the presence of TP-0903. CART19 were co-cultured at different effector-to-target ratios (E: T) with luciferase⁺ JeKo-1 cells. At 48 hours, cell killing was assessed by luminescence relative to controls. ****p<0.0001, one-way ANOVA. FIG. 5C is a graph plotting TP-0903 cytotoxicity assay against leukemic B cells derived from CLL 24 patients. Leukemic B cells were treated with increasing doses of TP-0903 (10-65 nM) or DMSO control. At 48 hours, cytotoxicity was determined by flow cytometry. ***p=0.0001, 26 one-way ANOVA. FIG. 5D is a graph plotting CART19 cytotoxicity assay against leukemic B cells derived from CLL patients. CART19 were co-cultured at different E:T ratios with leukemic B cells derived from CLL patients. 48 hours after the co-culture, cytotoxicity was determined by flow 29 cytometry. ****p<0.0001, one-way ANOVA.

FIGS. 6A and 6B illustrate the generation of AxlKO CART19 cells via lentiviral transduction of T cells. FIG. 6A is a schematic of a CRISPR lentivirus backbone, showing a sequence (SEQ ID NO:1) encoding a gRNA and the complement (SEQ ID NO: 2) of the sequence. FIG. 6B is a schematic of an experimental design in which AxlKO CART19 cells were generated via transduction of a lentiviral vector carrying Cas9 and gRNA under the control of a U6 promoter.

FIG. 7A includes a histogram and a graph plotting Axl expression, showing that Axl was efficiently knocked out from CART19 cells. *p<0.05, 37 t-test, n=3. FIG. 7B includes a graph and flow plots showing that knocking out Axl with CRISPR did not impact CAR T Cell expansion during CAR T cell manufacturing. FIG. 7C includes flow plots showing gating strategies for helper T (Th) cells and a graph plotting Axl expression. In order to define the negative fractions for CCR4 and CXCR3 fluorescence, minus one (FMO) was used. FIG. 7D includes representative flow plots and graphs for Axl^{wt} CART19 and Axl^{ko} CART19, showing that Axl^{ko} CART19 cells significantly increased the CXCR3⁻CCR4⁺ population and decreased the Th2 (CCR4⁺CXCR3⁻) population. FIG. 7E includes graphs plotting percent killing and T cell numbers, showing that Axl^{ko} CART19 significantly decreased the Th2 population and increased the CXCR3⁺CCR4⁺ fraction. **p<0.01, 41 t-test, n=3 biological replicates.

FIG. 8 is a graph plotting the results of CRISPR/Cas9 editing of Axl in CART19 cells. A representative TIDE sequence verified allelic modification frequency in Axl^{ko} CART19 cells. DNA was isolated from Axl^{ko} CART19 cells, followed by PCR and Sanger sequencing of the resulting PCR product. Sequencing results were run through TIDE software to calculate the efficacy of knockout based on number of insertions and deletions on the targeted gene region.

FIGS. 9A-9F show that CART19 and TP-0903 combination therapy had anti-tumor and survival effects superior to those of CART19 monotherapy in a relapse model. FIG. 9A includes images of mice in a JeKo-1 relapse model. 1.0×10⁶ luciferase-positive JeKo-1 cells were injected into NSG mice via the tail vein. Two weeks after injection, the tumor burden was analyzed by bioluminescence imaging. Mice were randomized into vehicle (n=4), 20 mg/kg TP-0903 (p.o.) (n=4), 0.5×10⁶ CART19 (i.v.) (n=5), and 20 mg/kg TP-0903 with 0.5×10⁶ CART19 (n=5) groups. FIG. 9B is a graph providing a summary of the bioluminescence signal as a measurement of tumor growth after JeKo-1 cell inoculation and administration of CART19 with or without TP-0903. The combinatory group showed better tumor control at day 21 and day 28 (**p<0.005 and ****p<0.0001, two-way ANOVA). FIG. 9C is a Kaplan-Meier survival plot showing survival curves indicating significantly increased median survival of the combinatory group compared with the CART19 monotherapy group (**p<0.005, log-rank test). FIG. 9D is a graph plotting the results of peripheral blood analysis at day 17 of CART19 expansion in response to a combination of TP-0903 and CART19 cells as compared to CART19 alone. The combinatory group showed significant CART19 cell expansion (* p<0.05, t-test; monotherapy group n=6 and combination therapy group n=8). Data are plotted as means±SEM. FIGS. 9E and 9F include a flow cytometry plot and graphs showing CART19 cell phenotypes in splenocytes harvested from satellite mice at day 17 of CART19 cell treatment. The flow cytometric analysis demonstrated that the combination therapy group showed significant Th1 polarization of CART19 cells as compared to the monotherapy group (FIG. 9F; *p<0.05, t-test, both groups n=3).

FIGS. 10A-10E show that Axl inhibition with TP-0903 preferentially targets monocytes and overcomes monocyte-induced CAR T cell suppression. FIG. 10A includes graphs plotting monocyte, T cell, and B cell numbers, and showing that Axl inhibition with TP-0903 selectively inhibits monocytes. Peripheral blood mononuclear cells derived from healthy donors were treated with increasing doses of TP-0903 (10-65 nM) for 24 hours, and the absolute number of cells was assessed by CountBright bead quantification with flow cytometry (n.s. not significant, **p<0.01, ****p<0.001, one-way ANOVA, n=3 biological replicates). FIG. 10B is a graph plotting CART19 cell numbers, showing that monocytes inhibit CART19 cell expansion but TP-0903 overcomes the monocyte-induced CART19 suppression. CART19, JeKo-1, and monocytes were co-cultured for 5 days and analyzed for the absolute number of CD3⁺ T cells by CountBright bead quantification (*p<0.05, t-test, n=3 biological replicates). FIG. 10C includes graphs showing that Axl inhibition with TP-0903 reduces myeloid cytokines. Supernatants harvested from solid tumor patient peripheral blood-derived T cells were analyzed with 38-mulitplex (Millipore) (*p<0.05, **p<0.01, ***p<0.001, ****p<0.0001, one-way ANOVA, n=3 biological replicates). FIG. 10D includes flow cytometry histograms showing that Axl inhibition prevents M2 polarization. CD14⁺ cells were stained for CD206 and CD163 (markers characteristic for the M2 population) and analyzed via flow cytometry. FIG. 10E is a graph plotting M2 macrophage numbers, showing that Axl inhibition selectively reduces M2-type macrophages. CART19, JeKo-1, and M2-type macrophages were co-cultured for 5 days and were analyzed for the absolute number of CD14⁺ cells by flow cytometry (**p<0.01, t-test, n=3 biological replicates). Data are plotted as means±SEM.

FIG. 11 includes a pair of flow plots showing flow cytometric analysis before and after monocyte isolation. CD14⁺ monocytes were isolated from healthy donor derived PBMCs using a classical monocyte isolation kit (negative selection). The purity of isolated cell populations was controlled by flow cytometry (routinely>95%).

FIGS. 12A-12D show CART19 transcriptomic changes following Axl inhibition. FIG. 12A contains representative images from Western blot analysis showing no significant changes in Axl downstream signaling pathways following low doses of TP-0903. CART19 cells were incubated with lethally irradiated JeKo-1 cells in the presence of TP-0903 (10-30 nM) at a 1:3 ratio for 24 hours. After the incubation, JeKo-1 cells were depleted with anti-CD4 and -CD8 micro beads prior to the assay. FIG. 12B is a volcano plot generated from RNA-seq analysis of untreated CART19 cells and CART19 cells treated with 30 nM TP-0903, which showed distinct gene expression patterns. FIG. 12C is a heatmap showing differential expression of nearly 119 transcripts, including 85 upregulated genes and 34 downregulated genes, in TP-0903-treated CART19 cells compared to untreated CART19 cells. CART19 cells from three biological replicates were thawed and stimulated with irradiated JeKo-1 (120 Gy) for 5 days. Each sample was treated with either 30 nM TP-0903 (treated condition) or DMSO (untreated condition). RNA-seq was performed on an Illumina HTSeq 4000. FIG. 12D indicates gene ontology for significantly upregulated genes in CART19 treated with TP-0903. Biological processes that overlap with the significantly differentially upregulated genes were identified using Enrichr. Differential expression analysis between TP-0903 treated or untreated CART19 confirmed upregulation of genes related to tumor infiltrating T lymphocytes, upregulating cytotoxic T cell functions, or suppressing regulatory T cell functions (COL5A1, PDE4C, and HTRA1). TP-0903-treated CART19 also demonstrated downregulation of genes related to promoting regulatory T cells (ALDHIL2 and PHGDH).

FIG. 13 includes a pair of flow plots showing flow cytometric analysis before and after CART19 isolation. CART19 cells were co-cultured with CD19⁺ JeKo-1 cells for 24 hours at a 1:1 ratio. After the co-culture, CART19 cells were isolated using CD4⁺ and CD8⁺ microbeads (routinely>95%).

### DETAILED DESCRIPTION

This document provides methods and materials for generating T cells (e.g., CAR T cells) having a reduced level of an Axl polypeptide (e.g., CAR T cells having a genetic knock out of nucleic acid encoding an Axl polypeptide, such as Axl KO CAR T cells). In some cases, a T cell (e.g., a CAR T cell, which also can be referred to as a CAR⁺ T cell) can be engineered to knock out a nucleic acid encoding an Axl polypeptide to reduce Axl polypeptide expression in that T cell (e.g., as compared to a comparable T cell that is not engineered to knock out a nucleic acid encoding an Axl polypeptide). A T cell that is engineered to knock out a nucleic acid encoding an Axl polypeptide can also be referred to herein as an Axl KO T cell, an Axl^{k/o} T cell, or an Axl^{KO} T cell.

The term “reduced level” as used herein with respect to a level of an Axl polypeptide refers to any level that is lower than a reference level of an Axl polypeptide. The term “reference level” as used herein with respect to an Axl polypeptide refers to the level of that polypeptide typically observed in control T cells from one or more mammals (e.g., humans) not engineered to have a reduced level of an Axl polypeptide as described herein. Control T cells can include, without limitation, T cells that are wild-type T cells. In some cases, a reduced level of an Axl polypeptide can be an undetectable level of that polypeptide. In some cases, a reduced level of an Axl polypeptide can be an eliminated level of that polypeptide.

In some cases, a T cell having (e.g., engineered to have) a reduced level of an Axl polypeptide can have enhanced anti-tumor activity. In such cases, resistance to CAR T cell therapy can be less likely to occur after administration of the CAR T cell to a mammal (e.g., as compared to a CAR T cell that is not engineered to have a reduced level of an Axl polypeptide). For example, reducing a level of an Axl polypeptide in a CAR T cell can be effective to increase the anti-tumor activity of the CAR T cell by, for example, 10, 20, 30, 40, 50, 60, 70, 80, 90, 95, or more percent as compared to a CAR T cell that is not engineered to have a reduced level of an Axl polypeptide. In some cases, a T cell having (e.g., engineered to have) a reduced level of an Axl polypeptide can have enhanced CAR T cell function such as improved antitumor activity, improved proliferation, and/or improved cell killing (e.g., improved killing of tumor cells) (e.g., as compared to a CAR T cell that is not engineered to have a reduced level of an Axl polypeptide).

Any appropriate method can be used to assess one or more functions of T cells (e.g., T cells having a reduced level of an Axl polypeptide). Examples of methods that can be used to evaluate T cell (e.g., CAR T cell) functions include, without limitation, the treatment of in vivo tumor models, proliferation assays (to determine absolute T cell numbers following antigen-specific stimulation), cytotoxicity assays (e.g., to evaluate whether or not T cells (e.g., CAR T cells) are effective at killing target cells).

A T cell having (e.g., engineered to have) a reduced level of an Axl polypeptide can be any appropriate T cell. A T cell can be a naïve T cell. Examples of T cells that can be engineered to have a reduced level of an Axl polypeptide as described herein include, without limitation, cytotoxic T cells, helper T cells, CD4⁺ T cells, and CD8⁺ T cells. For example, a T cell that can be engineered to have a reduced level of an Axl polypeptide as described herein can be a CAR T cell. In some cases, one or more T cells designed to have a reduced level of an Axl polypeptide can be T cells that were obtained from a mammal (e.g., a mammal having cancer) that is to be treated with those T cells designed to have a reduced level of an Axl polypeptide. For example, T cells can be obtained from a mammal to be treated with the materials and method described herein.

A T cell having (e.g., engineered to have) a reduced level of an Axl polypeptide can be generated using any appropriate method. In some cases, a T cell (e.g., a CAR T cell) can be treated with one or more inhibitors to reduce polypeptide expression in that T cell (e.g., as compared to a T cell that was not treated with the one or more inhibitors). For example, a T cell (e.g., a CAR T cell) can be treated with one or more Axl polypeptide inhibitors to reduce a level of Axl polypeptides in that T cell (e.g., as compared to a T cell that was not treated with the one or more Axl polypeptide inhibitors).

In some cases, T cells (e.g., CAR T cells) and one or more Axl polypeptide inhibitors can be administered to a mammal (e.g., a human) having cancer to reduce Axl polypeptide expression within the mammal (e.g., to generate CAR T cells having enhanced anti-tumor activity within the mammal). For example, T cells (e.g., CAR T cells) and one or more Axl polypeptide inhibitors can be administered to a mammal (e.g., a human) having cancer (e.g., to generate CAR T cells having enhanced anti-tumor activity within the mammal). In some cases, T cells (e.g., CAR T cells) having a reduced level of an Axl polypeptide (e.g., Axl KO CAR T cells) and one or more Axl polypeptide inhibitors can be administered to a mammal (e.g., a human) having cancer to reduce Axl polypeptide expression within the mammal (e.g., to generate CAR T cells having enhanced anti-tumor activity within the mammal).

An Axl polypeptide inhibitor can be any appropriate Axl polypeptide inhibitor. An Axl polypeptide inhibitor can be an inhibitor of Axl polypeptide expression or an inhibitor of Axl polypeptide activity. In some cases, an Axl inhibitor can inhibit one or more polypeptides that can regulate production of an Axl polypeptide (e.g., one or more polypeptides that are upstream regulators of an Axl polypeptide). Examples of compounds that can inhibit Axl polypeptide activity include, without limitation, antibodies (e.g., neutralizing antibodies) that target (e.g., target and bind) to an Axl polypeptide, and small molecules that target (e.g., target and bind) to an Axl polypeptide. Examples of compounds that can inhibit Axl polypeptide expression include, without limitation, nucleic acid molecules designed to induce RNA interference of polypeptide expression of an Axl polypeptide (e.g., a siRNA molecule or a shRNA molecule), antisense molecules, and miRNAs. Examples of Axl polypeptide inhibitors that can be contacted with a T cell (e.g., CAR T cell) to reduce a level of an Axl polypeptide within the T cell include, without limitation, TP-0903 (Dubermatinib), which is available commercially (e.g., from Tolero Pharmaceuticals, Inc.; Lehi, Utah).

In some cases, a T cell (e.g., a CAR T cell) can be engineered to KO a nucleic acid encoding an Axl polypeptide to reduce Axl polypeptide expression in that T cell. For example, at least one endogenous allele of a nucleic acid encoding an Axl polypeptide can be disrupted (e.g., knocked out) to generate a T cell (e.g., a CAR T cell) having a reduced level of an Axl polypeptide. In some cases, both endogenous alleles of a nucleic acid encoding an Axl polypeptide can be disrupted (e.g., knocked out) to generate a T cell (e.g., a CAR T cell) having a reduced level or no level of an Axl polypeptide.

In some cases, when a T cell (e.g., a CAR T cell) can be engineered to KO a nucleic acid encoding an Axl polypeptide to reduce Axl polypeptide expression in that T cell, any appropriate method can be used to KO the nucleic acid encoding the Axl polypeptide. Examples of techniques that can be used to knock out a nucleic acid encoding an Axl polypeptide include, without limitation, gene editing, homologous recombination, non-homologous end joining, microhomology end joining, and base editing. For example, gene editing (e.g., with engineered nucleases) can be used to knock out a nucleic acid encoding an Axl polypeptide. Nucleases useful for genome editing include, without limitation, CRISPR-associated (Cas) nucleases, zinc finger nucleases (ZFNs), transcription activator-like effector (TALE) nucleases, and homing endonucleases (HE; also referred to as meganucleases).

In some cases, a clustered regularly interspaced short palindromic repeat (CRISPR)/Cas system can be used (e.g., can be introduced into one or more T cells) to KO a nucleic acid encoding an Axl polypeptide. A CRISPR/Cas system used to KO a nucleic acid can include a guide RNA (gRNA) that is complementary to the target nucleic acid (e.g., a nucleic acid encoding an Axl polypeptide). The nucleic acid sequence targeted by the guide RNA can be followed by a protospacer adjacent motif (PAM), which canonically has the sequence 5′-NGG-3′. An example of a nucleotide sequence encoding a gRNA that is specifically targeted to a nucleic acid encoding an Axl polypeptide is set forth in SEQ ID NO:1 (TTCGGTGTCAGCTCCAGGTT). In some cases, a gRNA can be designed based on a sequence of a nucleic acid encoding an Axl polypeptide. Examples of nucleic acids encoding an Axl polypeptide sequence include, without limitation, those set forth in National Center for Biotechnology Information (NCBI) accession no. NM_021913.5, accession no. NM_001699.6, and accession no. NM_001278599.2. For example, a gRNA specific to a nucleic acid encoding an Axl polypeptide can be as described elsewhere (see, e.g., Axelrod et al., Mol Cancer Res., 17:356-369, 2019). Examples of other gRNA sequences that can be used in the methods provided herein are set forth in TABLE 1.

TABLE 1
Nucleotide sequences encoding
gRNAs targeted to human Axl

| SEQ ID NO: | gRNA encoding sequence |
|---|---|
| 4 | CACCCCTTATCACATCCGCG |
| 5 | ACCGATGAGCCTCATGACGT |
| 6 | ATGCCACGCGGATGTGATAA |
| 7 | CGCGTAGCACTAATGTTCTC |
| 8 | GACTGTAGGACTTGCGCACG |
| 9 | GTTCTCCACGCCCGGATATG |
| 10 | TTGACATAGAGGATTTCGTC |
| 11 | CGCCAGCCGGTACCCTAACA |
| 12 | GCATCTTGGCGATACGTCCC |
| 13 | TCTTGGCGATACGTCCCTGG |
| 14 | TGCGCAAGTCCTACAGTCGT |
| 15 | ACATTAGTGCTACGCGGAAT |
| 16 | AATGGGGACTACTACCGCCA |
| 17 | GAGATTCATACACCGGGACC |
| 18 | CGAAAGAAGGAGACCCGTTA |
| 19 | CCTTGTCCACCGGCGAAAGA |

A CRISPR/Cas system used to KO a nucleic acid encoding an Axl polypeptide can include any appropriate Cas nuclease. Examples of Cas nucleases include, without limitation, Cas1, Cas2, Cas3, Cas9, Cas10, Cpf1, Cas12a, and ErCas12a. In some cases, a Cas component of a CRISPR/Cas system designed to KO a nucleic acid encoding an Axl polypeptide can be a Cas9 nuclease. An exemplary Cas9 nuclease can have the amino acid sequence set forth in Example 2. For example, the Cas9 nuclease of a CRISPR/Cas9 system described herein can be a Streptococcus pyogenes Cas9 nuclease (see, e.g., Cox et al., Leukemia, 36 (6): 1635-1645 (2022)).

Components of a CRISPR/Cas system (e.g., a gRNA and a Cas nuclease) used to KO a nucleic acid encoding an Axl polypeptide can be introduced into one or more T cells (e.g., CAR T cells) in any appropriate format. In some cases, a component of a CRISPR/Cas system can be introduced into one or more T cells as a nucleic acid encoding a gRNA and/or a nucleic acid encoding a Cas nuclease. For example, a nucleic acid encoding at least one gRNA and a nucleic acid encoding at least one Cas nuclease (e.g., a Cas9 nuclease) can be introduced into one or more T cells. In some cases, a component of a CRISPR/Cas system can be introduced into one or more T cells as a gRNA and/or as a Cas nuclease. For example, at least one gRNA and at least one Cas nuclease (e.g., a Cas9 nuclease) can be introduced into one or more T cells.

In some cases, a ZFN system can be used (e.g., can be introduced into one or more T cells) to KO a nucleic acid encoding an Axl polypeptide. A ZFN system used to KO a nucleic acid can include a polypeptide including (a) a DNA-binding domain (e.g., zinc fingers) that is complementary to a target nucleic acid (e.g., a nucleic acid encoding an Axl polypeptide), and (b) a nuclease domain (e.g., a nuclease domain that can created double-strand breaks). A ZFN system used to KO a nucleic acid encoding an Axl polypeptide can include any appropriate nuclease domain. In some cases, a nuclease domain of a ZFN system designed to KO a nucleic acid encoding an Axl polypeptide can be a Fok1 nuclease domain.

In some cases, a TALE nuclease system can be used (e.g., can be introduced into one or more T cells) to KO a nucleic acid encoding an Axl polypeptide. A TALE nuclease system used to KO a nucleic acid can include a polypeptide including (a) a transcription activator-like effector (TALE) DNA-binding domain directing the nuclease to a target nucleic acid (e.g., a nucleic acid encoding an Axl polypeptide), and (b) a nuclease domain (e.g., a nuclease domain that can create double-strand breaks). A TALE nuclease system used to KO a nucleic acid encoding an Axl polypeptide can include any appropriate nuclease. In some cases, a nuclease can be a non-specific nuclease. In some cases, a nuclease of a TALE nuclease system designed to KO a nucleic acid encoding an Axl polypeptide can be a FokI nuclease. In some cases, a nuclease can function as a dimer across a bipartite recognition site with a spacer, such that two TALE domains are each fused to a catalytic domain of a nuclease (e.g., FokI). In such cases, the DNA recognition sites for the two TALE nuclease monomer can be separated by a spacer sequence, and binding of each TALE nuclease monomer to its recognition site can allow the nuclease to dimerize and create a double-strand break within the spacer (see, e.g., Moscou and Bogdanove (2009) Science 326:1501). Examples of TALE nuclease target sequence pairs that can be used to KO expression of a human Axl polypeptide are set forth in TABLE 2.

TABLE 2
TALE nuclease target sequences

| SEQ | First TALEN | SEQ | Second TALEN |
|---|---|---|---|
| ID NO: | sequence of pair | ID NO: | sequence of pair |
| 20 | GGCTGTGCTGTCAGAC | 21 | GGCACTGATATAAGCA |
| | GAT | | AGGTCC |
| 22 | CTCTCCTCAGGCTGTG | 23 | GGCACTGATATAAGCA |
| | CTGT | | AGGTCC |
| 24 | CTGTCAGACGATGGGA | 25 | GGCACTGATATAAGCA |
| | TGG | | AGGTCC |
| 26 | AGGCTGTGCTGTCAGA | 27 | GGCACTGATATAAGCA |
| | CGAT | | AGGTCC |
| 28 | GTGCTGTCAGACGATG | 29 | GGCACTGATATAAGCA |
| | GGAT | | AGGTCC |

Components of a gene-editing system (e.g., a CRISPR/Cas system) used to KO a nucleic acid encoding an Axl polypeptide can be introduced into one or more T cells (e.g., CAR T cells) using any appropriate method. A method of introducing components of a gene-editing system into a T cell can be a physical method. A method of introducing components of a gene-editing system into a T cell can be a chemical method. A method of introducing components of a gene-editing system into a T cell can be a particle-based method. Examples of methods that can be used to introduce components of a gene-editing system into one or more T cells include, without limitation, electroporation, transfection (e.g., lipofection), transduction (e.g., viral vector mediated transduction), microinjection, nucleofection, CELL SQUEEZE®, and microfluidics.

A T cell having (e.g., engineered to have) a reduced level of an Axl polypeptide can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, a T cell can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor expressed in a T cell having a reduced level of an Axl polypeptide as described herein include, without limitation, cluster of differentiation 19 (CD19), mucin 1 (MUC-1), human epidermal growth factor receptor 2 (HER-2), estrogen receptor (ER), epidermal growth factor receptor (EGFR), alphafetoprotein (AFP), carcinoembryonic antigen (CEA), CA-125, epithelial tumor antigen (ETA), melanoma-associated antigen (MAGE), CD33, CD123, CLL-1, E-Cadherin, folate receptor alpha, folate receptor beta, IL13R, EGFRviii, CD22, CD20, kappa light chain, lambda light chain, desmopressin, CD44v, CD45, CD30, CD5, CD7, CD2, CD38, BCMA, CD138, FAP, CS-1, C-met, TSHR, and EphA3. For example, a T cell having a reduced level of an Axl polypeptide can be designed to express an antigen receptor targeting CD19.

In some cases, a CAR can be designed to include a single chain antibody (e.g., a scFv) targeting a tumor antigen. For example, a CAR can be designed to include a single chain antibody as set forth in TABLE 3. In some cases, a CAR for creating a CAR T cell having a reduced level of an Axl polypeptide can be designed to include a single chain antibody sequence having the three heavy chain complementarity-determining regions (CDRs) (e.g., HC-CDR1, HC-CDR2, and HC-CDR3) and the three light chain CDRs (e.g., LC-CDR1, LC-CDR2, and LC-CDR3) of a scFv antibody set forth in TABLE 3.

TABLE 3
Exemplary CARs for targeting tumor antigens

| Antigen | scFv antibody name | Reference(s) |
|---|---|---|
| CD19 | FMC63 | Milone et al., Mol. Ther., 17(8): 1453-64 (2009) |
| | | (PMID: 26330164) |
| | MOR208 | Kellner et al., Leukemia, 27(7): 1595-8 (2013) |
| | | (PMID: 23277329) |
| | Humanized scFv | International Patent Application Publication No. |
| | | WO2015157252 |
| | 4G7 | EP Patent No. EP2997141 |
| | Low affinity scFv | Chinese Patent No. CN107406517 |
| | FMC63 CAR KHYG-1 | Kang et al., Int. J. Mol. Sci., 21(23): 9163 (2020) |
| | and 4G7 CAR KHYG-1 | |
| MUC-1 | 5E5 | Posey et al., Immunity, 45(5): 947-948 (2016) |
| | | (PMID: 27851918) |
| HER-2 | 4D5 | Ohnishi et al., Br. J. Cancer, 71(5): 969-73 (1995) |
| | | (PMID: 7734322) |
| | | Forsberg et al., Cancer Res., 79(5): 899-904 (2019) |
| | | (PMID: 30622115) |
| | | Nellan et al., J. Immunother. Cancer, 6(1): 30 (2018) |
| | | (PMID: 29712574) |
| | | Priceman et al., Clin Cancer Res., 24(1): 95-105 |
| | | (2018) (PMID: 29061641) |
| | FRP5 | Bielamowics et al., Neuro. Oncol., 20(4): 506-518 |
| | | (2018) (PMID: 29016929) |
| | K70H-28z, K70L-28z, | Zhou et al., Front. Cell Develop. Biol., 10: (2022). |
| | KSAbH-28z and KSAbL- | |
| | 28z | |
| EGFR | M27 | Jiang et al., Cancer Immunol. Res., 6(11): 1314-1326 |
| | | (2018) (PMID: 30201736) |
| | Cetuximab | Caruso et al., Cancer Res., 75(17): 3505-18 (2015) |
| | | (PMID: 26330164) |
| Folate | C4 based | Ao et al., J. Immunother., 42(8): 284-296 (2019) |
| receptor | | (PMID: 31261167) |
| alpha | MOv19 | Song et al., J. Hematol. Oncol., 9(1): 56 (2016) |
| | | (PMID: 27439908) |
| Mesothelin | SS1 | Haas et al., Mol. Ther., S1525-0016(19)30328-4 |
| | | (2019) (PMID: 31420241) |
| | M clone | Adusumilli et al., Sci. Transl. Med., |
| | | 6(261): 261ra151 (2014) (PMID: 25378643) |
| AFP | ET1402L1 | Liu et al., Clin. Cancer Res., 23(2): 478-488 (2017) |
| | | (PMID: 27535982) |
| CEA | Anti-CEA scFv | Chi et al., Cancer Med., 8(10): 4753-4765 (2019) |
| | | (PMID: 31237116) |
| | CEACAM5 | Thistlethwaite et al., Cancer Immunol. Immunother., |
| | | 66(11): 1425-1436 (2017) (PMID: 28660319) |
| | hMN14 | Katz et al., Clin. Cancer Res., 21(14): 3149-59 |
| | | (2015) (PMID: 25850950) |
| CD123 | 22172, 22176 | Gill et al., Blood, 123(15): 2343-54 (2014) (PMID: |
| | | 24596416) |
| | Humanized scFv | US Patent Application Publication No. |
| | | 2016/0068601 |
| | Humanized scFv | EP Patent No. EP2968415 |
| | Tagrazofusp based | Pemmaraju et al. N. Engl. J. Med., 380(17): |
| | | 1628-1637 (2019) (PMID: 31018069) |
| CD33 | MY96 | Kenderian et al., Leukemia, 29(8): 1637-47 (2015) |
| | | (PMID: 25721896) |
| | Humanized scFv | International Patent Application Publication No. |
| | | WO2016014576 |
| CLEC12A | Humanized scFv | US Patent Application Publication No. |
| | | US20160051651 |
| | CLL1 scFv | International Patent Application Publication No. |
| | | WO2016120219 |
| CD22 | M0971 | Fry et al., Nat. Med., 24(1): 20-28 (2018) (PMID: |
| | | 29155426) |
| | Humanized scFv clones | International Patent Application Publication No. |
| | | WO/2016/164731 |
| | Inotuzumab based | Kantarjian et al., N. Engl. J. Med., 375(8): 740-53 |
| | | (2016) (PMID: 27292104) |
| | Moxetumomab based | Kreitman et al., Leukemia, 32(8): 1768-1777 (2018) |
| | | (PMID: 30030507) |
| CD20 | Rituximab | Zhang et al., Signal Transduct. Target Ther., |
| | | 1: 16002 (2016) (PMID: 29263894) |
| | Leu 16 | Lee et al., J. Immunother., 41(1): 19-31 (2018) |
| | | (PMID: 29176334) |
| | CD20 scFvs | International Patent Application Publication No. |
| | | WO/2016/164731 |
| BCMA | BCMA-02 | Raje et al., N. Engl. J. Med., 380(18): 1726-1737 |
| | | (2019) (PMID: 31042825) |
| | LCAR38 | Zhao et al., J. Hematol. Oncol., 11(1): 141 (2018) |
| | | (PMID: 30572922) |
| | BCMA scFv | Smith et al., Cancer Immunol. Res., 7(7): 1047-1053 |
| | | (2019) (PMID: 31113804) |
| | Biepitopic | Xu et al., Proc. Natl. Acad. Sci. USA, 116(19): |
| | | 9543-9551 (2019) (PMID: 30988175) |
| | NVS BCMA | Cohen et al., J. Clin. Invest., 129(6): 2210-2221 |
| | | (2019) (PMID: 30896447) |
| CS-1 | CS1R | Wang et al., Clin. Cancer Res., 24(1): 106-119 |
| | | (2018) (PMID: 29061640) |
| | CS1 ScFv | Chu et al., Leukemia, 28(4): 917-27 (2014) (PMID: |
| | | 24067492) |
| | Elotuzumab based | Dimopoulos et al., N. Engl. J. Med., 379(19): |
| | | 1811-1822 (2018) (PMID: 30403938) |
| CD138 | ScFv | Sun et al., Oncotarget., 10(24): 2369-2383 (2019) |
| | | (PMID: 31040928) |
| CD44v6 | Humanized scFv | Leuci et al., Oncoimmunology, 7(5): e1423167 |
| | | (2018) (PMID: 29721373) |
| | cMAb U36 | Sandstrom et al., Int. J. Oncol., 40(5): 1525-32 |
| | | (2012) (PMID: 22307465) |
| NKG2D | scFv | Yang et al., J. Immunother. Cancer, 7(1): 171 (2019) |
| | | (PMID: 31288857) |
| | NKG2Dg scFv | Parihar et al., Cancer Immunol. Res., 7(3): 363-375 |
| | | (2019) (PMID: 30651290) |
| CD38 | Nanobody CD38 | An et al., Mol. Pharm., 15(10): 4577-4588 (2018) |
| | | (PMID: 30185037) |
| | Humanized scFv | Yoshida et al., Clin. Transl. Immunology, |
| | | 5(12): e116 (2016) (PMID: 28090317) |
| GPRC5D | Humanized scFv | Smith et al., Sci. Transl. Med., 11(485)eaau7746 |
| | | (2019) (PMID: 30918115) |
| CD79b | scFv (L-H) and (H-L) | Ormhoj et al., Clin. Cancer Res., (2019) (PMID: |
| | | 31439577) |
| CD103 | M290 | Zhang et al., Am. J. Transplant., 9(9): 2012-23 |
| | | (2009) (PMID: 19645708) |
| FAP | FAP5 | Wang et al., Cancer Immunol. Res., 2(2): 154-66 |
| | | (2014) (PMID: 24778279) |
| CD70 | Human CD70 | Park et al., Oral Oncol., 78: 145-150 (2018) (PMID: |
| | | 29496042) |
| MUC16 | 4H11 | Koneru et al., Oncoimmunol., 4(3): e994446 (2015) |
| | | (PMID: 25949921) |
| IL13Ra2 | IL13R | Kong et al., Clin. Cancer Res., 18(21): 5949-60 |
| | | (2012) (PMID: 22966020) |

Any appropriate method can be used to express an antigen receptor on a T cell having (e.g., engineered to have) a reduced level of an Axl polypeptide. For example, a nucleic acid encoding an antigen receptor can be introduced into one or more T cells. In some cases, viral transduction can be used to introduce a nucleic acid encoding an antigen receptor into a non-dividing a cell. A nucleic acid encoding an antigen receptor can be introduced in a T cell using any appropriate method. In some cases, a nucleic acid encoding an antigen receptor can be introduced into a T cell by transduction (e.g., viral transduction using a retroviral vector such as a lentiviral vector) or transfection. In some cases, a nucleic acid encoding an antigen receptor can be introduced ex vivo into one or more T cells. For example, ex vivo engineering of T cells expressing an antigen receptor can include transducing isolated T cells with a lentiviral vector encoding an antigen receptor. In cases where T cells are engineered ex vivo to express an antigen receptor, the T cells can be obtained from any appropriate source (e.g., a mammal such as the mammal to be treated or a donor mammal, or a cell line).

In some cases, when a T cell having (e.g., engineered to have) a reduced level of an Axl polypeptide also expresses (e.g., is engineered to express) an antigen receptor, that T cell can be engineered to have a reduced level of an Axl polypeptide and engineered to express an antigen receptor using any appropriate method. In some cases, a T cell can be engineered to have a reduced level of an Axl polypeptide first and engineered to express an antigen receptor second, or vice versa. In some cases, a T cell can be simultaneously engineered to have a reduced level of an Axl polypeptide and to express an antigen receptor. For example, one or more nucleic acids used to reduce a level of an Axl polypeptide (e.g., a lentiviral vector encoding a nucleic acid molecule designed to induce RNA interference and/or a lentiviral vector encoding gene-editing components) and one or more nucleic acids encoding an antigen receptor (e.g., a CAR) can be simultaneously introduced into one or more T cells. One or more nucleic acids used to reduce a level of an Axl polypeptide and one or more nucleic acids encoding an antigen receptor can be introduced into one or more T cells on separate nucleic acid constructs or on a single nucleic acid construct. In some cases, one or more nucleic acids used to reduce a level of an Axl polypeptide and one or more nucleic acids encoding an antigen receptor can be introduced into one or more T cells on a single nucleic acid construct. In some cases, one or more nucleic acids used to reduce a level of an Axl polypeptide and one or more nucleic acids encoding an antigen receptor can be introduced ex vivo into one or more T cells. In cases where T cells are engineered ex vivo to have a reduced level of an Axl polypeptide and to express an antigen receptor, the T cells can be obtained from any appropriate source (e.g., a mammal such as the mammal to be treated or a donor mammal, or a cell line).

In some cases, a T cell having (e.g., engineered to have) a reduced level of an Axl polypeptide can be stimulated. A T cell can be stimulated at the same time as being engineered to have a reduced level of an Axl polypeptide or independently of being engineered to have a reduced level of an Axl polypeptide. For example, one or more T cells having a reduced level of an Axl polypeptide used in an adoptive cell therapy can be stimulated first, and can be engineered to have a reduced level of an Axl polypeptide second, or vice versa. In some cases, one or more T cells having a reduced level of an Axl polypeptide used in an adoptive cell therapy can be stimulated first, and can be engineered to have a reduced level of an Axl polypeptide second. A T cell can be stimulated using any appropriate method. For example, a T cell can be stimulated by contacting the T cell with one or more CD polypeptides. Examples of CD polypeptides that can be used to stimulate a T cell include, without limitation, CD3, CD28, inducible T cell co-stimulator (ICOS), CD137, CD2, OX40, CD27, MYD88, and CD40L.

This document also provides methods and materials involved in treating cancer. For example, one or more T cells having (e.g., engineered to have) a reduced level of an Axl polypeptide (e.g., Axl KO CAR T cells) can be administered (e.g., in an adoptive cell therapy such as a CAR T cell therapy) to a mammal (e.g., a human) having cancer to treat the mammal. In some cases, methods of treating a mammal having cancer as described herein can reduce the number of cancer cells (e.g., cancer cells expressing a tumor antigen) within a mammal. In some cases, methods of treating a mammal having cancer as described herein can reduce the size of one or more tumors (e.g., tumors expressing a tumor antigen) within a mammal.

Any appropriate amount (e.g., number) of T cells having (e.g., engineered to have) a reduced level of an Axl polypeptide (e.g., Axl KO CAR T cells) can be administered (e.g., in an adoptive cell therapy such as a CAR T cell therapy) to a mammal (e.g., a human) having cancer. In some cases, from about 0.1×10⁶ T cells (e.g., CAR T cells) to about 10×10⁶ T cells (e.g., CAR T cells) having a reduced level of an Axl polypeptide (e.g., Axl KO CAR T cells) per kilogram (kg) body weight of the mammal (e.g., from about 0.1×10⁶ to about 9×10⁶, from about 0.1×10⁶ to about 8×10⁶, from about 0.1×10⁶ to about 7×10⁶, from about 0.1×10⁶ to about 6×10⁶, from about 0.1×10⁶ to about 5×10⁶, from about 0.1×10⁶ to about 4×10⁶, from about 0.1×10⁶ to about 3×10⁶, from about 0.1×10⁶ to about 2×10⁶, from about 0.1×10⁶ to about 1×10⁶, from about 0.1×10⁶ to about 0.5×10⁶, from about 0.5×10⁶ to about 10×10⁶, from about 1×10⁶ to about 10×10⁶, from about 2×10⁶ to about 10×10⁶, from about 3×10⁶ to about 10×10⁶, from about 4×10⁶ to about 10×10⁶, from about 5×10⁶ to about 10×10⁶, from about 6×10⁶ to about 10×10⁶, from about 7×10⁶ to about 10×10⁶, from about 8×10⁶ to about 10×10⁶, from about 9×10⁶ to about 10×10⁶, from about 0.5×10⁶ to about 9×10⁶, from about 1×10⁶ to about 8×10⁶, from about 2×10⁶ to about 7×10⁶, from about 3×10⁶ to about 6×10⁶, from about 4×10⁶ to about 5×10⁶, from about 1×10⁶ to about 3×10⁶, from about 2×10⁶ to about 4×10⁶, from about 3×10⁶ to about 5×10⁶, from about 4×10⁶ to about 6×10⁶, from about 5×10⁶ to about 7×10⁶, from about 6×10⁶ to about 8×10⁶, or from about 7×10⁶ to about 9×10⁶ of T cells per kg) can be administered to a mammal having cancer to treat the mammal.

Any appropriate mammal (e.g., a human) having a cancer can be treated as described herein. Examples of mammals that can be treated as described herein include, without limitation, humans, non-human primates (e.g., monkeys), dogs, cats, horses, cows, pigs, sheep, mice, and rats. For example, a human having a cancer can be treated with one or more T cells having (e.g., engineered to have) a reduced level of an Axl polypeptide (e.g., Axl KO CAR T cells) in, for example, an adoptive T cell therapy such as a CAR T cell therapy using the methods and materials described herein.

When treating a mammal (e.g., a human) having a cancer as described herein, the cancer can be any appropriate cancer. In some cases, a cancer treated as described herein can include one or more solid tumors. In some cases, a cancer treated as described herein can be a hematological (blood) cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. In some cases, a cancer treated as described herein can express a tumor-associated antigen (e.g., an antigenic substance produced by a cancer cell). Examples of cancers that can be treated as described herein include, without limitation, diffuse large B cell lymphoma (DLBCL), Hodgkin's lymphomas, non-Hodgkin lymphomas, acute lymphoblastic leukemia (ALL), chronic lymphocytic leukemia (CLL), acute myeloid leukemia (AML), germ cell tumors, hepatocellular carcinoma, bowel cancer, lung cancer, breast cancer, ovarian cancer, melanoma, epithelial tumors, brain cancers, multiple myelomas, sarcomas, bone cancers, pancreatic cancers, liver cancers, myeloid neoplasms, prostate cancers, and thyroid cancers.

In some cases, the methods described herein can include identifying a mammal (e.g., a human) as having a cancer. Any appropriate method can be used to identify a mammal having cancer. For example, imaging techniques and biopsy techniques can be used to identify mammals (e.g., humans) having cancer.

In some cases, the methods described herein can include identifying a mammal (e.g., a human) as being in need of T cells (e.g., CAR T cells) having enhanced anti-tumor activity. Any appropriate method can be used to identify a mammal as being in need of T cells (e.g., CAR T cells) having enhanced anti-tumor activity. For example, medical histories (e.g., evaluations of disease state and/or knowledge of response to prior therapies) and/or diagnosis (e.g., diagnosis with a cancer that is difficult to treat such as hematological cancer that is difficult to treat) can be used to identify mammals (e.g., humans) as being in need of such T cells (e.g., such CAR T cells).

A mammal (e.g., a human) having a cancer can be administered one or more T cells having (e.g., engineered to have) a reduced level of an Axl polypeptide (e.g., Axl KO CAR T cells) described herein. For example, one or more T cells having (e.g., engineered to have) a reduced level of an Axl polypeptide can be used in an adoptive T cell therapy (e.g., a CAR T cell therapy) to treat a mammal having a cancer. For example, one or more T cells having a reduced level of an Axl polypeptide can be used in an adoptive T cell therapy (e.g., a CAR T cell therapy) targeting any appropriate antigen within a mammal (e.g., a mammal having cancer). In some cases, an antigen can be a tumor-associated antigen (e.g., an antigenic substance produced by a cancer cell). Examples of tumor-associated antigens that can be targeted by an adoptive T cell therapy provided herein include, without limitation, CD19 (associated with DLBCL, ALL, and CLL), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), MAGE (associated with malignant melanoma), CD33 (associated with AML), CD123 (associated with AML), CLL-1 (associated with AML), E-Cadherin (associated with epithelial tumors), folate receptor alpha (associated with ovarian cancers), folate receptor feta (associated with ovarian cancers and AML), IL13R (associated with brain cancers), EGFRviii (associated with brain cancers), CD22 (associated with B cell cancers), CD20 (associated with B cell cancers), kappa light chain (associated with B cell cancers), lambda light chain (associated with B cell cancers), CD44v (associated with AML), CD45 (associated with hematological cancers), CD30 (associated with Hodgkin lymphomas and T cell lymphomas), CD5 (associated with T cell lymphomas), CD7 (associated with T cell lymphomas), CD2 (associated with T cell lymphomas), CD38 (associated with multiple myelomas and AML), BCMA (associated with multiple myelomas), CD138 (associated with multiple myelomas and AML), FAP (associated with solid tumors), CS-1 (associated with multiple myeloma), TSHR (associated with thyroid cancers), and c-Met (associated with breast cancer). For example, one or more T cells having a reduced level of an Axl polypeptide can be used in CAR T cell therapy targeting CD19 (e.g., a CART19 cell therapy) to treat cancer as described herein.

In some cases, one or more T cells having (e.g., engineered to have) a reduced level of an Axl polypeptide (e.g., Axl KO CAR T cells) used in an adoptive T cell therapy (e.g., a CAR T cell therapy) can be administered to a mammal having a cancer as a combination therapy with one or more additional agents used to treat a cancer. For example, one or more T cells having a reduced level of an Axl polypeptide used in an adoptive cell therapy can be administered to a mammal in combination with one or more anti-cancer treatments (e.g., surgery, radiation therapy, chemotherapy (e.g., alkylating agents such as busulfan). In cases where one or more T cells having a reduced level of an Axl polypeptide used in an adoptive cell therapy are used with additional agents treat a cancer, the one or more additional agents can be administered at the same time or independently. In some cases, one or more T cells having a reduced level of an Axl polypeptide used in an adoptive cell therapy can be administered first, and the one or more additional agents administered second, or vice versa.

In some cases, the methods and materials described herein can be applied to immune cells other than T cells, such as natural killer (NK) cells. For example, the methods and materials described herein can be used to design NK cells having (e.g., engineered to have) a reduced level of an Axl polypeptide (e.g., Axl KO CAR-NK cells). In some cases, NK cells having a reduced level of an Axl polypeptide can be used in an adoptive cell therapy (e.g., a CAR-NK cell therapy). For example, NK cells having a reduced level of an Axl polypeptide can be administered to a mammal having a cancer to treat the cancer. For example, NK cells having a reduced level of an Axl polypeptide can be administered to a mammal having a cancer as a combination therapy with one or more additional agents used to treat a cancer.

### Exemplary Embodiments

Embodiment 1 is a method of generating a T cell having a reduced level of an Axl polypeptide, wherein said method comprises:
(a) introducing, into a T cell, a nucleic acid encoding a Cas nuclease and a guide RNA, and
(b) culturing said T cell under conditions in which said Cas nuclease and said guide RNA are expressed and said Cas nuclease cleaves genomic DNA within said T cell at a nucleotide sequence targeted by said guide RNA, wherein said nucleic acid encoding said guide RNA comprises the sequence set forth in SEQ ID NO:1 or the sequence set forth in any one of the sequence identifiers set forth in TABLE 1.

Embodiment 2 is the method of embodiment 1, wherein said T cell having a reduced level of an Axl polypeptide comprises a disruption in at least one endogenous allele encoding said Axl polypeptide.

Embodiment 3 is the method of embodiment 1, wherein said T cell having a reduced level of an Axl polypeptide comprises a disruption in both endogenous alleles encoding said Axl polypeptide.

Embodiment 4 is the method of embodiment 2 or embodiment 3, wherein said T cell expresses a reduced level of said Axl polypeptide as compared to a comparable T cell lacking said disruption.

Embodiment 5 is the method of any one of embodiments 1 to 4, wherein said method further comprises introducing, into said T cell, a nucleic acid encoding a chimeric antigen receptor.

Embodiment 6 is the method of embodiment 5, wherein said introducing of said nucleic acid encoding said chimeric antigen receptor is performed before said introducing of said nucleic acid encoding said Cas nuclease and said guide RNA.

Embodiment 7 is the method of embodiment 5, wherein said introducing of said nucleic acid encoding said chimeric antigen receptor is performed after said introducing of said nucleic acid encoding said Cas nuclease and said guide RNA.

Embodiment 8 is the method of embodiment 5, wherein said chimeric antigen receptor targets a tumor-associated antigen.

Embodiment 9 is the method of embodiment 8, wherein said tumor-associated antigen is CD19.

Embodiment 10 is the method of any one of embodiments 1 to 9, wherein said T cell is obtained from a human.

Embodiment 11 is a T cell comprising (a) a reduced level of an Axl polypeptide, and (b) nucleic acid encoding a chimeric antigen receptor, wherein said T cell expresses said chimeric antigen receptor, and wherein said T cell was generated by a method that comprises introducing, into said T cell or a precursor thereof, a Cas nuclease and a nucleotide sequence encoding a guide RNA, said nucleotide sequence encoding said guide RNA comprises the sequence set forth in SEQ ID NO:1 or the sequence set forth in any one of the sequence identifiers set forth in TABLE 1, such that said Cas nuclease cleaves genomic DNA within said T cell at a nucleotide sequence targeted by said guide RNA.

Embodiment 12 is the T cell of embodiment 11, wherein said T cell comprises a disruption in at least one endogenous allele encoding said Axl polypeptide.

Embodiment 13 is the T cell of embodiment 11, wherein said T cell comprises a disruption in both endogenous alleles encoding said Axl polypeptide.

Embodiment 14 is the T cell of embodiment 12 or embodiment 13, wherein said T cell expresses a reduced level of said Axl polypeptide as compared to a comparable T cell lacking said disruption.

Embodiment 15 is the T cell of any one of embodiments 11 to 14, wherein said chimeric antigen receptor targets a tumor-associated antigen.

Embodiment 16 is the T cell of embodiment 15, wherein said tumor-associated antigen is CD19.

Embodiment 17 is the T cell of any one of embodiments 11 to 16, wherein said T cell is obtained from a human.

Embodiment 18 is a method for treating a mammal having cancer, wherein said method comprises administering to said mammal a composition comprising the T cell of any one of embodiments 11 to 17.

Embodiment 19 is the method of embodiment 18, wherein said mammal is a human.

Embodiment 20 is the method of embodiment 18 or embodiment 19, wherein said composition comprises from about 0.1×10⁶ to about 10×10⁶ of said T cells.

Embodiment 21 is the method of any one of embodiments 18 to 20, wherein said cancer is selected from diffuse large B cell lymphoma (DLBCL), Hodgkin's lymphomas, non-Hodgkin lymphomas, acute lymphoblastic leukemia (ALL), chronic lymphocytic leukemia (CLL), acute myeloid leukemia (AML), germ cell tumors, hepatocellular carcinoma, bowel cancer, lung cancer, breast cancer, ovarian cancer, melanoma, epithelial tumors, brain cancers, multiple myelomas, sarcomas, bone cancers, pancreatic cancers, liver cancers, myeloid neoplasms, prostate cancers, and thyroid cancers.

Embodiment 22 is the use of a composition comprising the T cell of any one of embodiments 11 to 17 to treat a mammal having cancer.

Embodiment 23 is a composition comprising the T cell of any one of embodiments 11 to 17 for use in the preparation of a medicament to treat a mammal having cancer.

Embodiment 24 is a composition comprising the T cell of any one of embodiments 11 to 17 for use in the treatment of cancer.

Embodiment 25 is a method of generating a T cell having a reduced level of an Axl polypeptide, wherein said method comprises (a) introducing, into a T cell, nucleic acid encoding a transcription activator-like effector (TALE) nuclease, wherein said TALE nuclease comprises a first monomer and a second monomer, and (b) culturing said T cell under conditions in which said first TALE nuclease monomer and said second TALE nuclease monomer are expressed within said T cell, wherein said first TALE nuclease monomer binds to a first target sequence in genomic DNA of said T cell, wherein said second TALE nuclease monomer binds to a second target sequence in said genomic DNA, and wherein said first and second TALE nuclease monomers dimerize and cleave said genomic DNA of said T cell, wherein said first and second target sequences comprise a pair of nucleotide sequences set forth in the sequence identifier pairs that are set forth in TABLE 2.

Embodiment 26 is the method of embodiment 25, wherein said T cell having a reduced level of an Axl polypeptide comprises a disruption in at least one endogenous allele encoding said Axl polypeptide.

Embodiment 27 is the method of embodiment 25, wherein said T cell having a reduced level of an Axl polypeptide comprises a disruption in both endogenous alleles encoding said Axl polypeptide.

Embodiment 28 is the method of embodiment 26 or embodiment 27, wherein said T cell expresses a reduced level of said Axl polypeptide as compared to a comparable T cell lacking said disruption.

Embodiment 29 is the method of any one of embodiments 25 to 28, wherein said method further comprises introducing, into said T cell, a nucleic acid encoding a chimeric antigen receptor.

Embodiment 30 is the method of embodiment 29, wherein said introducing of said nucleic acid encoding said chimeric antigen receptor is performed before said introducing of said nucleic acid encoding said TALE nuclease.

Embodiment 31 is the method of embodiment 29, wherein said introducing of said nucleic acid encoding said chimeric antigen receptor is performed after said introducing of said nucleic acid encoding said TALE nuclease.

Embodiment 32 is the method of any one of embodiments 29 to 31, wherein said chimeric antigen receptor targets a tumor-associated antigen.

Embodiment 33 is the method of embodiment 32, wherein said tumor-associated antigen is CD19.

Embodiment 34 is the method of any one of embodiments 25 to 33 wherein said T cell is obtained from a human.

Embodiment 35 is a T cell comprising (a) a reduced level of an Axl polypeptide, and (b) nucleic acid encoding a chimeric antigen receptor, wherein said T cell expresses said chimeric antigen receptor, and wherein said T cell was generated by a method that comprises: (i) introducing, into said T cell or a precursor thereof, a transcription activator-like effector (TALE) nuclease, wherein said TALE nuclease comprises a first monomer and a second monomer, and (ii) culturing said T cell under conditions in which said first TALE nuclease monomer and said second TALE nuclease monomer are expressed within said T cell, wherein said first TALE nuclease monomer binds to a first target sequence in genomic DNA of said T cell, wherein said second TALE nuclease monomer binds to a second target sequence in said genomic DNA, and wherein said first and second TALE nuclease monomers dimerize and cleave said genomic DNA of said T cell, wherein said first and second target sequences comprise a pair of nucleotide sequences set forth in the sequence identifier pairs that are set forth in TABLE 2.

Embodiment 36 is the T cell of embodiment 35, wherein said T cell comprises a disruption in at least one endogenous allele encoding said Axl polypeptide.

Embodiment 37 is the T cell of embodiment 35, wherein said T cell comprises a disruption in both endogenous alleles encoding said Axl polypeptide.

Embodiment 38 is the T cell of embodiment 36 or embodiment 37, wherein said T cell expresses a reduced level of said Axl polypeptide as compared to a comparable T cell lacking said disruption.

Embodiment 39 is the T cell of any one of embodiments 35 to 38, wherein said chimeric antigen receptor targets a tumor-associated antigen.

Embodiment 40 is the T cell of embodiment 39, wherein said tumor-associated antigen is CD19.

Embodiment 41 is the T cell of any one of embodiments 35 to 40, wherein said T cell is obtained from a human.

Embodiment 42 is a method for treating a mammal having cancer, wherein said method comprises administering to said mammal a composition comprising the T cell of any one of embodiments 35 to 41.

Embodiment 43 is the method of embodiment 42, wherein said mammal is a human.

Embodiment 44 is the method of embodiment 42 or embodiment 43, wherein said composition comprises from about 0.1×10⁶ to about 10×10⁶ of said T cells.

Embodiment 45 is the method of any one of embodiments 42 to 44, wherein said cancer is selected from diffuse large B cell lymphoma (DLBCL), Hodgkin's lymphomas, non-Hodgkin lymphomas, acute lymphoblastic leukemia (ALL), chronic lymphocytic leukemia (CLL), acute myeloid leukemia (AML), germ cell tumors, hepatocellular carcinoma, bowel cancer, lung cancer, breast cancer, ovarian cancer, melanoma, epithelial tumors, brain cancers, multiple myelomas, sarcomas, bone cancers, pancreatic cancers, liver cancers, myeloid neoplasms, prostate cancers, and thyroid cancers.

Embodiment 46 is the use of a composition comprising the T cell of any one of embodiments 35 to 41 to treat a mammal having cancer.

Embodiment 47 is a composition comprising the T cell of any one of embodiments 35 to 41 for use in the preparation of a medicament to treat a mammal having cancer.

Embodiment 48 is a composition comprising the T cell of any one of embodiments 35 to 41 for use in the treatment of cancer.

The invention will be further described in the following examples, which do not limit the scope of the invention described in the claims.

### EXAMPLES

### Example 1-Axl Inhibition Improves CAR T Cell Therapy Through Selective Targeting of Th2 Cell and M2-Type Macrophages

### Methods and Materials

Cell lines and primary samples: JeKo-1 and Molm-13 cells were obtained from American Tissue Culture Collection (ATCC; Manassas, VA). For indicated experiments, JeKo-1 and Molm-13 cells were transduced with the firefly luciferase ZsGreen (Addgene; Cambridge, MA) and then sorted to obtain a greater than 99% positive population as described elsewhere (Sterner et al., J Vis Exp: Jove, 2019a, DOI: 10.3791/59629; and Sterner et al., Blood, 133:697-709, 2019b). Cell lines were cultured in R10 medium made with Roswell Park Memorial Institute (RPMI) 1640 (Gibco; Gaithersburg, MD), 10% fetal bovine serum (FBS) (Sigma, St. Louis, MO, USA), and 1% penicillin-streptomycin-glutamine (Gibco). All cell lines were tested for mycoplasma (IDEXX; Columbia, MO) and found to be negative.

Peripheral blood specimens were obtained from solid tumor patients. Peripheral blood samples from de-identified normal donor blood apheresis cones (Dietz et al., Transfusion, 46:2083-2089, 2006) were obtained and PBMCs were isolated using SepMate tubes (STEMCELL™ Technologies). T cells were separated with negative selection magnetic beads using a EASYSEP™ Human T Cell Isolation Kit (STEMCELL™ Technologies). CLL peripheral blood specimens were obtained from the prospectively maintained Mayo Clinic CLL biobank.

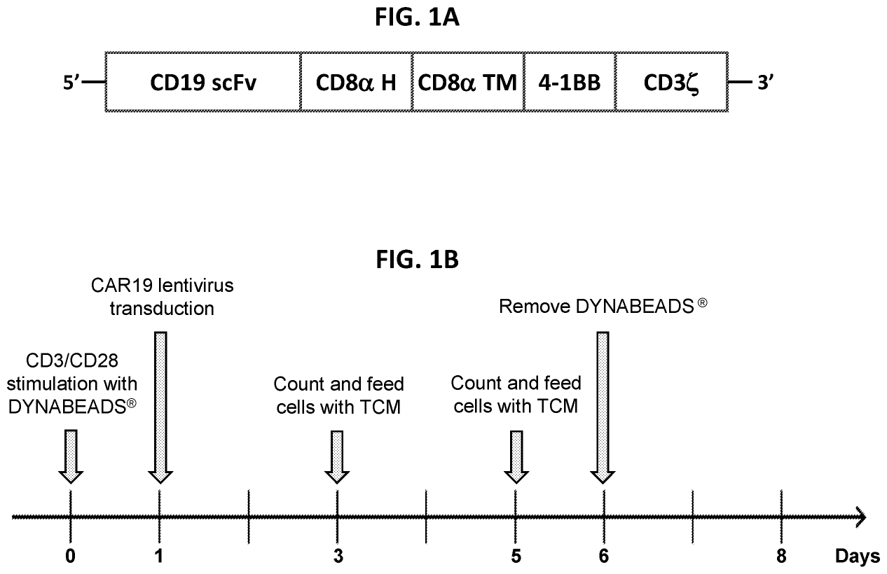

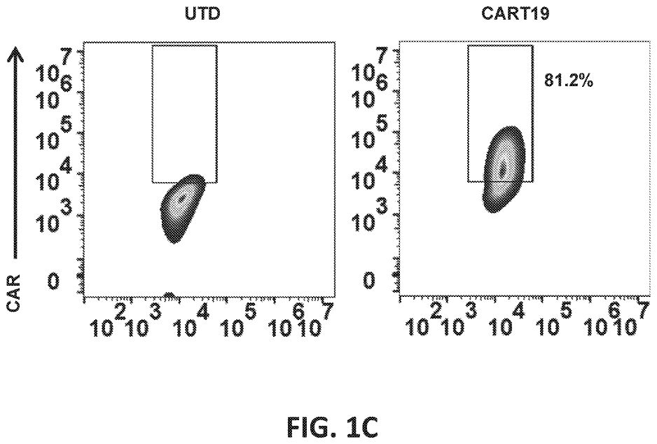

Generation of CAR constructs and CAR T cells: A murine CAR19 plasmid (pLV-CAR19) was generated by cloning anti-CD19 scFv, CD8 hinge and transmembrane domain, 4-1BB costimulatory domain and CD32 signaling domain into a lentiviral backbone (FIG. 1A) as described elsewhere (Sterner et al. 2019a, supra; and Sterner et al. 2019b, supra). T cells from normal healthy donors were expanded in vitro with anti-CD3/CD28 Dynabeads (Invitrogen/Life Technologies; Grand Island, NY) added on day 0 of culture at a bead: cell ratio of 3:1. T cells were transduced with lentiviral supernatant from 293T cells transfected with the pLV-CAR19 plasmid and two helper plasmids on day 1 at a multiplicity of infection of three (FIG. 1B). The anti-CD3/CD28 Dynabeads were removed on day 6, and flow cytometric analysis for CAR expression was performed with goat anti-mouse IgG (H+L) cross-adsorbed secondary antibody (Invitrogen/Life Technologies) (FIG. 1C).

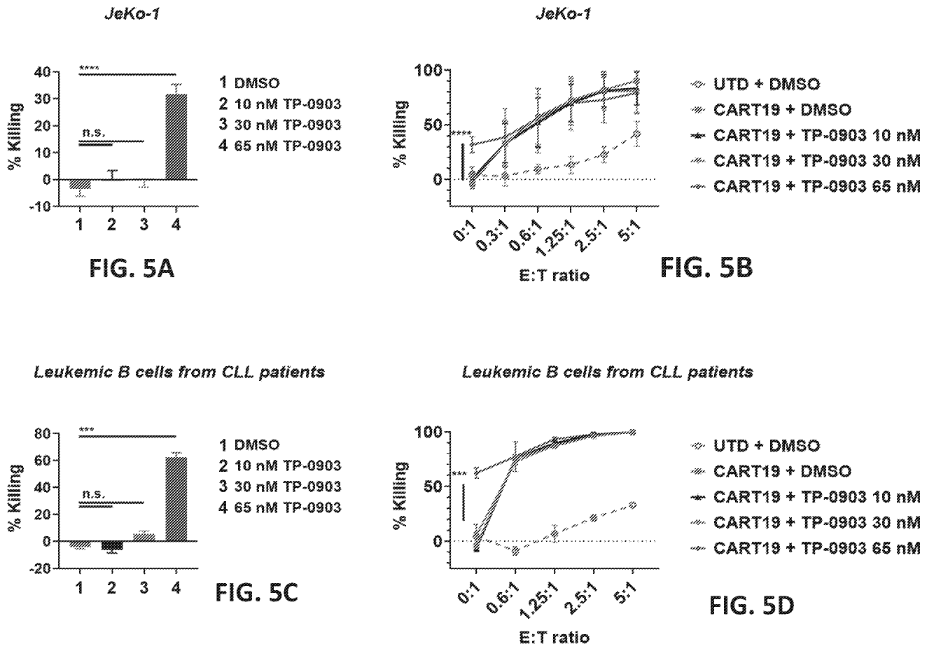

Untransduced T cells (UTD) or CART19 cells were grown in T cell media (TCM) [X-VIVO™ 15 media (Lonza; Basel, Switzerland), 10% human AB serum (Corning, NY), 1% penicillin-streptomycin-glutamine (Gibco)] for up to 8 days and then cryopreserved for future experiments. Prior to all experiments, T cells were thawed and rested overnight at 37° C., 5% CO₂ (FIG. 5B).

Generation of Axl^{ko} CART19 cells: A nucleotide sequence encoding a gRNA targeted to the second exon of human AXL (5′-TTCGGTGTCAGCTCCAGGTT-3′; SEQ ID NO: 1) (see, Axelrod, supra) was cloned into pLenti CRISPRv2 (GenScript; Township, NJ), a lentiviral vector carrying Cas9 and gRNA under the control of a U6 promoter (Sanjana et al., Nat Methods, 11:783-784, 2014). Axl^{ko} CART19 cells were then manufactured via dual transduction of CAR19 and CRISPR lentiviruses. Disruption efficiency of CRISPR/Cas9 Axl knockout was determined using targeted sequencing through PCR (Polymerase Chain Reaction) and TIDE (Tracking of Indels by Decomposition) analysis, the latter using software available at tide.nki.nl as described elsewhere (Cox et al., supra; and Brinkman et al., Nucl Acids Res, 42 (22): e168, 2014). Control, Axl^{wt} CART19 cells were generated using a non-targeting scrambled guide RNA as reported elsewhere (Cox et al., supra).

TP-0903: TP-0903 was obtained from Tolero Pharmaceuticals, Inc. (Lehi, Utah). For in vitro experiments, TP-0903 was dissolved in DMSO and diluted to 10, 30, or 65 nM in culture media. For in vivo experiments, TP-0903 powder was dissolved in 5% (w/v) vitamin E TPGS (Sigma, St. Louis, MO, USA)+1% (v/v) TWEEN™ 80 (Sigma; St. Louis, MO) in deionized water.

Monocyte/macrophage differentiation: Fresh blood samples from healthy donors were collected, and PBMCs were isolated by density gradient centrifugation using SepMate-50 tube (STEMCELL™ Technologies). Isolation of monocytes was performed using a classical monocyte isolation kit (Miltenyi Biotec; Bergisch Gladbach, Germany) according to the manufacturer's protocol. After isolation of CD14⁺ monocytes, cells were cultured in IMMUNOCULT™-SF Macrophage Medium (STEMCELL™ Technologies) along with 5 μg/mL recombinant human recombinant macrophage colony stimulating factor (M-CSF). Monocytes were then cultured at 37° C. in a humidified incubator with 5% CO₂ until day 4. For M1 differentiation, 10 ng/mL LPS and 50 ng/ml IFN-γ (STEMCELL™ Technologies) were added to the culture. For M2 differentiation, 10 ng/mL IL-4 (STEMCELL™ Technologies) was added to the culture according to the manufacturer's protocol. Macrophages were harvested on day 6 and used in the assays.

Treg cell isolation: CD4⁺ T cells derived from healthy donors were enriched by negative selection and subsequently segregated into a CD4⁺ CD25⁺ CD127_{low} subpopulation by magnetic bead separation using the EASYSEP™ Human T Cell Isolation Kit (STEMCELL™ Technologies). Following this, CD4⁺ CD25⁺ CD127_{low} cells were sorted on a FACSAria III sorter (BD Biosciences Pharmingen; Heidelberg, Germany) to obtain CD4⁺ CD25_{high} CD127_{low}CD45RA⁺ Tregs (purity of >90%).

Treg suppression assay: Responder T cells (Tresp) were isolated with the similar technique using as described in the Cell lines and primary samples section above, using SepMate tubes and a EASYSEP™ Human T Cell Isolation Kit. Tresp were stained with carboxyfluorescein succinimidyl ester (CFSE), and the Treg and CFSE stained Tresp were co-cultured at the indicated Treg: Tresp ratios in the presence or absence of 30 nM of TP-0903 for 4 days. At the end of the culture period, cells were stained and flow cytometric analysis was performed. The percent suppression of Tresp was calculated based on the percent dividing cells.

Multi-parametric flow cytometry: Anti-human antibodies were purchased from Biolegend (San Diego, CA), eBioscience (San Diego, CA), or BD Biosciences (San Jose, CA); see, TABLE 4. The preparation of samples for flow cytometry was a described elsewhere (Sterner et al. 2019a, supra; and Sterner et al. 2019b, supra). For cell number quantitation, COUNTBRIGHT™ beads (Invitrogen; Carlsbad, CA) were used according to the manufacturer's instructions. In all analyses, the population of interest was gated based on forward vs side scatter characteristics, followed by singlet gating, followed by live cells gating with LIVE/DEAD™ Fixable Aqua Dead Cell Stain Kit (Invitrogen). One hundred thousand (1×10⁵) live cells were collected in each assay. Flow cytometry was performed on a three-laser CytoFLEX (Beckman Coulter; Chaska, MN). All analyses were performed using FlowJo X10.0.7r2 software (Ashland, OR).

TABLE 4
Antibody Sources

| Name | Fluorochrome | Clone | Vendor | Catalog |
|---|---|---|---|---|
| Goat anti-mouse | AF647 | | Invitrogen | A21235 |
| IgG (H + L) cross- | | | | |
| adsorbed secondary | | | | |
| antibody | | | | |
| CD14 | APC | 61D3 | eBioscience | 17-0149-41 |
| CD14 | FITC | 63D3 | BioLegend | 367115 |
| CD19 | PE | HIB19 | eBioscience | 12-0199-42 |
| CD19 | PE-Cy7 | SJ25C1 | eBioscience | 25-0198-42 |
| CD19 | APC | HIB19 | BioLegend | 302212 |
| CD20 | FITC | L27 | BD Biosciences | 347673 |
| CD20 | PE-Cy7 | 2H7 | eBioscience | 25-0209-42 |
| CD20 | APC | 2H7 | BioLegend | 302310 |
| CD4 | BV421 | RPA-T4 | BD Horizon | 562424 |
| CD4 | FITC | OKT4 | eBioscience | 11-0048-42 |
| CD4 | PE-Cy7 | SK3 | BD Biosciences | 557852 |
| CD3 | PE-Cy7 | OKT3 | BioLegend | 317334 |
| CD3 | PE | OKT3 | eBioscience | 12-0037-42 |
| CD3 | APC-Cy7 | SK7 | BioLegend | 344818 |
| CD3 | BV605 | SK7 | BioLegend | 344836 |
| CD3 | APC | UCHT1 | Invitrogen | 17-0038-42 |
| CD8 | PerCP | SK1 | BioLegend | 344707/344708 |
| CD25 | PE | BC96 | BioLegend | 302606 |
| CD127 | FITC | HIL-7R-M21 | BD Biosciences | 561697 |
| CD45RA | APC | HI100 | BioLegend | 304111/304112 |
| CD107a | FITC | H4A3 | BioLegend | 328606 |
| IL-2 | PE-CF594 | 5344.111 | BD Pharmingen | 562384 |
| GM-CSF | BV421 | BVD2-21C11 | BD Horizon | 562930 |
| IFN-γ | AF700 | Mab11 | BioLegend | 502928 |
| TNF-α | PE-Cy7 | D21-1351 | BD Pharmingen | 560687 |
| IL-4 | APC | MP4-25D2 | BD Pharmingen | 554486 |
| IL-13 | PE-Cy7 | JES10-5A2 | BioLegend | 501914 |
| Axl | unconjugated | polyclonal | R&D systems | AF154 |
| Goat IgG (H + L) | APC | polyclonal | R&D systems | F0108 |
| APC-conjugated | | | | |
| antibody | | | | |
| Anti mouse CD45 | APC-eFluor780 | 30-F11 | Invitrogen | 47-0451-82 |
| Anti human CD45 | BV421 | HI30 | BioLegend | 304032 |

In vitro T cell proliferation assays: Naïve T cells or CART19 cells were labeled with carboxyfluorescein succinimidyl ester (CFSE) (Invitrogen) as described elsewhere (Sterner et al. 2019a, supra; and Sterner et al. 2019b, supra). Cells were then re-suspended in TCM at 2×10⁶/mL, and 50 μL per well were seeded in 96-well plates. TP-0903 was added to the corresponding wells at a final concentration of 10, 30, or 65 nM. Each assay also included cells with media as a blank control, cells with PMA & ionomycin as a positive control, and cells with DMSO as negative control. After 120 hours, cells were harvested and stained for APC-H7 anti-human CD3 (eBioscience), BV421 anti-human CD4 (BioLegend), and LIVE/DEAD™ Fixable Aqua. COUNTBRIGHT™ beads were added prior to flow cytometric analysis to determine the absolute counts.

In vitro T cell cytotoxicity assays: Cytotoxicity assays were performed as described elsewhere (Sterner et al. 2019a, supra; and Sterner et al. 2019b, supra). In brief, luciferase⁺ JeKo-1 (CD19⁺) or luciferase+Molm-13 (CD19⁻) cells were used as target cells. CART19 cells were co-cultured with target cells at the indicated effector: target (E:T) ratios in TCM. Different concentrations of TP-0903 (10, 30, or 65 nmol/mL) or DMSO were added to the CART19 cells. Each assay also included control UTDs generated from the same donor and expanded under the same conditions, and a negative control target cell line. Killing was calculated by bioluminescence imaging on a Xenogen IVIS-200 Spectrum (PerkinElmer; Hopkinton, MA) at 24, 48, and 72 hours.

In vitro T cell degranulation and intracellular cytokine assays: Degranulation assays were performed as described elsewhere (Sterner et al. 2019a, supra; and Sterner et al. 2019b, supra). Briefly, T cells treated with TP-0903 were incubated with target cells at an effector: target ratio of 1:5. FITC anti-human CD107a (BD Pharmingen), anti-human CD28 (BD Biosciences), anti-human CD49d (BD Biosciences) and monensin (Biolegend) were added prior to the incubation. After 4 hours, cells were harvested and stained with LIVE/DEAD™ Fixable Aqua. Cells were then fixed and permeabilized (FIX & PERM™ Cell Fixation & Cell Permeabilization Kit) (Life Technologies; Oslo, Norway) and stained for APC anti-human CD3 (clone UCHT1; eBioscience) or BV605 anti-human CD3 (clone SK3) (Biolegend) and intracellular cytokines including PE-CF594 anti-human IL-2 (clone 5344.111; BD Pharmingen), BV421 anti-human GM-CSF (clone BVD2-21C11; BD Pharmingen), APC-eFluor 780 anti-human IFN-γ (clone 4S.B3; Invitrogen), and PE-Cy7 anti-human IL-13 (clone JES10-5A2; Biolegend), APC anti-human IL-4 (clone MP4-25D2; BD Pharmingen), and Alexa fluor 700 anti-human TNF-α (clone D21-1351; BD Pharmingen).

In vitro T cell Axl surface staining: To determine Axl expression on T cells, JeKo-1, monocytes, and macrophages, cells were stained with goat anti-human Axl affinity-purified polyclonal antibody (Catalog #AF154, R&D Systems; Minneapolis, MN) followed by APC-conjugated anti-goat IgG secondary antibody (Catalog #F0108, R&D Systems).

Cytokine analysis: Cytokine analysis was performed on cell supernatant obtained from the proliferation assays at 72 hours. Debris was removed from the supernatant by centrifugation at 10,000×g for 5 minutes. The supernatant was then diluted 1:2 with assay buffer before following the manufacturer's protocol for Milliplex Human Cytokine/Chemokine MAGNETIC BEAD Premixed 38 Plex Kit (HCYTMAG-60K-PX38) (Millipore Sigma; Ontario, Canada). Data were collected using Luminex (Millipore Sigma).

Western blot analysis: Samples were centrifuged at 100,000×g for 3 hours at 4° C., washed with PBS and centrifuged again under the same conditions. Pellets were resuspended in 100 μL of radioimmunoprecipitation assay (RIPA) buffer and protein concentration was measured using a bicinchoninic acid (BCA) protein assay (Thermo Fisher; Waltham, MA). Thirty μg of protein lysates were used for SDS-PAGE electrophoresis. Following transfer, nitrocellulose membranes were blocked with 5% bovine serum albumin in tris buffered saline with TWEEN™ (TBST) for 1 hour at room temperature. Membranes were incubated overnight at 4° C. with 1:1000 dilutions of the following antibodies: rabbit pSAPK/JNK (Thr183/Tyr185; Cell Signaling Technology; Danvers, MA), rabbit JNK (Cell Signaling Technology), rabbit pMAPK (Thr180/Tyr182; Cell Signaling Technology), rabbit MAPK (Cell Signaling Technology), rabbit pLCK (Y34; Abcam; Cambridge, MA), LCK (Abcam), rabbit GATA-3 (BD Biosciences), and rabbit T-bet (eBioscience). Membranes were washed with TBST and incubated with horseradish peroxidase-(HRP-) conjugated secondary antibodies at a dilution of 1:10,000 for 1 hour at room temperature, followed by revelation using the SuperSignal West Pico Plus Chemiluminescence substrate (Thermo Fisher).

In vivo mouse experiments: 6- to 8-week-old non-obese diabetic/severe combined immunodeficient (NOD-SCID) mice bearing a targeted mutation in the interleukin (IL)-2 receptor gamma chain gene (NSG) mice were obtained from Jackson Laboratories (Bar Harbor, ME). Mice were maintained in an animal barrier space that approved for BSL2+ level experiments. Mice were intravenously injected with 1.0×10⁶ luciferase⁺ JeKo-1 cells. 7 or 14 days after injection, mice were imaged with a bioluminescent imager using a Xenogen IVIS-200 Spectrum camera (PerkinElmer) to confirm engraftment. Imaging was performed 10 minutes after the intraperitoneal injection of 10 μL/g D-luciferin (15 mg/mL) (Gold Biotechnology; St. Louis, MO). Mice were then randomized based on their bioluminescence imaging to receive different treatments as outlined for each specific experiment. Mice were euthanized for necropsy on day 49 or when moribund or upon the development of hind-limb paralysis. To assess CAR T cell expansion in vivo, mice were bled 17 days after CART19 cell administration. 100 μL blood samples were harvested by tail vein bleeding into Microvette capillary blood tubes (Sarstedt INC MS; Nümbrecht, Germany). 70 μL of blood was lysed with RBC lysing solution (BD Biosciences; San Diego, CA), and blood cells were stained with APC-eFluor780 anti-mouse CD45 (clone 30-F11) (Invitrogen, Life Technologies; Grand Island, NY), BV421 anti-human CD45 (clone HI30) (Biolegend), PE-Cy7 anti-human CD3 (clone OKT3) (Biolegend), and APC anti-human CD20 (clone 2H7) (Biolegend). Circulating T cells were gated via mouse CD45″ human CD45⁺ CD3⁺ CD20 population. The absolute number of T cells was calculated using volume metrics (Cox et al., Mol Ther, 29:1529-1540, 2021).

RNA sequencing and analysis: CART19 cells from three biological replicates were thawed and stimulated with intact Jeko-1 cells at a one-to-one ratio for 24 hours. Each sample was treated with either 30 nM TP-0903 (treated condition) or DMSO (untreated control). CART19 cells were isolated using CD4 and CD8 microbeads to extract the JeKo-1 target cells; this isolation step was performed twice to eliminate the possible contamination of JeKo-1 cells. RNA was isolated from the CART19 cells using QIAGEN RNEASY® Plus Mini Kit. RNA was further treated with DNase I (QIAGEN) and purified using RNA Clean & Concentrator (Zymo Research). RNA-seq was performed on an Illumina HTSeq 4000, and raw fastq data files were obtained. The fastq files were viewed in FastQC to check for quality (Cox et al., Mol Ther., 29 (4): 1529-1540, 2020). After verifying the lack of adapter sequences with FastQC, Cutadapt (Martin, EMBnet, 17:10-12, 2011) was used to filter for reads greater than 32 base pairs. Output files were re-checked for quality using FastQC. The latest human reference genome (GRCh38.p13) was downloaded from NCBI. Genome index files were generated using STAR (Cox et al. 2020, supra, and Dobin et al., Bioinformatics, 29:15-21, 2012). Paired end reads from fastq files were mapped to the genome for each condition. HTSeq was used to generate expression counts for each gene (Cox et al. 2020, supra; and Putri et al., Bioinformatics, 38:2943-2945, 2022). DeSeq2 was used to calculate differential expression using p-values (α=0.05) (Cox et al. 2020, supra; and Love et al., Genome Biol., 15:550, 2014). The pheatmap package was used to generate a heatmap and the Enhanced Volcano package was used to create a volcano plot for significantly differentially expressed genes (p-value<0.05). QIAGEN Ingenuity pathway analysis (IPA) software was used to explore the top canonical pathways and top upstream regulators associated with the significantly differentially expressed genes (p-value<0.05) (Krämer et al., Bioinformatics, 30:523-530, 2014). The disruption efficiency of the CRISPR/Cas9 GM-CSF knockout was determined using targeted sequencing through PCR and TIDE analysis (software available at tide.nki.nl) as described elsewhere (Sakemura et al. 2021, supra).

Statistical analysis: Prism Graph Pad (La Jolla, CA, USA) and Microsoft Excel (Microsoft, Redmond, WA, USA) were used to analyze the experimental data. Statistical tests are described in the figure legends. Briefly, normally distributed data were tested by one- and two-way analysis of variance (ANOVA) followed by Dunnett's multiple comparisons test, and unpaired and paired two-sample Student's t-test or Mann-Whitney U test were used for two-group comparisons. Survival was estimated using the Kaplan-Meier curve and Log-rank test was used to test the hypotheses for in vivo survival.

### Results

### Axl is Expressed on CAR T Cells and Differentiated Myeloid Cells

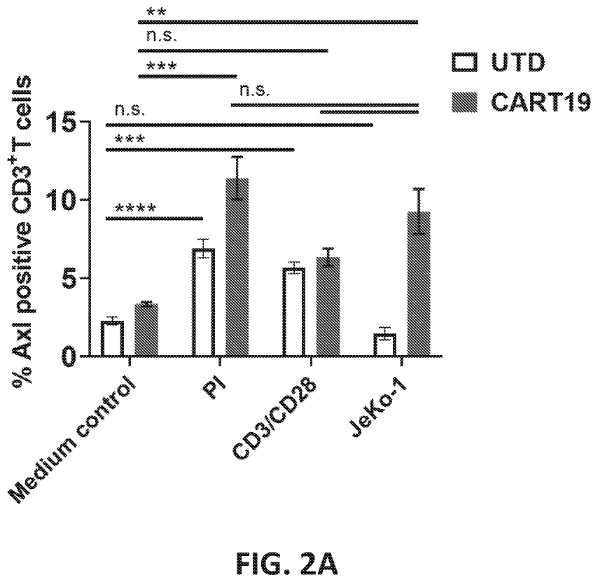

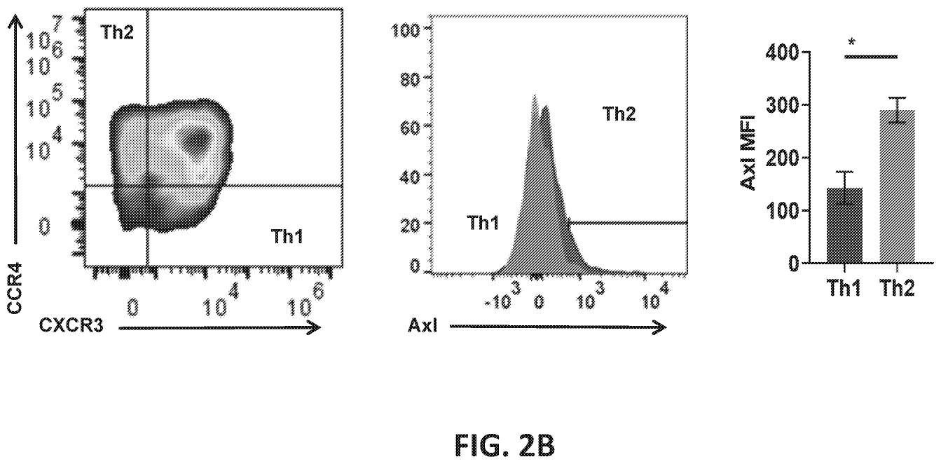

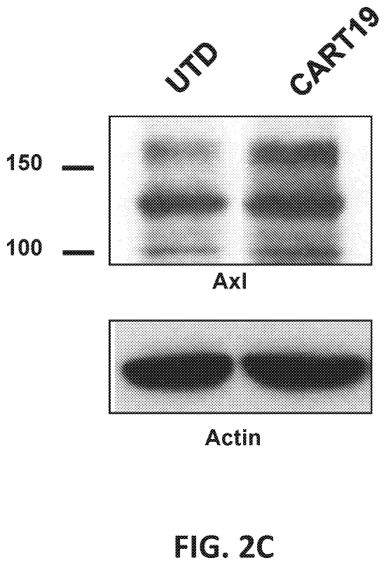

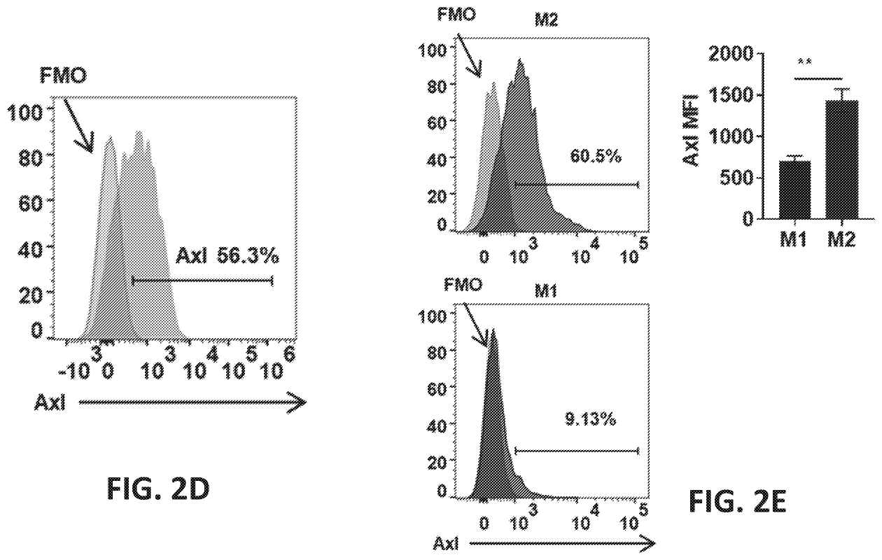

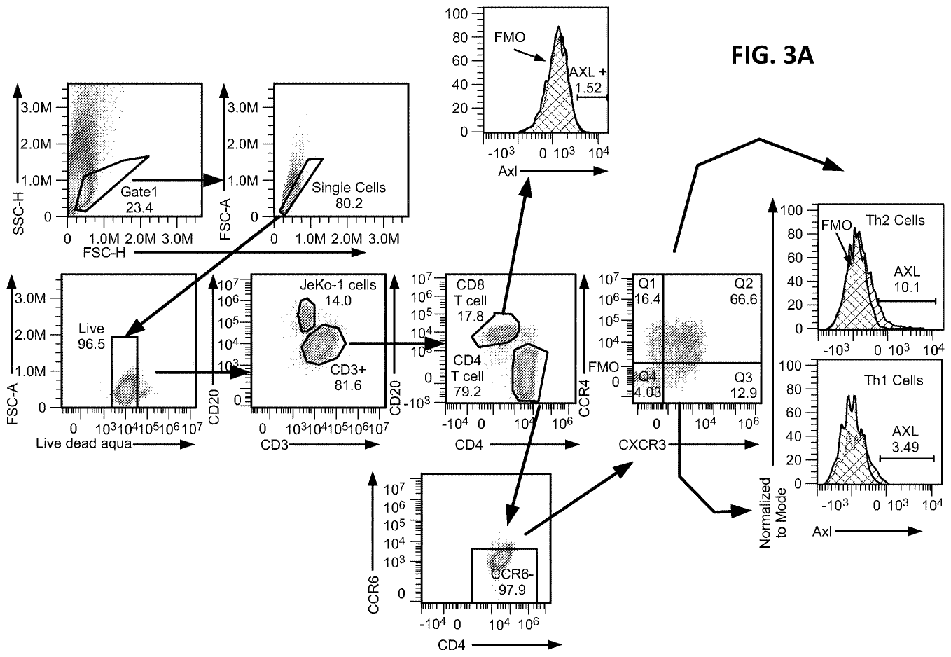

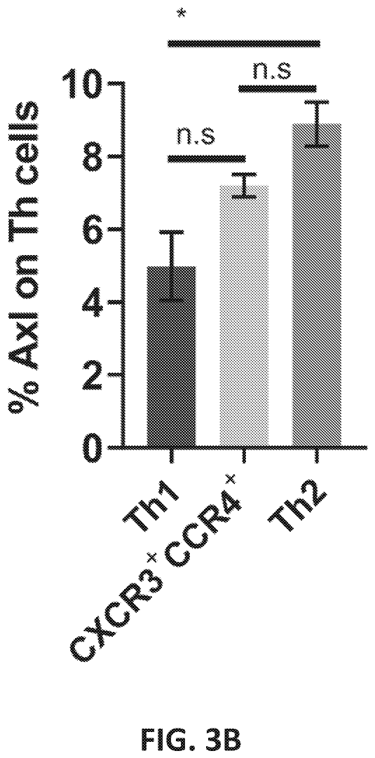

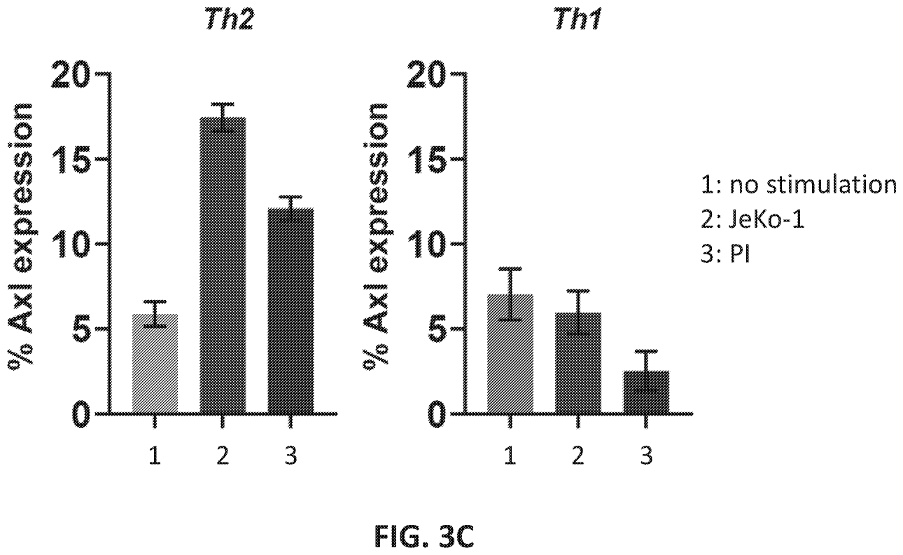

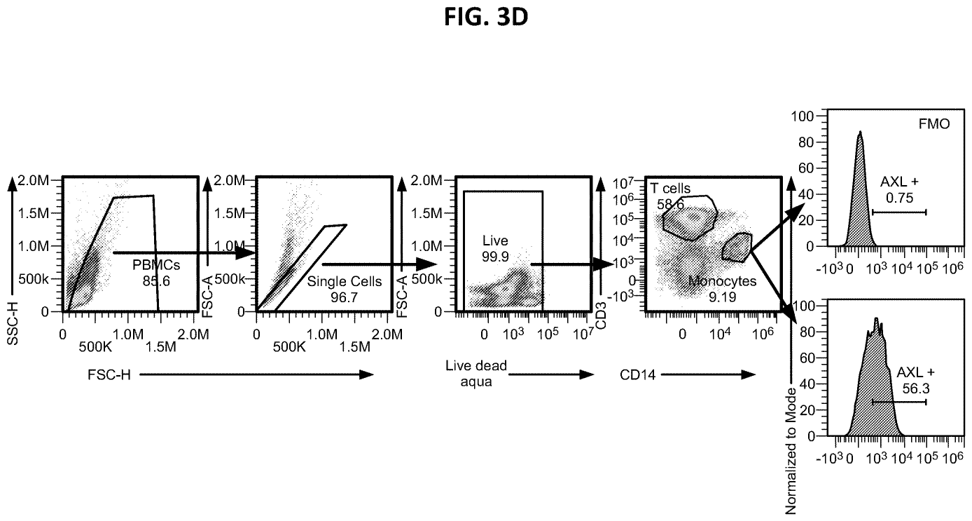

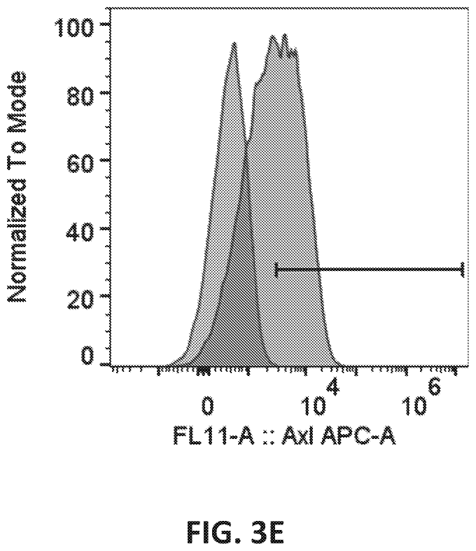

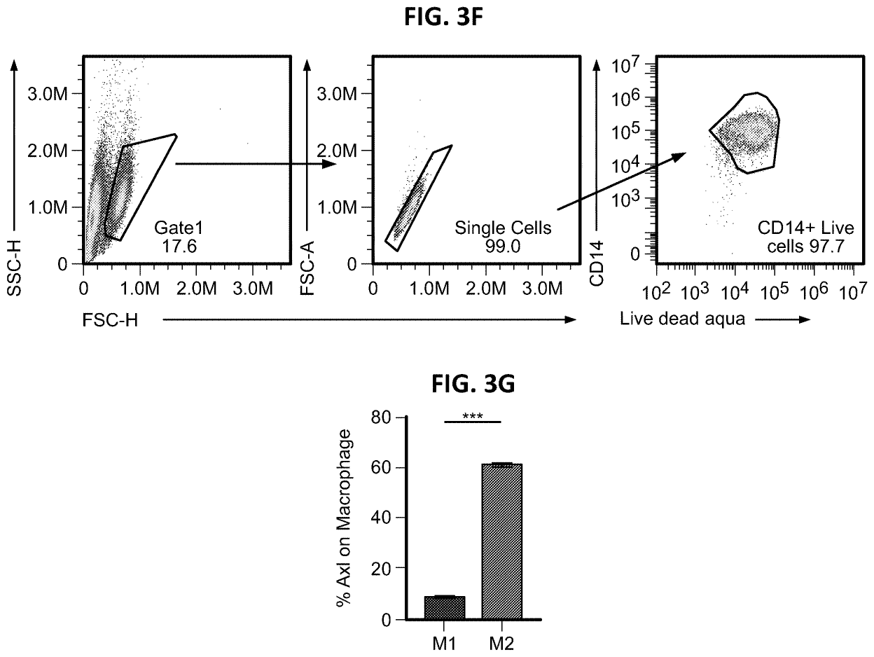

Studies were conducted to assess the expression of Axl on T cell subsets, on CAR T cells, and on activated CAR T cells. The results of these studies indicated that both T cells and CAR T cells express Axl, which is further induced upon activation (FIGS. 2A and 3A). CD19-directed CAR T (CART19) cells that were activated, either non-specifically [via phorbol 12-myristate-13-acetate (PMA) and ionomycin], or via their CAR (by a co-culture with lethally irradiated CD19⁺ mantle cell lymphoma (MCL) cell line JeKo-1), expressed significantly higher levels of Axl, compared to resting CART19 cells, as determined by flow cytometry (FIG. 2A). There was significantly higher Axl expression on Th2 CART19 cells compared to Th1 CART19 after their stimulation (FIGS. 2B, 3B, and 3C). Western blotting confirmed the flow cytometric findings, demonstrating significant expression of Axl on both UTD and CART19 cells (FIG. 2C). Analysis of innate immune cells also revealed significant Axl expression on monocytes (FIGS. 2D and 3D), similar to prior reports (Fujimori et al., Mucosal Immunol., 8:1021-1030, 2015). JeKo-1 cells robustly expressed Axl on their surface (FIG. 3E). Also consistent with studies described elsewhere (Shibata et al., J Immunol., 192:3569-3581, 2014; Zahuczky et al., PLOS One, 6: e21349, 2011; and Waterborg et al., Rheumatol., 58:536-546, 2019), M2-polarized macrophages express higher levels of Axl compared to M1-polarized macrophages (FIGS. 2E, 3F, and 3G).

### Axl Inhibition Selectively Reduces Inhibitory Th2 Cytokines and Prevents the Expansion of Regulatory T Cells

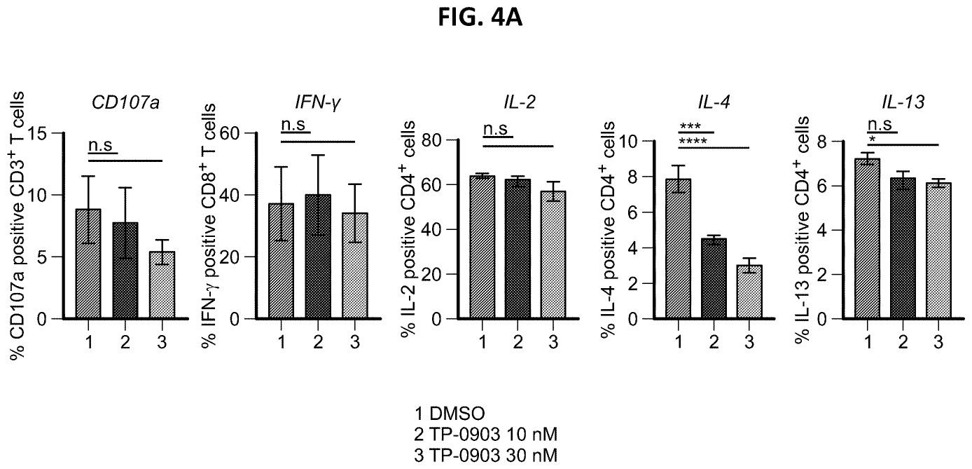

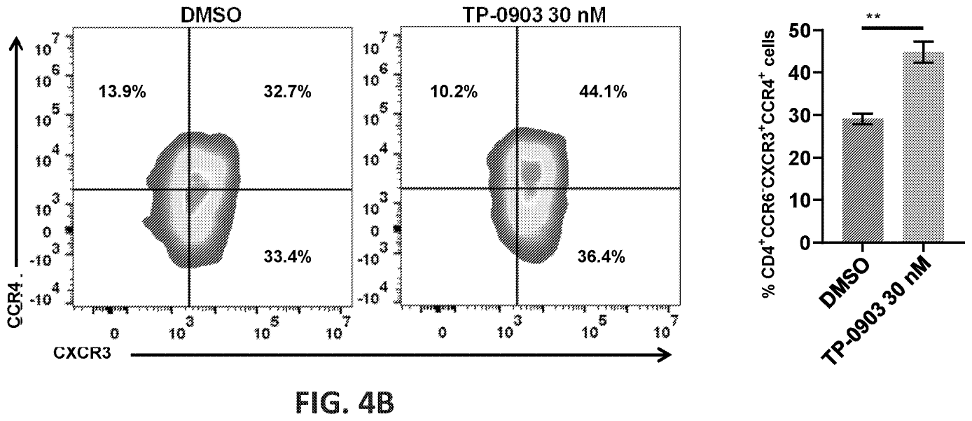

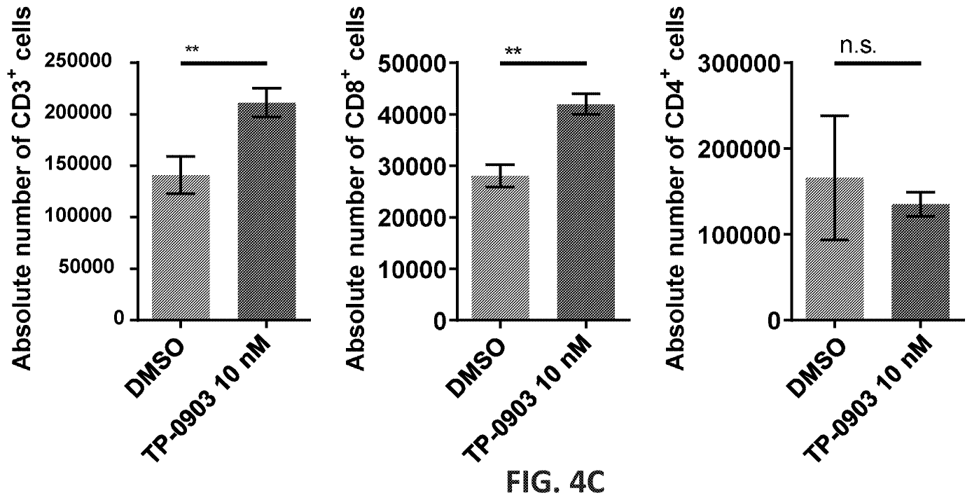

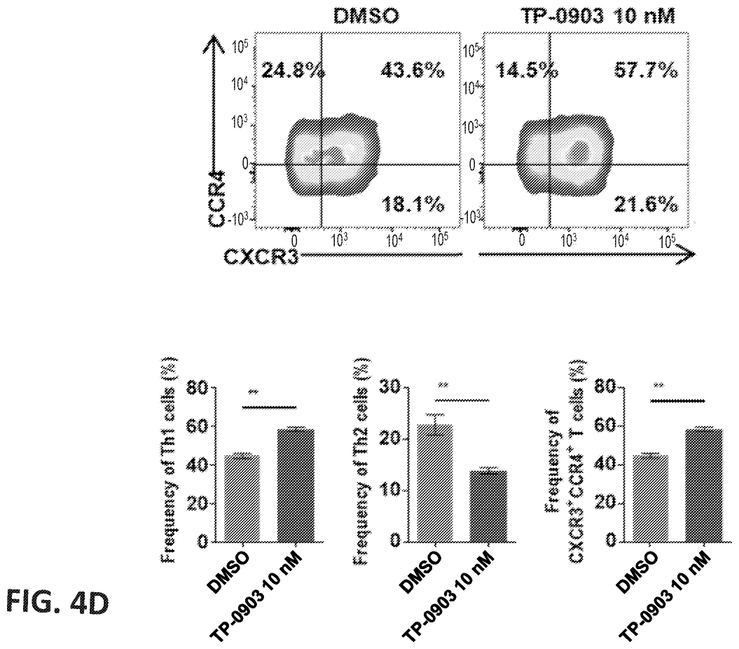

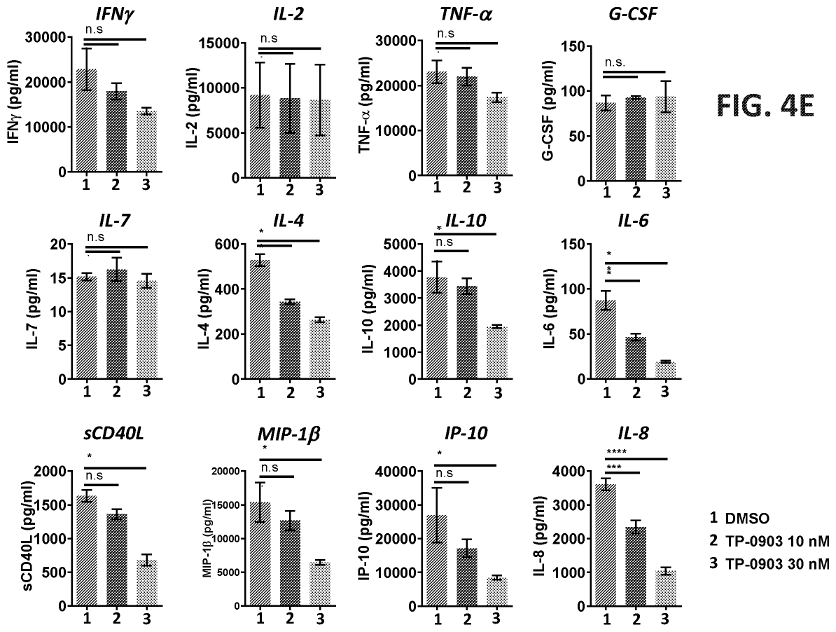

After showing that Axl expression was induced on activated T cells, studies were conducted to determine the effects of Axl inhibition on T cells. When T cells isolated from peripheral blood mononuclear cells (PBMCs) of normal donors were stimulated with PMA (50 ng/mL) and ionomycin (1 μg/mL) in the presence of the highly specific Axl inhibitor TP-0903, there was a significant reduction in the immunosuppressive cytokines, IL-4 and IL-13, while production of Th1 cytokines and effector cytokines (IL-2 and IFN-γ) were preserved (FIG. 4A), suggesting selective targeting of Th2 cells. To further confirm this modulation, T cell immuno-phenotype was determined by flow cytometry. Freshly isolated T cells were stimulated with 5 ng/ml PMA and 0.1 μg/mL ionomycin for 3 days and stained for chemokine receptors, revealing a relative increase of the CCR6⁻CXCR3⁺CCR4⁺ fraction following Axl inhibition (FIG. 4B).

### Axl Inhibition of CAR T Cells Reduces Inhibitory Cytokines and Enhances CAR T Cell Proliferation

Since the experiments described above indicated that Axl inhibition modulated activated T cell phenotype and cytokine production, further studies were aimed at determining whether this effect applies to CART19 cells. The direct anti-tumor effect of Axl inhibition against malignant B cell targets was evaluated using CD19⁺ JeKo-1 cells or leukemic B cells derived from chronic lymphocytic leukemia (CLL) patients by performing an in vitro killing assay. While 65 nM of Axl inhibitor TP-0903 resulted in direct anti-tumor activity, there was no observed killing of tumor cells at lower doses of the Axl inhibtor TP-0903 (10-30 nM) (FIGS. 5A and 5C), although earlier studies had shown that TP-0903 results in potent inhibition of Axl phosphorylation in B cell malignancies at the lower dose levels (Sinha et al., Clin Cancer Res., 21:2115-2126, 2015). To determine the specific effects of Axl inhibition on CART19 cells, independent of its antitumor effect, the lower doses (10-30 nM) of TP-0903 were used for the further experiments described herein.

First, studies were conducted to examine the imapct of Axl inhibition on CART19 effector functions. CART19 cells were stimulated through the CAR with CD19⁺ JeKo-1 cells at a 1:5 ratio and maintained in culture for 5 days. Treatment with 10 nM TP-0903 resulted in superior CD8⁺ CAR T cell proliferation (FIG. 4C). Immunophenotyping of the stimulated CAR T cells by flow cytometry suggested a relative increase in Th1 phenotype following Axl inhibition (Th1 CAR T cells were defined as CD4⁺CCR6⁻CXCR3⁺CCR4⁻ and CD4⁺CCR6⁻CXCR3⁺CCR4⁺ cells, while Th2 CAR T cells defined as CD4⁺CCR6⁻CXCR3⁻CCR4⁺ cells; FIG. 4D, lower panel). The effect of Axl inhibition with TP-0903 on Th2 cells was further confirmed by measuring secreted cytokines 24 hours after CAR T cell stimulation (via co-culture with irradiated JeKo-1 cells) in the presence of TP-0903. Axl inhibition of CAR T cells resulted in a reduction in IL-4, IL-10, IL-6, sCD40L, MIP-1β, IP-10, and IL-8 but not IFN-γ, IL-2, TNF-α, and IL-7 (FIG. 4E).

The lack of anti-tumor activity with low doses (10-30 nM) TP-0903 (FIGS. 5A and 5B) suggested that these observations were related to direct effects on T cells, rather than a direct anti-tumor effect. These results were consistent when experiments were repeated using a different CD19⁺ cell line or patient-derived CD19⁺ CLL cells in a co-culture with CART19 cells (FIGS. 5C and 5D). Given the well-known difficulty in maintaining primary, patient-derived malignant CD19⁺ B cells in culture and the consistency of observed trends across CD19⁺ target cell types, the CD19⁺ JeKo-1 MCL cell line was used in the further experiments described below.

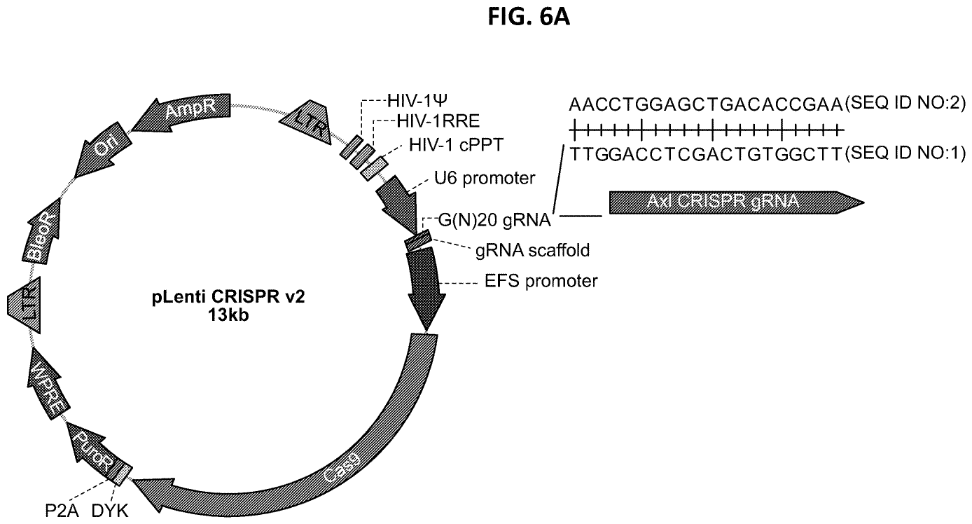

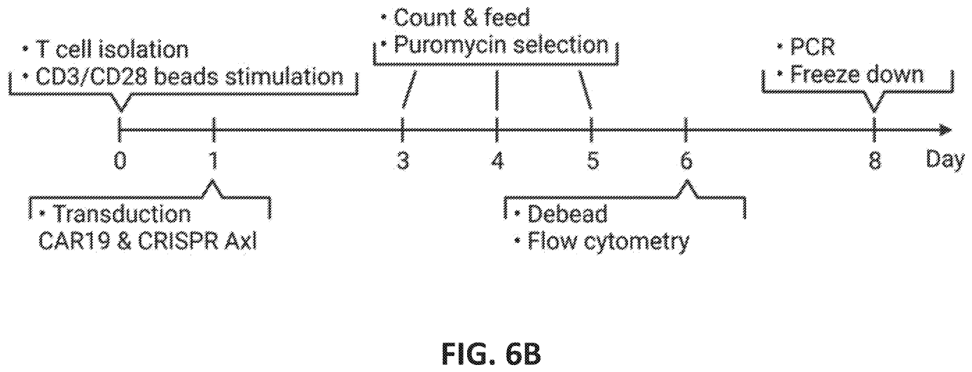

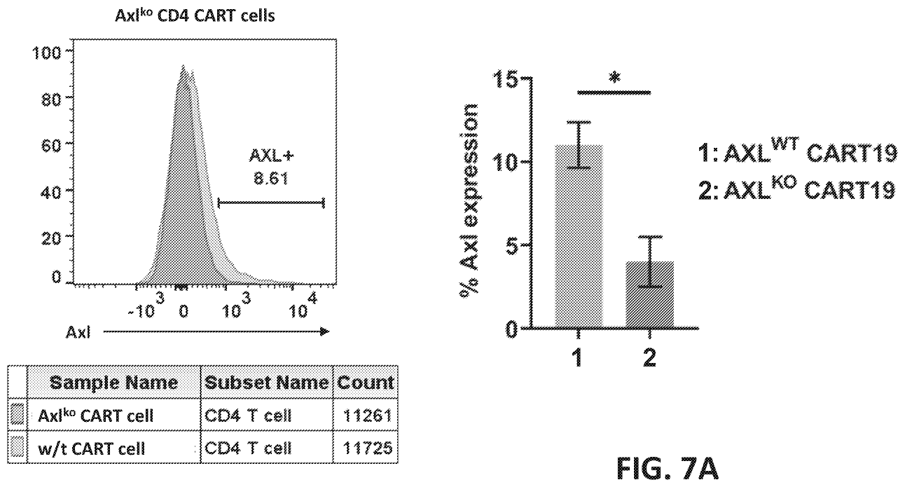

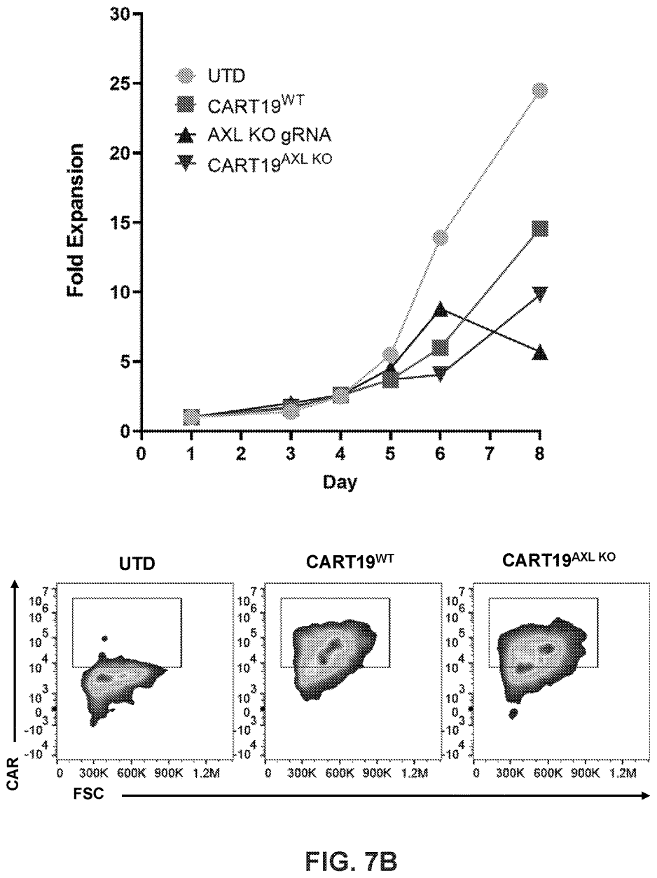

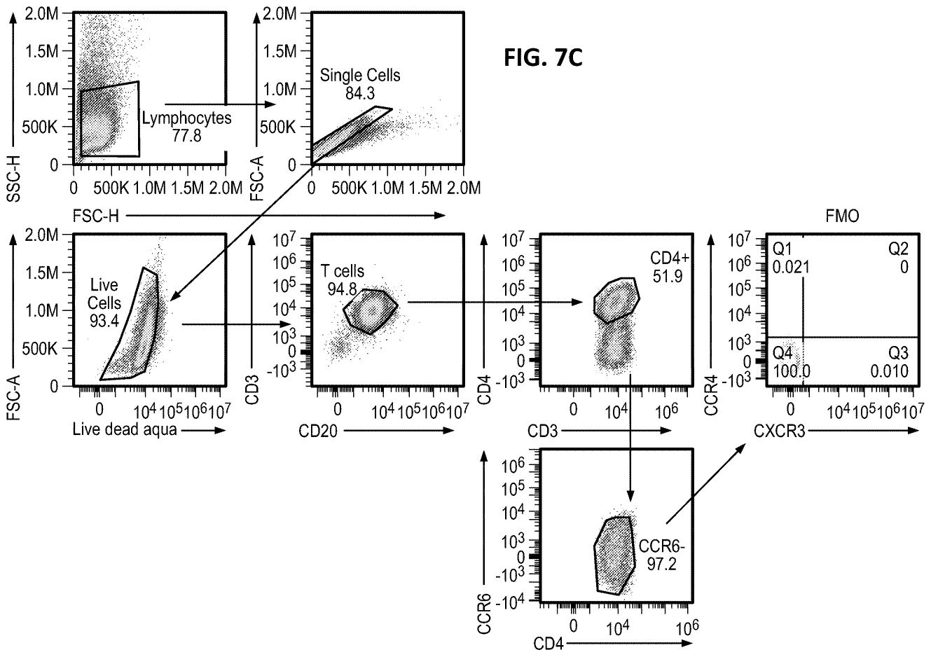

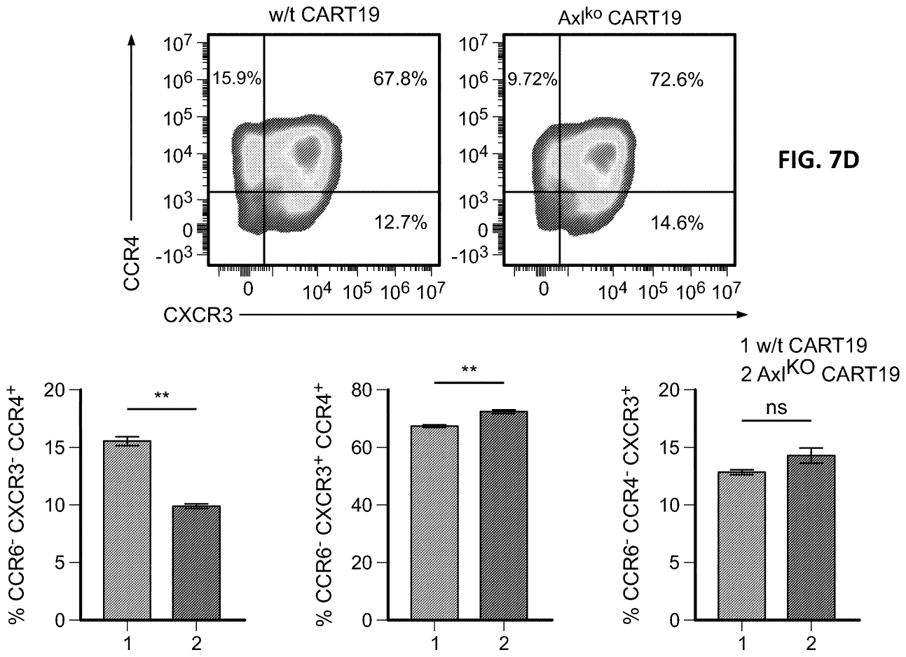

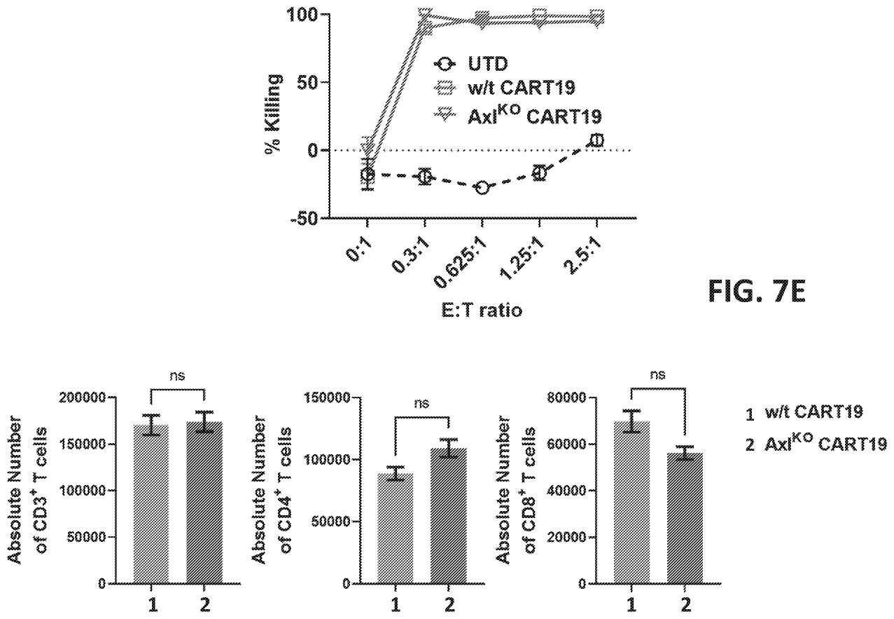

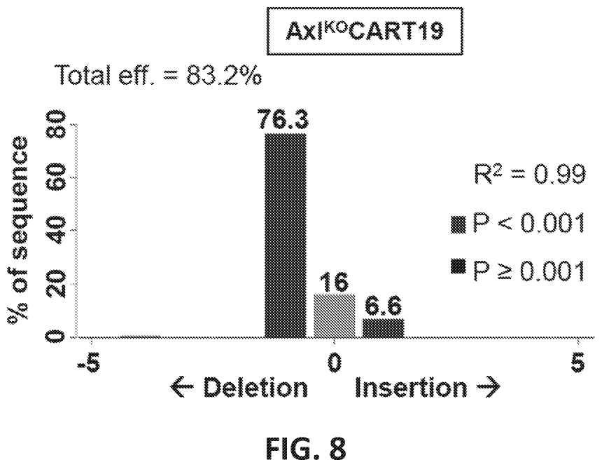

TP-0903 has been reported to have off target effects beyond its inhibition of Axl-RTK (Holland et al., Cancer Res. 70:1544-1554, 2010; and Myers et al., J Med Chem, 59:3593-3608, 2016). To confirm that CAR T cell modulation induced by TP-0903 was due to Axl inhibition, the Axl gene was knocked out in CAR T cells during their manufacture using the CRISPR/Cas9 system with a nucleic acid encoding a guide RNA having the sequence set forth in SEQ ID NO:1 (Axelrod et al., supra), cloned into a CRISPR lentivirus backbone (Sterner et al. 2019a, supra), as shown in FIGS. 6A and 6B. Axl was successfully knocked out from CART19 cells (FIG. 7A), with a knockout efficiency of 73.2% (n=3, 67.9-83.2%, FIG. 8). Control CART19 (Axl^{wt} CART19) cells were generated using a CRISPR/Cas9 with a control scrambled guide RNA as described elsewhere (Cox et al. 2022, supra). Knocking out Axl did not impact CAR T cell expansion (FIG. 7B). T cell phenotype and functions of Axl knocked out CART19 (Axl^{k/o} CART19) cells were then evaluated. Control CART19 (Axl^{wt} CART19) or Axl^{k/o} CART19 cells were co-cultured with JeKo-1 cells for 3 days, and T cell phyenotype was assessed via flow cytometry. Similar to Axl inhibition with TP-0903, Axl^{k/o} CART19 showed significant reduction of Th2 and increase in Th1 subsets (FIGS. 7C and 7D). Disruption of Axl in CART19 cells did not impair their immediate functions in vitro (FIGS. 7E and 7F).

Axl Inhibition with TP-0903 Improves Anti-Lymphoma Activity and CAR T Cell Expansion In Vivo

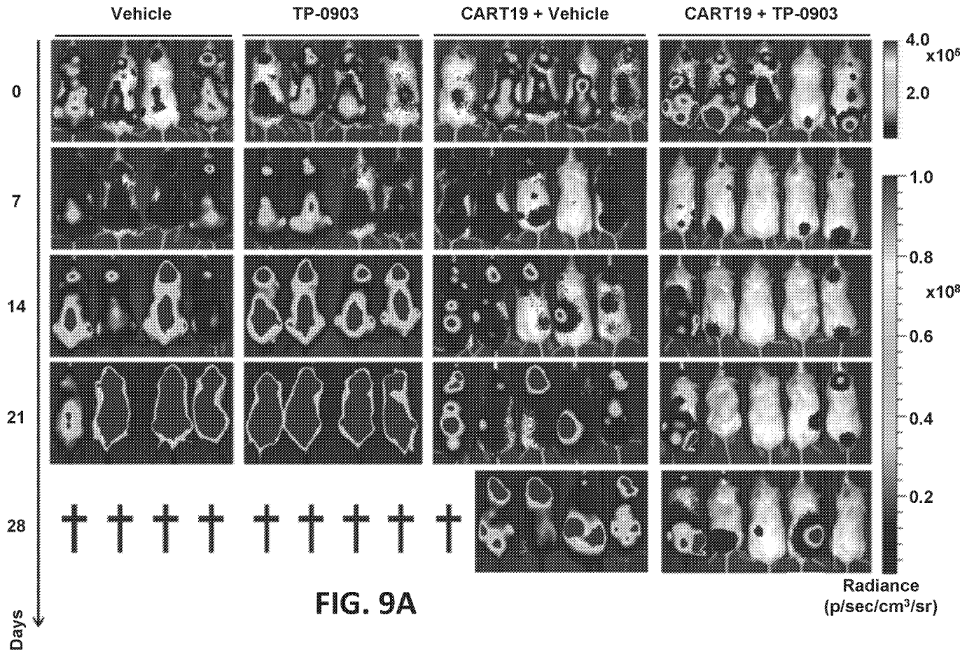

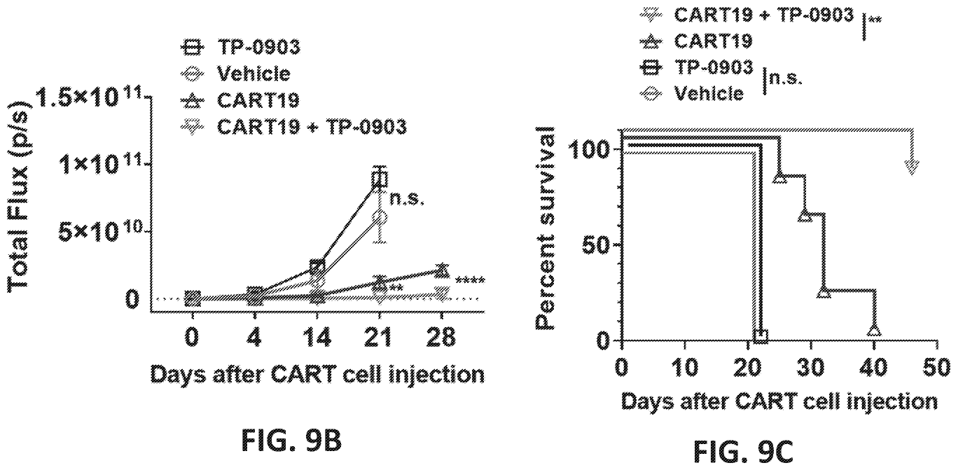

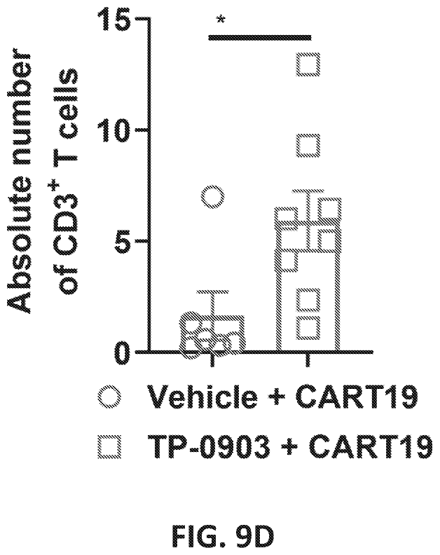

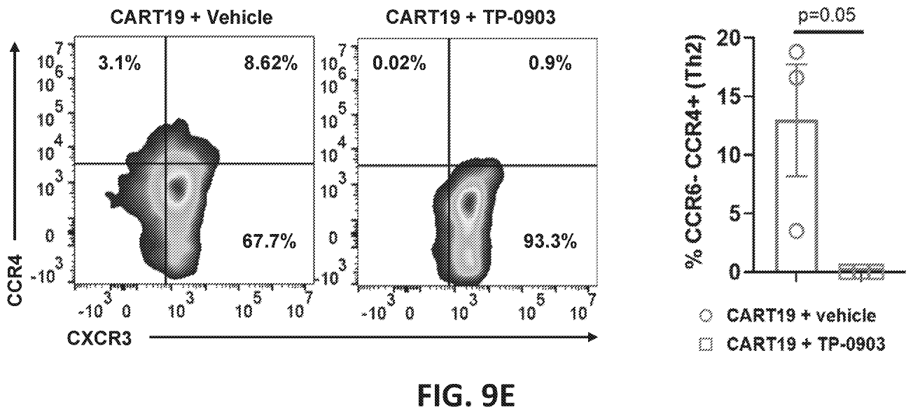

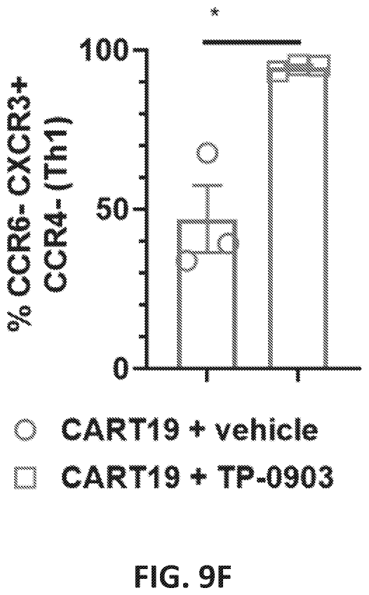

To further validate the impact of Axl inhibition with TP-0903 on CAR T cells in vivo, a JeKo-1-xenograft NOD-SCID-γ^{-/-} (NSG) mouse model was used. TP-0903 and CART19 combination therapy was tested in a JeKo-1 relapse mouse model with higher tumor burden. In these studies, luciferase⁺ JeKo-1 cells (1.0×10⁶) were intravenously injected into NSG mice. Engraftment was confirmed 14 days after the implantation of JeKo-1 cells, and tumor burden was assessed with bioluminescent imaging (BLI). Mice were then randomized based on their BLI to receive control vehicle, a low dose of Axl inhibitor TP-0903 (20 mg/kg/day, which is equivalent to 30 nM in vitro) monotherapy, CART19 (0.5×10⁶ cells) monotherapy, or combination TP-0903 (20 mg/kg/day) and CART19 (0.5×10⁶ cells). TP-0903 was given orally to mice until study completion. The TP-0903 and CART19 combination therapy resulted in superior anti-tumor activity (FIGS. 9A and 9B) and significantly longer overall surivival compared to CART19 monotherapy (FIG. 9C; hazard ratio (HR)=0.089 with 95% confidence interval (CI) (0.01595 to 0.5072), p=0.004). Mice underwent peripheral blood sampling 17 days after CART19 administration, and the amount of circulating CART19 cells in peripheral blood was measured. The combination of TP-0903 and CART19 cells resulted in enhanced CART19 expansion compared to CART19 monotherapy (CART19⁺ vehicle; FIG. 9D). To assess CART19 cell phenotype, satellite mice were euthanized at day 17 of CART19 cell treatment and spleens were harvested. Flow cytometric analysis of splenocytes revealed significant Th1 polarization of CART19 cells in mice treated with TP-0903 and CART19 cells as compared to the CART19 monotherapy group (FIGS. 9E and 9F). The Axl inhibitor TP-0903 monotherapy did not have any significant antitumor activity at the low doses used in this model, further indicating that the significantly enhanced anti-tumor activity of CART19 was a result of direct modulation of CART19 cells by TP-0903.

### Myeloid Cells are Sensitive to Killing by the Axl Inhibitor TP-0903

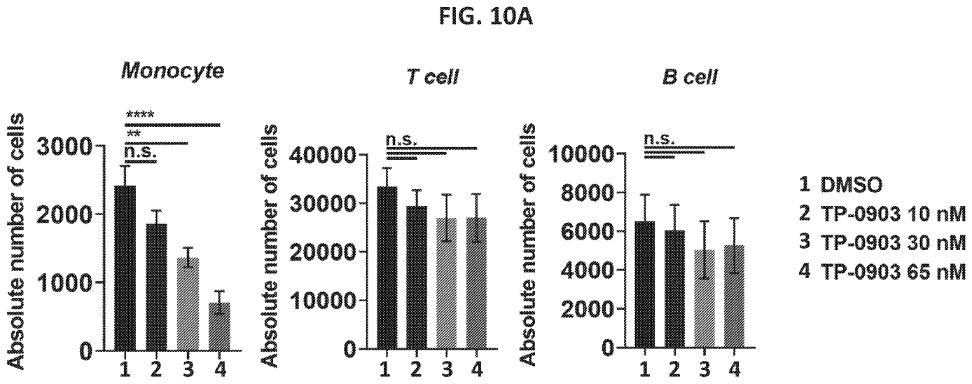

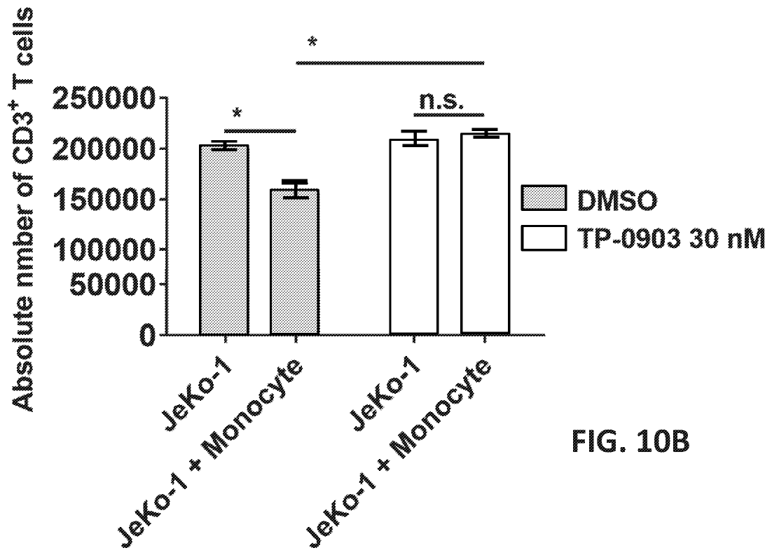

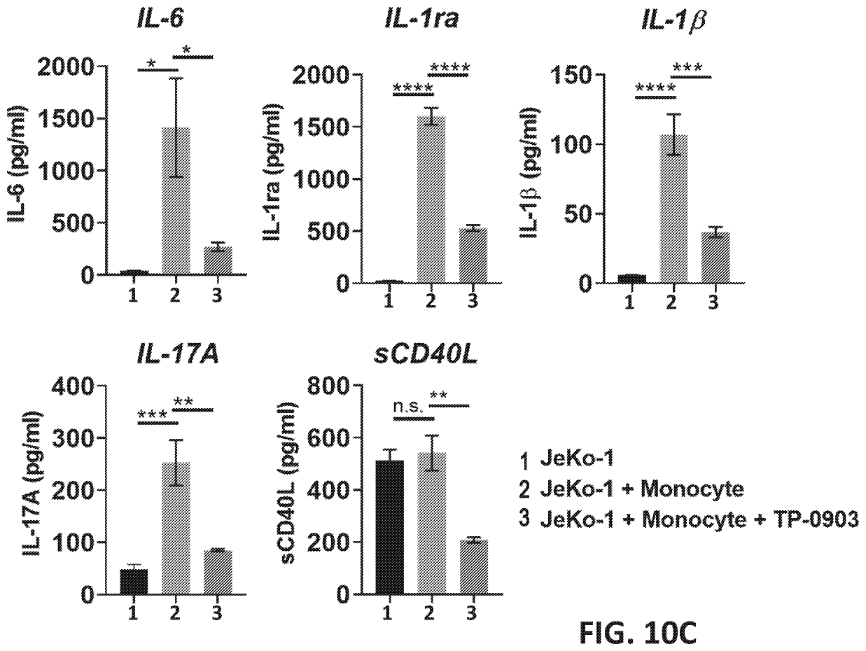

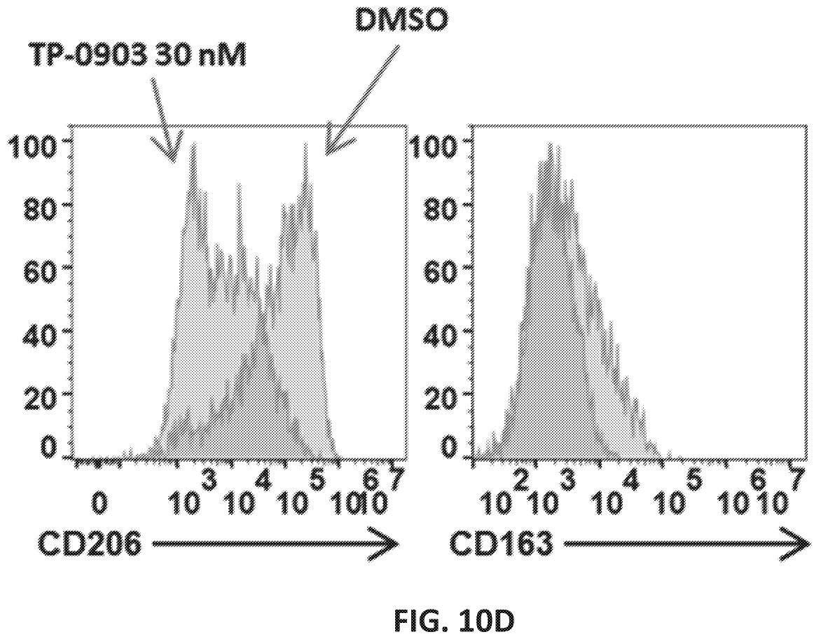

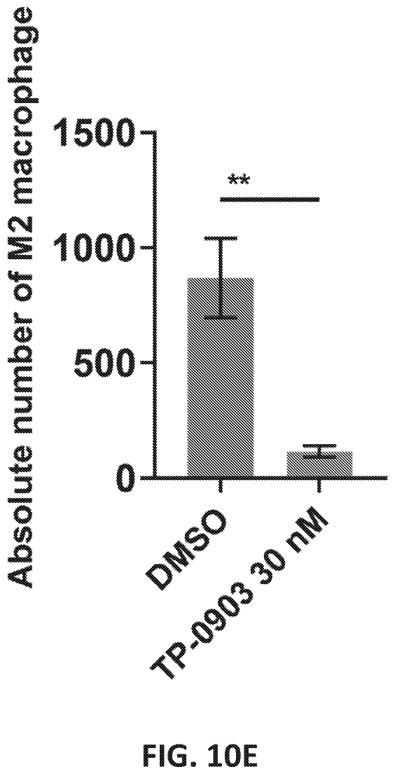

Given the significant upregulation of Axl on CD14⁺ monocytes and M2-polarized macrophages, experiments were performed to determine if there were any functional effects of Axl inhibition on monocytes. Freshly isolated PBMCs derived from healthy donors were treated with various concentrations of TP-0903 or DMSO vehicle control for 24 hours. Compared to T cells, monocytes were significantly more sensitive to TP-0903 vs. the control vehicle at all concentrations tested, as determined by a reduction in their survival measured by flow cytometry (FIG. 10A). These results suggested that, in addition to the direct effects of Axl inhibition on Th2 cells and their secreted cytokine profile, TP-0903 inhibition of Axl has a profound and direct activity on monocytes.

Axl Inhibition with TP-0903 Ameliorates Monocyte-Induced CART19 Cell Inhibition

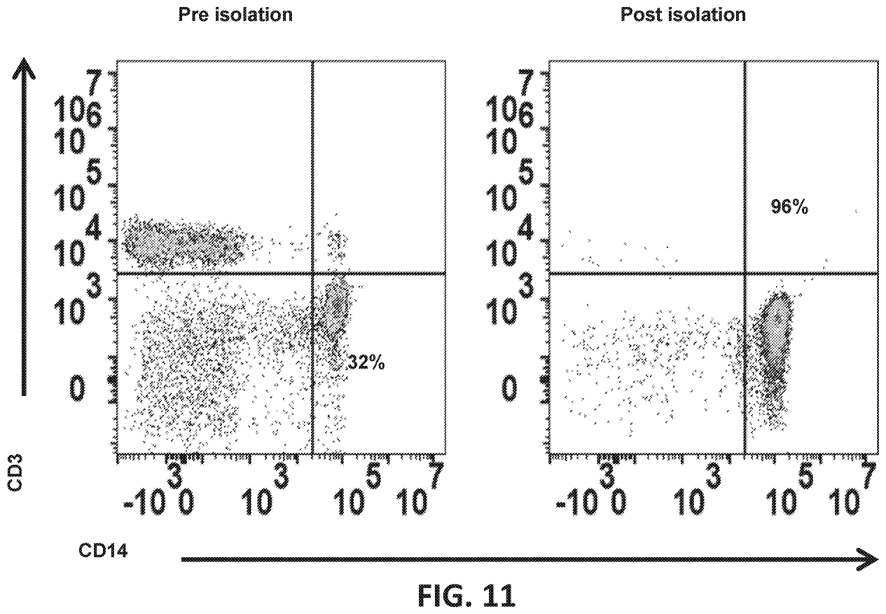

Monocytes and myeloid-derived cytokines can inhibit CAR T cell functions in vitro and in vivo (Stroncek et al., Cytotherapy, 18:893-901, 2016; Norelli et al., Nat Med., 24:739-748, 2018; and Sterner et al. 2019b, supra). Given the observed effect of Axl inhibition on monocytes as described above, further studies were conducted to assess whether Axl inhibition ameliorates monocyte-induced CAR T cell inhibition. Normal healthy donor CART19 cells were cultured with CD19⁺ JeKo-1 cells in the presence of monocytes at a ratio of 1:5:1 (CAR T: monocytes: tumor cells), as inhibition of CAR T cells at this ratio can be achieved (Sterner et al. 2019a, supra; and Sterner et al. 2019b, supra). Monocytes were isolated from PBMCs by CD14⁺ magnetic separation to a high purity (>95% purity; FIG. 11). Co-culture of the CAR T: monocyte: tumor cell mixture was performed in the presence of TP-0903 (30 nM) or vehicle control. At day 5 of the co-culture, cells were harvested and absolute numbers of T cells were counted via flow cytometry. Consistent with findings described elsewhere (Ruella et al., Cancer Discovery, 7:1154-1167, 2017), there was a significant inhibition of CART19 cell proliferation in the presence of monocytes, but this was reversed when TP-0903 was added to the co-culture (FIG. 10B). Cytokine analysis of supernatant harvested 72 hours after the monocyte/TP-0903 co-culture demonstrated significant reductions of myeloid-related cytokines, including IL-6, IL-1 receptor α, IL-1β, IL-17A, and soluble CD40 ligand in the presence of low doses of TP-0903 (FIG. 10C). This suggested a direct effect of TP-0903 on monocyte function. In addition, flow cytometric analysis of myeloid cell subsets following this co-culture suggested a selective reduction in M2 macrophages (FIGS. 10D and 10E).

### TP-0903 Inhibition of CART19 Cells is Specific for Axl

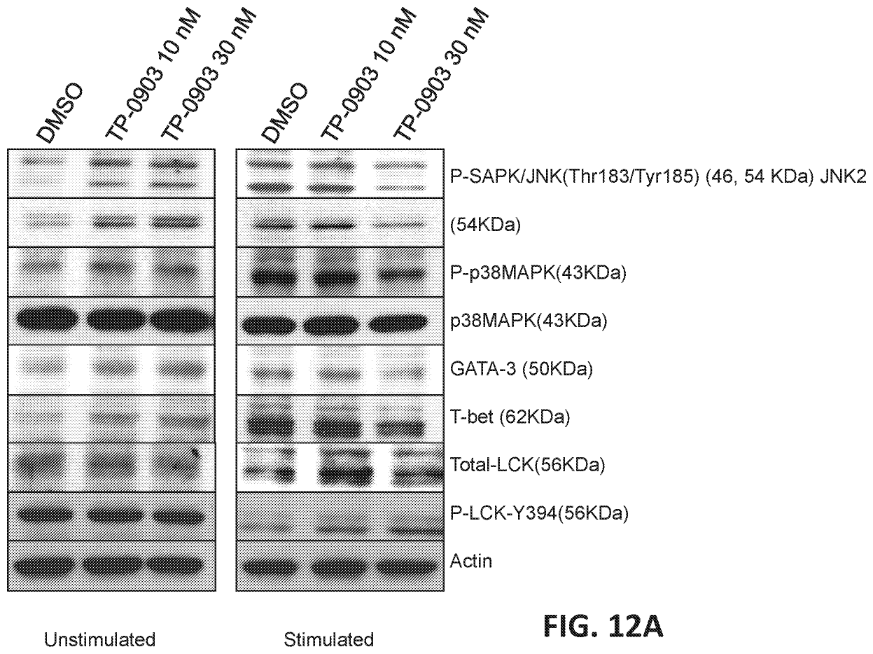

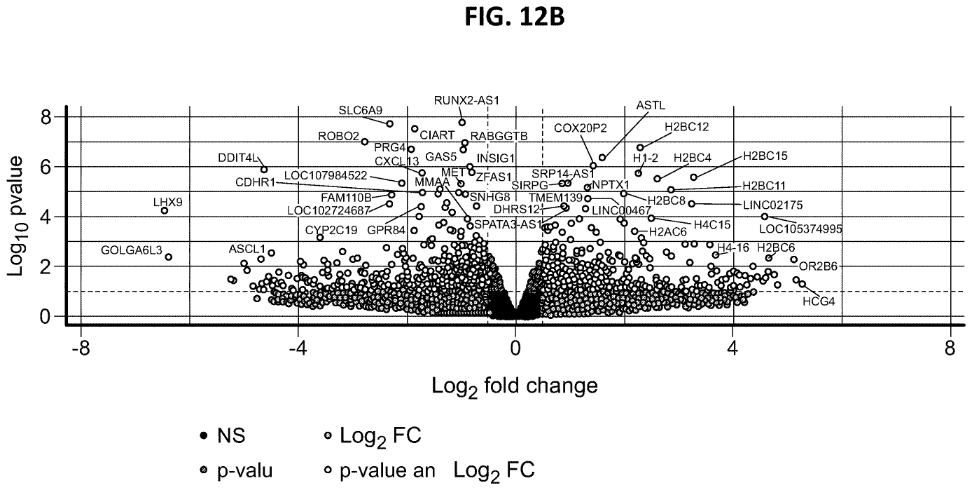

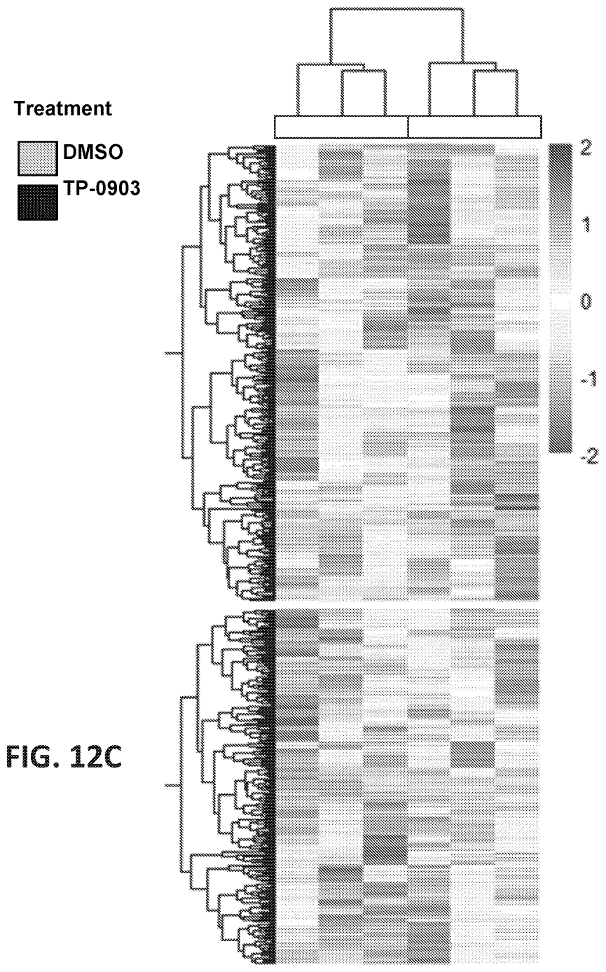

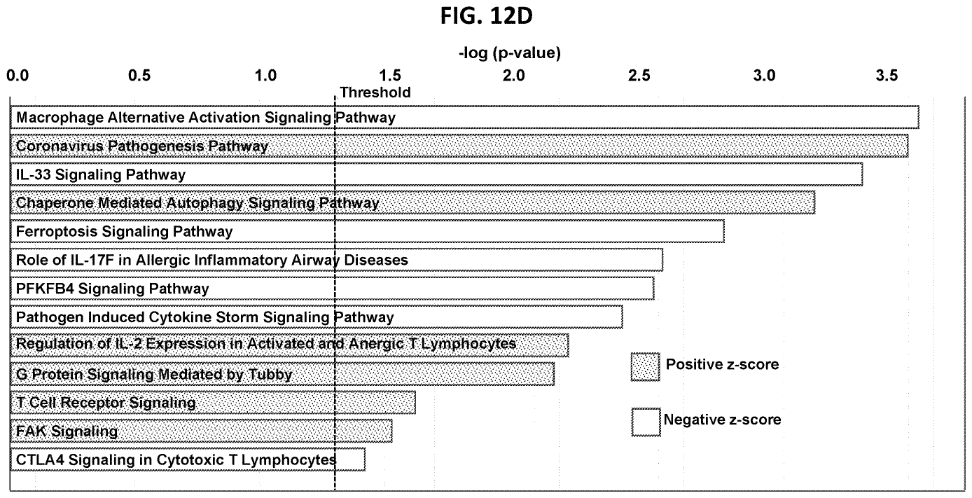

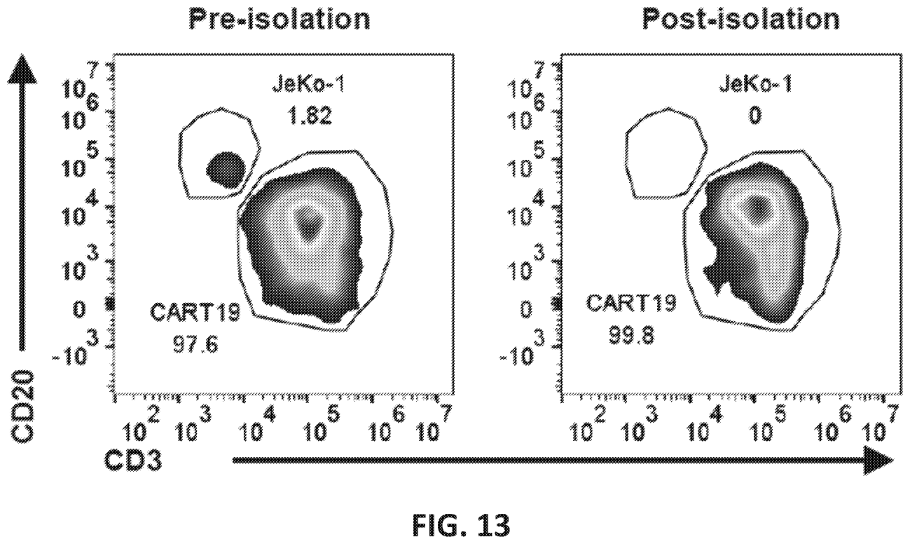

As discussed above, Axl expression is upregulated on activated Th2 and M2 cells, Axl inhibition reduces inhibitory cytokines and synergizes with effector T cells and CART19 cells, and Axl inhibition ameliorates myeloid cell-induced T cell inhibition. Additional studies were conducted to confirm that the observed effects were due to the selective killing of Th2 cells and M2 cells, and not due to off-target effects by TP-0903. First, downstream signaling through Axl and other potential non-Axl targets for TP-0903 was interrogated. There were no changes in phosphorylation of JNK, p38, GATA-3, T-bet, or LCK of CART19 cells following treatment with low doses (10-30 nM) of TP-0903 (FIG. 12A). CART19 transcriptome changes following Axl inhibition were then evaluated by performing RNA sequencing of activated CART19 cells after treatment with 30 nM TP-0903. CART19 cells were stimulated with CD19⁺ JeKo-1 cells in the presence of the low-dose TP-0903 or vehicle control for 24 hours. Subsequently, the JeKo-1 cells were depleted using CD4⁺ and CD8⁺ magnetic sorting, and flow cytometric analysis confirmed>99% purified T cell fractions after the isolation (FIG. 13). RNA sequencing of CART19 cells was then performed. These studies revealed that there were 322 significantly upregulated and 414 significantly downregulated genes after treatment with 30 nM TP-0903 (FIGS. 12B and 12C). Data were analyzed with the use of QIAGEN Ingenuity Pathway Analysis (IPA) (digitalinsights.qiagen.com/IPA; Krämer et al., Bioinformatics 30 (4): 523-530, 2014). This analysis identified the Macrophage Alterative Activation Signaling Pathway as the most significantly altered pathway following Axl inhibition with TP-0903 (Martinez et al., Annu Rev Immunol. 27:451-483, 2009) (FIG. 12D). This pathway is associated with immune-suppressing signals, including CXCL13 (Tokunaga et al., Cancer Treat Rev., 63:40-47, 2018; and Xie et al., Cancer Cell Int., 21:677, 2021), IL-4, and IRF4 (Man et al., Immunity 47:1129-1141.e5, 2017). In addition, the IL-33 Signaling Pathway was significantly suppressed when CART19 cells were treated with TP-0903, indicating inhibition of the Th2 mediated response (Pinto et al., J Cell Commun Signal., 12:615-624, 2018; Schmitz et al., Immunity, 23:479-490, 2005; Rak et al., J Invest Dermatol., 136:487-496, 2016; and Monticelli et al., Proc Natl Acad Sci USA, 112:10762-10767, 2015). Consistent with the findings of interactions between CART19 and myeloid cells, as shown in FIGS. 10A-10E, pathway analysis identified the Role of IL-17F in Allergic Inflammatory Airway Diseases and Pathogen Induced Cytokine Storm Signaling pathway (Mills, Nat Rev Immunol. 23 (1): 38-54, 2023; Akira and Takeda, Nat Rev Immunol, 4 (7): 499-511, 2004; and Fajgenbaum and June, N Engl J Med, 383 (23): 2255-2273, 2020) as being significantly altered following Axl inhibition with TP-0903. Axl inhibition also significantly promoted the Regulation of IL-2 Expression in Activated and Anergic T Lymphocytes pathway (Waugh et al., Vaccines (Basel), 3 (3): 771-802, 2015), the T Cell Receptor Signaling pathway (Gaud et al., Nat Rev Immunol, 18 (8): 485-497, 2018), and the G Protein Signaling Mediated by Tubby pathway (Santagata et al., Science, 292 (5524): 2041-2050, 2001). The top five upstream regulators of differentially expressed genes, identified with QIAGEN IPA, were IL-4, TNF, CSF2, TGF-β, and SATB1 (TABLE 5). Datasets with a p-value of 0.001 or less also are listed in TABLE 6.

TABLE 5

| Upstream | Predicted | Activation | p-value |
|---|---|---|---|
| Regulator | Activation State | z-score | of overlap |
| IL-4 | Inhibited | −3.271 | 9.26E−13 |
| TNF | Inhibited | −2.543 | 1.36E−10 |
| CSF2 | Inhibited | −2.255 | 7.83E−10 |
| TGF-β | Inhibited | −2.031 | 7.08E−08 |
| SATB1 | | −1.359 | 2.01E−08 |

TABLE 6

| | | | Predicted | | | | |
|---|---|---|---|---|---|---|---|
| Upstream | Expr Log | Molecule | Activation | Activation | | P-Value of | |
| Regulator | Ratio | Type | State | Z-Score | Flags | Overlap | Target Molecules in Dataset |
| IL4 | −1.068 | cytokine | inhibited | −3.271 | | 9.26E−13 | ACTA2, AICDA, ALDH2, AREG, ARG2, |
| | | | | | | | ATP1A3, BCL11A, BNIP3, BPGM, |
| | | | | | | | CAMK1D, CARD16, CD1C, CD2, CD22, |
| | | | | | | | CD300A, CD69, CD79A, CD8A, CEBPB, |
| | | | | | | | COL3A1, CXCL10, DNAJC12, EPHA2, |
| | | | | | | | FCER2, FN1, GNA14, GPR84, H2BC5, |
| | | | | | | | HILPDA, HK2, HNRNPH3, HOMER2, |
| | | | | | | | IGF2R, IL13RA1, IL16, IL3, IL4, IL5, |
| | | | | | | | IL5RA, IL9, IRF4, ITGB3, JCHAIN, KMO, |
| | | | | | | | LACC1, LIPG, MAOA, MIR-29, MPEG1, |
| | | | | | | | MS4A1, NFKBIZ, PIGR, PODXL, PRKAG1, |
| | | | | | | | RPS6, SELL, SERPINB9, SH2D1B, SPP1, |
| | | | | | | | STC2, TGM2, TSLP, VEGFA |
| TNF | | cytokine | inhibited | −2.543 | | 1.36E−10 | A2M, ACTA2, AICDA, ALDH2, |
| | | | | | | | APOBEC3B, ATF4, BDNF-AS, BPGM, |
| | | | | | | | CARD16, CCR2, CD1C, CD69, CEBPB, |
| | | | | | | | CLCF1, COL27A1, COL3A1, CREM, |
| | | | | | | | CXCL10, CXCL13, DAXX, DLEU1, EMP1, |
| | | | | | | | EPHA2, FCER2, FN1, GADD45A, GAS5, |
| | | | | | | | GPR84, GTDC1, HK2, IGKC, IL16, IL3, IL4, |
| | | | | | | | IL5, IL9, INSIG1, IRF4, ITGB3, JAG2, |
| | | | | | | | KLF7, KMO, MET, MMP21, NFKBIZ, NID1, |
| | | | | | | | NLRP3, NOXO1, NQO1, NR4A2, OTUD7B, |
| | | | | | | | PDPN, PHGDH, PHLDA1, PIGR, PODXL, |
| | | | | | | | RASD1, RASGRF1, RNF125, RPS3, RPSA, |
| | | | | | | | RRM2, S1PR3, SCD, SELL, SERPINB9, |
| | | | | | | | SFRP1, SGPL1, SLAMF8, SLC14A1, SPP1, |
| | | | | | | | TG, TGM2, TINCR, TLR7, TNFSF15, TPT1, |
| | | | | | | | TRAF6, TRPC1, TSC22D3, TSLP, VEGFA |
| CSF2 | −0.066 | cytokine | inhibited | −2.255 | | 7.87E−10 | ACTA2, BATF3, CARD16, CCR2, CD180, |
| | | | | | | | CD1C, CD69, CD8A, CLCF1, CXCL10, |
| | | | | | | | FANCL, HILPDA, HK2, IL16, IL4, IL5, |
| | | | | | | | IL5RA, IRF4, ITGB3, LY9, MET, NCAPH, |
| | | | | | | | NDST1, NLRP3, NR4A2, PHGDH, RBM3, |
| | | | | | | | RRM2, SERPINB9, SPP1, TGM2, TNFSF15, |
| | | | | | | | TPM4, TPT1 |
| TGF BETA | | group | inhibited | −2.031 | | 1.08E−08 | ACTA2, ARID3C, ASCL1, ASCL4, BNIP3, |
| | | | | | | | CCR2, CD69, CD79A, CEBPB, COL3A1, |
| | | | | | | | CXCL10, EID3, FN1, GPR55, GRID2IP, |
| | | | | | | | IL16, IL4, IL5, IL9, ITGB3, LEPR, MET, |
| | | | | | | | MPEG1, NQO1, NR4A2, PFKFB4, SMAD2, |
| | | | | | | | SPP1, TSLP, VEGFA |
| SATB1 | −0.104 | transcription | | −1.359 | | 2.01E−08 | BLNK, CD69, CD8A, CLEC2B, GPT2, IL16, |
| | | regulator | | | | | IL3, IL4, IL5, NR4A2, RAG1, RPLP0, SELL, |
| | | | | | | | SIPA1L2, TSC22D3, TSLP, YPEL5 |
| HRAS | | enzyme | | −1.187 | | 2.26E−08 | A2M, AREG, ASCL1, ATP1A3, BNIP3, |
| | | | | | | | CALD1, CD69, CD96, CEBPB, CLCF1, |
| | | | | | | | COL3A1, COL5A2, CXCL10, FABP3, FN1, |
| | | | | | | | GNA14, IGKC, IL13RA1, LIPG, MET, NID1, |
| | | | | | | | NQO1, NUCKS1, PDGFRB, PDLIM1, RPS3, |
| | | | | | | | RPS6, RPS6KA2, RRM2, SCD, SERPINB9, |
| | | | | | | | SPP1, TG, TGM2, TSLP, TSPAN6, VEGFA |
| POU2F2 | −0.143 | transcription | | −1 | bias | 3.35E−08 | ARG2, BANK1, CD22, CD69, FCRL2, FJX1, |
| | | regulator | | | | | H2BC21, IGKC, IL5RA, MS4A1, PFKFB4, |
| | | | | | | | SCD, SPP1 |
| LARP1 | −0.22 | translation | activated | 3.461 | bias | 5.47E−08 | EEF1A1, EIF4B, HNRNPA1, RPL31, RPS12, |
| | | regulator | | | | | RPS15A, RPS23, RPS3, RPS3A, RPS4X, |
| | | | | | | | RPS6, TPT1 |
| EPAS1 | −0.784 | transcription | | −0.365 | | 9.84E−08 | AREG, ARG2, BNIP3, CCR2, CEBPB, |
| | | regulator | | | | | CYP51A1, EOLA1/EOLA2, FN1, GPT2, H1- |
| | | | | | | | 2, H2AC6, HILPDA, ITGB3, MYO7A, |
| | | | | | | | PFKFB4, SNHG5, STC2, TMEM45A, |
| | | | | | | | TNFSF15, VEGFA |
| MYCN | −0.217 | transcription | inhibited | −2.592 | | 0.000000114 | BCL11A, COL5A2, DIRAS1, EEF1A1, |
| | | regulator | | | | | EMP1, FN1, HK2, MIR-29, NTRK1, |
| | | | | | | | PDLIM1, PHGDH, RAB3D, RPL31, RPL9, |
| | | | | | | | RPLP0, RPS12, RPS23, RPS3, RPS3A, |
| | | | | | | | RPS4X, RPS6, TGM2, UCHL1 |
| IGG | | complex | | 1.765 | | 0.000000149 | AREG, CCR2, CD69, CEBPB, COL3A1, |
| | | | | | | | CXCL10, EMP1, FN1, IL16, IL4, IL5, IL9, |
| | | | | | | | IRF4, LY9, MS4A1, PDPN, SLC16A6, TLR7, |
| | | | | | | | TNFRSF13B, TNFSF15, TSC22D3, TSLP, |
| | | | | | | | VEGFA |
| IL1B | 0.19 | cytokine | | −1.164 | | 0.00000016 | A2M, ACTA2, ATF4, BDNF-AS, CCR2, |
| | | | | | | | CD69, CEBPB, CREM, CXCL10, CXCL13, |
| | | | | | | | CYP2C19, FCER2, FN1, GADD45A, GAS5, |
| | | | | | | | H2BC3, H2BC4, H3C1, H4C15, HK2, IDH1, |
| | | | | | | | IL16, IL9, IRF4, ITGB3, NFKBIZ, NQO1, |
| | | | | | | | NR4A2, NTRK1, PCSK1, PDPN, PHLDA1, |
| | | | | | | | PIGR, PODXL, RPSA, SIPR3, SERPINB9, |
| | | | | | | | SGPL1, SLC14A1, SPP1, TGM2, TINCR, |
| | | | | | | | TLR7, TNFSF15, TPT1, TRAF6, TSC22D3, |
| | | | | | | | TSLP, VEGFA |
| IL5 | −1.157 | cytokine | | −1.165 | | 0.00000016 | A2M, AICDA, AREG, ATF4, BNIP3, CCR2, |
| | | | | | | | CD69, CD79A, CLEC2B, EPS8, GADD45A, |
| | | | | | | | IL13RA1, IL4, IL5, IL5RA, IL9, JCHAIN, |
| | | | | | | | PDLIM1, PSAT1, RBM3, TPM4 |
| IL17A | −0.356 | cytokine | | 0.354 | | 0.00000019 | ACTA2, AREG, CEBPB, COL3A1, CXCL10, |
| | | | | | | | CXCL13, FN1, GADD45A, IL16, IL4, IL5, |
| | | | | | | | IL9, NFKBIZ, NR4A2, PODXL, S1PR3, |
| | | | | | | | SLC12A3, SMAD2, SPP1, TRAF6, TSLP, |
| | | | | | | | VEGFA |
| TP53 | 0.089 | transcription | | 0.829 | | 0.00000026 | A2M, ACTA2, AREG, ATF4, BNIP3, |
| | | regulator | | | | | CCNB1IP1, CCR2, CEBPB, COL3A1, |
| | | | | | | | COL5A2, CXCL10, CYP51A1, DDIT4L, |
| | | | | | | | DMAC2L, DNAJC12, EEF1A1, EPHA2, |
| | | | | | | | EPS8, FABP3, FN1, GADD45A, GAS7, |
| | | | | | | | GDA, GLIPR1, GNA14, H2BC5, H4C15, |
| | | | | | | | HK2, HMGN2, IDH1, IL16, IL4, IL5, IL5RA, |
| | | | | | | | IL9, KLF7, LIAS, MET, MIR-29, MYO1E, |
| | | | | | | | NCAPH, NPTX1, NUCKS1, PADI2, |
| | | | | | | | PDGFRB, PDLIM1, PFKFB4, PHGDH, |
| | | | | | | | PHLDA1, PODXL, RBM3, RPS12, RPS3, |
| | | | | | | | RPS6KA2, RPSA, RRM2, SCD, SCPEP1, |
| | | | | | | | SERPINB9, SFRP1, SGPL1, SLC37A3, |
| | | | | | | | SPP1, TG, TGM2, TLR7, TNFSF15, |
| | | | | | | | TSC22D3, TSPAN6, VEGFA, VLDLR |
| IFNG | −0.559 | cytokine | inhibited | −2.044 | | 0.000000302 | A2M, ACTA2, AREG, ARG2, ASCL1, |
| | | | | | | | ATF4, BDNF-AS, BLNK, BTN3A2, CCR2, |
| | | | | | | | CD2, CEBPB, COL5A2, CREM, CXCL10, |
| | | | | | | | CXCL13, DAXX, EEF1A1, FCER2, FN1, |
| | | | | | | | GAS5, GNA14, GNG7, H2BC3, H2BC8, |
| | | | | | | | HK2, IDH1, IL13RA1, IL4, IL5, IL9, IRF4, |
| | | | | | | | ITGB3, KMO, NFKBIZ, NLRP3, NQO1, |
| | | | | | | | NTRK1, PCSK1, PHGDH, PHLDA1, PIGR, |
| | | | | | | | S1PR3, SELL, SERPINB9, SFRP1, SGPL1, |
| | | | | | | | SLC12A3, SLC14A1, SLC25A5, SPP1, TG, |
| | | | | | | | TINCR, TLR7, TNFSF15, TRAF6, TSC22D3, |
| | | | | | | | VEGFA, VLDLR |
| IMMUNOGLOBULIN | | complex | | 0.465 | | 0.000000425 | ACTA2, AICDA, AREG, BATF3, BPGM, |
| | | | | | | | CCR2, CD300A, CD69, CD8A, CEBPB, |
| | | | | | | | COL3A1, COL5A2, CXCL10, CXCL13, FN1, |
| | | | | | | | GAS7, GPT2, HK2, IL3, IL4, IL5, IL9, IRF4, |
| | | | | | | | JCHAIN, MGST3, MIR-29, MYO1E, |
| | | | | | | | NFKBIZ, NLRP3, RPS3, RPS6, RRM2, SCD, |
| | | | | | | | SELL, SLC41A2, SLC6A9, SPP1, TLR7, |
| | | | | | | | TNFSF15, TRAF6, TSC22D3, TSLP, |
| | | | | | | | VEGFA, VLDLR |
| MIR-155 | | microRNA | | 0.713 | | 0.000000637 | ACTA2, AICDA, ARG2, CD69, CEBPB, |
| | | | | | | | CXCL10, FCER2, HK2, IL13RA1, IL4, IL5, |
| | | | | | | | IRF4, ISYNA1, SMAD2 |
| IL3 | −0.843 | cytokine | | −1.125 | | 0.000000754 | ALDH2, AREG, CD69, CREM, EEF1A1, |
| | | | | | | | FCER2, GADD45A, HK2, HSPA2, IL4, |
| | | | | | | | IL5RA, ITGB3, JCHAIN, LY9, PDPN, |
| | | | | | | | PHGDH, RBM3, RPS12, RPS3, RPS6, SELL, |
| | | | | | | | VEGFA |
| TAFAZZIN | −0.111 | enzyme | | 0 | | 0.000000846 | ACTA2, BANK1, BCL7A, CCR2, CD180, |
| | | | | | | | CD22, CD69, COL5A2, FCER2, FN1, IDH1, |
| | | | | | | | IGKC, IL16, IRF4, MET, MS4A1, SIPR3, |
| | | | | | | | TBC1D2, VEGFA, VLDLR |
| IFN BETA | | group | | −0.892 | | 0.00000126 | AREG, CD22, CD69, CD79A, CEBPB, |
| | | | | | | | CXCL10, DAXX, EEF1A1, FN1, GAS5, IL9, |
| | | | | | | | INSIG1, KLHL14, KMO, SLC16A6, SPP1, |
| | | | | | | | TLR7, TSLP |
| TFRC | −0.051 | transporter | activated | 2.335 | | 0.00000137 | ACTA2, ATF4, BNIP3, CD300A, |
| | | | | | | | GADD45A, GAS7, HK2, PHGDH, PLD4, |
| | | | | | | | SMAD2, STC2 |
| AHR | −0.083 | ligand- | | −0.104 | | 0.00000149 | A2M, ACTA2, ALDH2, AREG, ARG2, |
| | | dependent | | | | | CD8A, COL27A1, COL3A1, COL5A2, |
| | | nuclear | | | | | CXCL10, CXCL13, FN1, GADD45A, HK2, |
| | | receptor | | | | | IL5, IRF4, KDSR, NID1, NLRP3, NPTX1, |
| | | | | | | | NQO1, PDGFRB, SCD, SLAMF6, SLAMF8, |
| | | | | | | | SPP1, VEGFA |
| CREB1 | −0.112 | transcription | | −0.611 | | 0.00000186 | AANAT, BNIP3, CAMK1D, CD79A, |
| | | regulator | | | | | CEBPB, CHRNA1, CIART, CREM, |
| | | | | | | | CYP51A1, DKK2, FN1, GADD45A, GDA, |
| | | | | | | | IL4, ING4, KLF7, NARF, NR4A2, PCSK1, |
| | | | | | | | PDE3A, POLE4, POLR2A, RASGRF1, |
| | | | | | | | S1PR3, SCD, SLC18A2, TG, TPT1, VEGFA |
| CDKN2A | −0.27 | transcription | | 0.421 | | 0.00000191 | A2M, ACTA2, BCL11A, CD79A, CEBPB, |
| | | regulator | | | | | CXCL10, CXCL13, EEF1A1, EPS8, FANCL, |
| | | | | | | | FN1, GAS7, GPT2, IL5RA, ITGB3, MET, |
| | | | | | | | MS4A1, PDGFRB, PHGDH, PSAT1, RRM2, |
| | | | | | | | VEGFA |
| ETS1 | 0.129 | transcription | | −1.485 | | 0.00000206 | BCL11A, CD69, CD79A, FN1, IL3, IL4, IL5, |
| | | regulator | | | | | INSIG1, ITGB3, MET, NQO1, SELL, |
| | | | | | | | SLAMF6, SPP1, TRPC1, ULBP2, VEGFA |
| TCF12 | 0.077 | transcription | | −0.207 | | 0.00000269 | BLNK, CCR2, FN1, IL3, IL4, IL5, IRF4, |
| | | regulator | | | | | JDP2, KLF7, RAG1, RPS3A, ST8SIA1, |
| | | | | | | | VEGFA |
| STAT3 | 0.116 | transcription | | −1.117 | | 0.00000293 | A2M, ACTA2, AICDA, AREG, ARG2, |
| | | regulator | | | | | CEBPB, COL3A1, CXCL10, CXCL13, FN1, |
| | | | | | | | GADD45A, GLIPR1, GRB10, |
| | | | | | | | H2AC18/H2AC19, HK2, IL4, IL5, IL9, IRF4, |
| | | | | | | | LEPR, NFKBIZ, NR4A2, PCSK1, PHLDA1, |
| | | | | | | | POLE4, ROR1, RRM2, SERPINB9, |
| | | | | | | | SIPA1L2, SPP1, STC2, TGM2, TNS4, |
| | | | | | | | VEGFA |
| MSC | 0.103 | transcription | | 0.707 | | 0.00000293 | AICDA, CD79A, FCRL2, IL3, JCHAIN, |
| | | regulator | | | | | SELL, SPP1, STS, TLR7 |
| DDIT4 | −0.248 | other | | −0.816 | | 0.00000333 | AREG, CXCL10, IL4, IL5, NLRP3, SCD, |
| | | | | | | | TSLP |
| IL6 | −1.319 | cytokine | inhibited | −2.441 | | 0.00000345 | A2M, ACTA2, AREG, CCR2, CD79A, |
| | | | | | | | CEBPB, CHAT, COL3A1, CXCL10, |
| | | | | | | | CXCL13, FCER2, FN1, GADD45A, IL4, IL5, |
| | | | | | | | IL9, IRF4, ITGB3, JCHAIN, MET, PCSK1, |
| | | | | | | | PODXL, RAG1, ROR1, RRM2, SIPR3, |
| | | | | | | | SLC14A1, SLC41A2, SPP1, STS, TGM2, |
| | | | | | | | TLR7, TNS4, TRAF6, VEGFA, VLDLR |
| CD40 | −0.283 | transmembr | | −0.284 | | 0.00000393 | AICDA, ATF4, CD22, CD69, CD8A, |
| | | ane receptor | | | | | CXCL10, FCER2, HK2, IL13RA1, IL4, |
| | | | | | | | IL5RA, IRF4, JCHAIN, MET, PPA2, RAG1, |
| | | | | | | | RASGRF1, SELL, TNFRSF13B, VEGFA |
| IL13 | 0 | cytokine | | −1.489 | | 0.00000404 | ARG2, CD1C, CD52, CEBPB, CFI, COL3A1, |
| | | | | | | | CXCL10, FCER2, FCRL2, FCRL3, FN1, |
| | | | | | | | GAS7, HOMER2, IL13RA1, IL4, MAOA, |
| | | | | | | | NCAPH, NID1, PHLDA1, RPS6KA2, |
| | | | | | | | SLC16A6, SPP1, TGM2, TSLP, VEGFA |
| NT5E | 0.513 | phosphatase | | 0.447 | | 0.00000425 | CD69, COL3A1, IL13RA1, IL4, SPP1, |
| | | | | | | | TRAF6 |
| IL2 | −0.397 | cytokine | | −1.669 | | 0.00000512 | AICDA, AREG, CCR2, CD2, CD69, CD8A, |
| | | | | | | | CXCL10, CYP51A1, EMP1, H1-2, HK2, |
| | | | | | | | IDH1, IL3, IL4, IL5, IL9, ING4, INSIG1, |
| | | | | | | | IRF4, MYO1E, PHGDH, PHLDA1, PSAT1, |
| | | | | | | | RPS6, RRM2, SELL, SERPINB9, SIRPG, |
| | | | | | | | SPP1, STC2, TNFRSF13B, TRAF6, VEGFA, |
| | | | | | | | VLDLR |
| PAX5 | −0.607 | transcription | | −1.01 | | 0.00000627 | BLNK, CD22, CD79A, FCER2, FN1, HK2, |
| | | regulator | | | | | IL3, JCHAIN, MET |
| STIM1 | 0.123 | ion channel | inhibited | −3 | | 0.00000627 | ATF4, CAMK1D, KLF7, NQO1, RPS12, |
| | | | | | | | RPS23, SIPR3, TG, VEGFA |
| AREG | 1.429 | growth | inhibited | −2.287 | | 0.00000661 | AREG, H2AC18/H2AC19, H2AC6, H2BC12, |
| | | factor | | | | | H2BC21, H2BC4, H2BC5, H2BC8, IL9, |
| | | | | | | | RRM2 |
| HDL- | | complex | | 1.89 | | 0.00000713 | ALDH1L2, CEBPB, CREM, CYP51A1, |
| CHOLESTEROL | | | | | | | GPT2, NUDT7, VLDLR |
| IL33 | | cytokine | | −1.508 | | 0.00000752 | AREG, BATF3, BNIP3, CCR2, CD69, |
| | | | | | | | COL3A1, CREM, CXCL10, HILPDA, IL4, |
| | | | | | | | IL5, IL9, LEPR, NFKBIZ, NLRP3, PCSK1, |
| | | | | | | | PDLIM1, PSAT1, RPS6KA2, SERPINB9, |
| | | | | | | | TNFRSF13B, TSLP, VEGFA |
| GNAS | 0.075 | enzyme | | 0.898 | | 0.0000088 | BCL7A, BEND5, CD22, CREM, FCER2, |
| | | | | | | | ITGB3, MPEG1, NR4A2, PCSK1, RASD1, |
| | | | | | | | SCD, SIPA1L1, SPP1, TG |
| BIRC5 | −0.028 | other | | | | 0.00000929 | COL3A1, COL5A2, CXCL13, FN1, IL4, IL5, |
| | | | | | | | IL9, JCHAIN |
| NFAT5 | −0.006 | transcription | | −0.466 | | 0.00000943 | AREG, CXCL10, DAXX, FABP3, HSPA2. |
| | | regulator | | | | | IL4, ITGB3, POLE4, RRM2, SEMA4C, |
| | | | | | | | SPP1, VEGFA, VLDLR |
| CREM | 0.351 | transcription | | 0.286 | | 0.0000108 | AANAT, CD8A, CEBPB, CIART, COL3A1, |
| | | regulator | | | | | CREM, CYP51A1, IL4, NR4A2, OAZ3, |
| | | | | | | | PCSK1, PDE3A, VEGFA |
| SYK | −0.298 | kinase | | −1.203 | | 0.0000111 | CD69, CREM, CXCL10, GADD45A, IL3, |
| | | | | | | | IL4, ITGB3, LY9, TSC22D3, VEGFA, |
| | | | | | | | YPEL5 |
| TCR | | complex | inhibited | −2.683 | | 0.0000119 | CCR2, CD69, CXCL10, CXCL13, IL16, IL4, |
| | | | | | | | IL5, IL9, IRF4, RPL31, RPL9, RPLP0, |
| | | | | | | | RPS23, RPS3, RPS3A, RPS4X, RPSA, SELL, |
| | | | | | | | SPP1, TLR7, TSC22D3 |
| STAT6 | −0.088 | transcription | inhibited | −2.16 | | 0.0000133 | ALDH2, AREG, CAMK1D, CD2, CD69, |
| | | regulator | | | | | COL3A1, CXCL10, EPHA2, FCER2, FN1, |
| | | | | | | | GNA14, HNRNPH3, IGF2R, IL4, IL5, |
| | | | | | | | IL5RA, IL9, IRF4, KMO, LACC1, MAOA, |
| | | | | | | | MPEG1, TSLP, TSPAN6 |
| HIF1A | 0.181 | transcription | | −1.763 | | 0.0000135 | ACTA2, BNIP3, CCR2, CD69, CXCL10, |
| | | regulator | | | | | EOLA1/EOLA2, FN1, H1-2, H2AC6, |
| | | | | | | | H2BC12, H4C15, HILPDA, HK2, HMGCL, |
| | | | | | | | IL4, IL9, ITGB3, MET, NFKBIZ, NUCKS1, |
| | | | | | | | PFKFB4, SCD, SFRP1, SMAD2, STC2, |
| | | | | | | | TMEM45A, VEGFA |
| BNIP3L | −0.47 | other | | −1.463 | | 0.0000157 | BLNK, CD22, GADD45A, HK2, IL16, |
| | | | | | | | PDGFRB, RRM2, SFRP1 |
| ELAVL1 | 0.045 | other | | −1.431 | | 0.0000175 | ACTA2, ARG2, ASCL1, CYP51A1, GLIPR1, |
| | | | | | | | HOMER2, HSPA2, IL13RA1, IL4, IRF4, |
| | | | | | | | JCHAIN, MPEG1, PIGR, RPS6, TSC22D3, |
| | | | | | | | VEGFA |
| MYC | −0.584 | transcription | | −1.663 | | 0.0000182 | ACTA2, ASCL1, ATF4, CD69, COL3A1, |
| | | regulator | | | | | COL5A2, CXCL10, DLEU1, EMP1, EPHA2, |
| | | | | | | | FN1, GADD45A, GPT2, H3C1, HK2, |
| | | | | | | | HNRNPA1, IDH1, IL5RA, ING4, IRF4, |
| | | | | | | | JAG2, LHX9, MGST3, MIR-29, NQO1, |
| | | | | | | | PDGFRB, PSAT1, RPL31, RPL9, RPLP0, |
| | | | | | | | RPS12, RPS15A, RPS23, RPS3, RPS6, |
| | | | | | | | RPSA, RRM2, SCD, SCPEP1, SFRP1, |
| | | | | | | | SLC25A5, SNHG26, SPP1, TAF1D, TINCR, |
| | | | | | | | TLR7, VEGFA, VLDLR |
| TCF3 | −0.061 | transcription | | −0.213 | | 0.0000189 | AICDA, AREG, BLNK, CCR2, CD2, |
| | | regulator | | | | | CD79A, GADD45A, IL16, IL4, IL5, IRF4, |
| | | | | | | | JDP2, KLF7, NIBAN3, POLE4, RAG1, |
| | | | | | | | RPS3A, RRM2, SCN4A, SELL, ST8SIA1, |
| | | | | | | | VEGFA |
| SMAD3 | 0.337 | transcription | | −1.454 | | 0.0000198 | ACTA2, AREG, ATF4, CD79A, COL3A1, |
| | | regulator | | | | | CXCL10, CYP51A1, FN1, IL4, IL5, IL9, |
| | | | | | | | MIR-29, POLE4, RPS6, SIPR3, SCD, SPP1, |
| | | | | | | | VEGFA, VLDLR |
| PKC(S) | | group | | 1.03 | | 0.0000203 | ACTA2, ARG2, CCR2, COL3A1, FN1, |
| | | | | | | | GADD45A, MS4A1, NQO1, NR4A2, |
| | | | | | | | PHLDA1, SLC6A9, SPP1, STC2, ULBP2, |
| | | | | | | | VEGFA |
| ID2 | 0.114 | transcription | | 1.609 | | 0.0000264 | ACTA2, AICDA, CCR2, CD79A, CEBPB, |
| | | regulator | | | | | HOMER2, IL4, IRF4, ITGB3, JDP2, KLF7, |
| | | | | | | | LEPR, RPS6KA2, SELL, SLAMF6 |
| MIR-34A-5P | | mature | | 0.22 | bias | 0.0000293 | ACTA2, CD69, CD96, FN1, ITGB3, |
| (AND | | microRNA | | | | | KDM7A, MET, SELL, SHISA2, SLC18A2, |
| OTHER | | | | | | | TSPOAP1, VEGFA |
| MIRNAS | | | | | | | |
| W/SEED | | | | | | | |
| GGCAGUG) | | | | | | | |
| CD3 | | complex | | −1.272 | | 0.0000335 | AARS1, ALKBH1, ATF4, CCR2, CD2, |
| | | | | | | | CD69, CD8A, CREM, CXCL10, |
| | | | | | | | H2AC18/H2AC19, H2AC6, HMGN2, |
| | | | | | | | HNRNPA1, HOMER2, IL16, IL3, IL4, IL5, |
| | | | | | | | IL9, IRF4, ORAI3, POLR2A, RBM3, RPS3A, |
| | | | | | | | SLC25A5, TGM2, TRAF6, VEGFA |
| BRD4 | | kinase | | −0.213 | | 0.0000367 | ACTA2, BLNK, CCR2, COL3A1, COL5A2, |
| | | | | | | | GPR55, HK2, POLE4, SELL, TNFSF15 |
| KLF11 | −0.037 | transcription | inhibited | −2.333 | | 0.0000405 | ACTA2, CCR2, CEBPB, CHRNA1, |
| | | regulator | | | | | COL3A1, FABP3, IL4, IL5, ITGB3, SMAD2, |
| | | | | | | | VEGFA |
| PDGF BB | | complex | | −0.275 | | 0.0000422 | ACTA2, AREG, ARG2, ASCL1, CALD1, |
| | | | | | | | CEBPB, COL3A1, FN1, GADD45A, GDA, |
| | | | | | | | HOMER2, NR4A2, PDGFRB, PDLIM1, |
| | | | | | | | PHLDA1, RPS6, SCD, TGM2, VEGFA |
| RXRB | −0.076 | ligand- | | 1.155 | | 0.0000423 | ATF4, ATPIA3, CAMK1D, CXCL10, H3C1, |
| | | dependent | | | | | IL4, IL5, ITGB3, LHX9, MYO1E, SPP1, |
| | | nuclear | | | | | TSLP, VLDLR |
| | | receptor | | | | | |
| PDGFB | 0.018 | growth | | −1.513 | | 0.0000442 | ACTA2, COL3A1, FN1, MIR-29, PDGFRB, |
| | | factor | | | | | PHLDA1, SPP1, VEGFA |
| MFSD2A | −0.204 | transporter | activated | 2.219 | | 0.0000459 | CYP51A1, HK2, IDH1, INSIG1, SCD |
| ITGA11 | −0.167 | other | | −1 | bias | 0.0000459 | ACTA2, FN1, INSIG1, PDGFRB, SPP1 |
| BHLHE40 | 0.043 | transcription | | −0.158 | | 0.0000499 | AARS1, ACTA2, CD69, GDA, HILPDA, |
| | | regulator | | | | | HK2, IL5, JCHAIN, MET, NLRP3, PCSK1, |
| | | | | | | | PDPN, PHLDA1, PIGR, SELL, SLAMF6, |
| | | | | | | | TGM2, TNFSF15 |
| FOXP3 | −0.21 | transcription | | 1.094 | | 0.000053 | ARHGAP31, CCR2, CD69, CXCL13, IL4, |
| | | regulator | | | | | IL5, IL9, IRF4, SELL, TGM2, TRAF6, |
| | | | | | | | VEGFA, WFDC21P |
| NELFB | 0.032 | other | | −1 | | 0.0000536 | GADD45A, RPLP0, RPS3, VEGFA |
| MED12 | 0.075 | transcription | | | | 0.0000578 | ASCL1, CREB5, POLE4, SLC16A6, SMAD2 |
| | | regulator | | | | | |
| NR3C1 | 0.097 | ligand- | | 0.182 | | 0.0000603 | A2M, ACTA2, CCR2, CD69, CEBPB, |
| | | dependent | | | | | CLCF1, COL3A1, CXCL10, CYP2C19, |
| | | nuclear | | | | | DAXX, EMP1, FN1, GADD45A, GLIPR1, |
| | | receptor | | | | | IDH1, IL5, MAOA, MIR-29, NLRP3, NQO1, |
| | | | | | | | NTRK1, PDPN, PIGR, RABGGTB, RASD1, |
| | | | | | | | SERPINB9, SGPL1, SPP1, TNS4, TSC22D3, |
| | | | | | | | TSLP |
| CSF1 | −0.377 | cytokine | | −1.71 | | 0.0000608 | CCR2, COL23A1, CXCL10, CYP51A1, FN1, |
| | | | | | | | GAS7, GPR68, HK2, IRF4, ITGB3, JDP2, |
| | | | | | | | SFRP1, SPP1, SYNGR1, TPT1, TRAF6, |
| | | | | | | | VEGFA |
| ADAM8 | −0.008 | peptidase | | | | 0.0000637 | CXCL10, IL4, IL5 |
| REL | 0.266 | transcription | | −0.988 | | 0.0000665 | APLF, CCL4L1/CCL4L2, CREM, CXCL10, |
| | | regulator | | | | | FCER2, FN1, GPT2, IL4, IL5, IL9, IRF4, |
| | | | | | | | PHGDH, PIPOX, SCN11A, TNFRSF13B |
| SHC1 | 0.076 | other | | 0.963 | | 0.0000666 | AICDA, CD69, CXCL10, CXCL13, GDA, |
| | | | | | | | IL9, NQO1, PHLDA1 |
| EIF2S1 | 0.079 | translation | | | | 0.0000727 | ADM2, ALDH1L2, ATF4, CEBPB, |
| | | regulator | | | | | GADD45A, PHLDA1, SLC6A9 |
| TLR9 | −0.186 | transmembr | | −0.796 | | 0.0000729 | AICDA, ARG2, CD300A, CD69, CXCL10, |
| | | ane receptor | | | | | FCER2, IL4, IRF4, LIPG, NFKBIZ, |
| | | | | | | | PHLDA1, SGPL1, TLR7, TNFRSF13B, |
| | | | | | | | TSLP |
| MIR-155-5P | | mature | | 1.95 | bias | 0.0000739 | AICDA, CD69, CEBPB, CYP51A1, FCER2, |
| (MIRNAS | | microRNA | | | | | IL13RA1, MET, MYO1E, PDE3A, PODXL, |
| W/SEED | | | | | | | POLE4, SMAD2 |
| UAAUGCU) | | | | | | | |
| TIGIT | 0.2 | other | | −1.982 | | 0.0000763 | IL4, IL5, IL9, IRF4 |
| ZBTB16 | −0.061 | transcription | | −0.683 | | 0.0000804 | CCR2, CD180, CD2, CD8A, CXCL10, GPT2, |
| | | regulator | | | | | IL4, ITGB3, NFKBIZ, SCD, SELL, SLAMF6, |
| | | | | | | | SPP1, TSC22D3 |
| TGFB2 | −1.361 | growth | | −1.591 | | 0.000084 | ACTA2, COL3A1, FN1, LIPG, PDGFRB, |
| | | factor | | | | | SCD, SELL, SMAD2, SPP1, VEGFA |
| PKA | | complex | | −1.077 | | 0.000084 | AREG, CEBPB, CREM, CXCL10, CYP51A1, |
| | | | | | | | IL4, IL5, IL9, NR4A2, PCSK1, TSLP |
| ADRB | | group | | 1.616 | | 0.000084 | AACS, BNIP3, CREM, CXCL10, CYP51A1, |
| | | | | | | | GADD45A, IDH1, INSIG1, NQO1, NR4A2, |
| | | | | | | | SLC6A9 |
| AKT | | group | inhibited | −2.79 | | 0.0000947 | A2M, ACTA2, ATF4, CEBPB, COL3A1, |
| | | | | | | | COQ7, CXCL10, FN1, HGSNAT, HK2, IL4, |
| | | | | | | | IL5, PODXL, RAG1, RASD1, RPL9, RRM2, |
| | | | | | | | SCD, SPP1, VEGFA |
| CASP8 | 0.141 | peptidase | | 1.72 | | 0.000103 | CD69, CXCL10, IL4, IL5, IL9, NLRP3, TSLP |
| MEK | | group | | −1.682 | | 0.000107 | ASCL1, CXCL10, EPHA2, GDA, HGSNAT, |
| | | | | | | | IL4, ITGB3, MIR-29, MS4A1, NQO1, |
| | | | | | | | PDGFRB, PHLDA1, PODXL, RABGGTB, |
| | | | | | | | SEMA4C, SLC16A6, ST8SIA1, TG |
| LAMA4 | −1.478 | enzyme | activated | 2.985 | | 0.000112 | COL3A1, COL5A2, FN1, IDH1, ITGB3, |
| | | | | | | | MET, MGST3, SPP1, VEGFA |
| BRD4 | −0.067 | kinase | inhibited | −2.16 | | 0.000122 | ACTA2, BTN3A2, FN1, GADD45A, IRF4, |
| | | | | | | | NQO1, PDGFRB, RNF125, RRM2, |
| | | | | | | | SLC16A6, SPP1, STC2 |
| CTNNB1 | −0.154 | transcription | inhibited | −2.082 | | 0.000129 | ACTA2, AICDA, ASCL1, COL27A1, |
| | | regulator | | | | | COL3A1, CXCL10, CYP51A1, DKK2, FN1, |
| | | | | | | | FOXB1, HILPDA, HK2, IL4, IL9, IRF4, |
| | | | | | | | JCHAIN, MAOA, MPEG1, NPTX1, NUDT7, |
| | | | | | | | POLR2A, RPL31, RPL9, RPLP0, RPS12, |
| | | | | | | | RPS15A, RPS23, RPS3, RPS6, RPSA, SCD, |
| | | | | | | | SFRP1, SLC16A6, SP5, SPP1, TGM2, |
| | | | | | | | VEGFA |
| EGF | | growth | | −1.744 | | 0.000134 | AREG, CEBPB, COL3A1, CREM, CXCL13, |
| | | factor | | | | | EEF1A1, EMP1, EPHA2, FN1, GADD45A, |
| | | | | | | | H3C1, IDH1, ITGB3, JAG2, MAOA, MET, |
| | | | | | | | NR4A2, PDE3A, PDGFRB, PDPN, PFKFB4, |
| | | | | | | | RRM2, SCD, SPP1, TBX19, TGM2, VEGFA |
| GNAQ | −0.381 | enzyme | | | | 0.000137 | AREG, BNIP3, CLCF1, CREM, EMP1, |
| | | | | | | | GADD45A, HK2, INSIG1, NR4A2, |
| | | | | | | | PHLDA1, VEGFA |
| USP38 | −0.109 | peptidase | | −1.213 | | 0.000141 | ACTA2, FN1, IL4, IL5 |
| NUPR1 | 1.017 | transcription | | 0.396 | | 0.000142 | ACTA2, APOBEC3B, AREG, BNIP3, |
| | | regulator | | | | | CEBPB, COL3A1, CREB5, GADD45A, |
| | | | | | | | H2BC3, H3C1, H3C6, HILPDA, HK2, |
| | | | | | | | HSPA2, IL13RA1, KBTBD7, LINC01963, |
| | | | | | | | NUCKS1, PFKFB4, PHLDA1, SIPA1L2, |
| | | | | | | | SLC16A6, SPP1 |
| TGFB1 | −0.094 | growth | | −1.429 | | 0.000143 | ACTA2, ALDH2, AREG, ATF4, CALD1, |
| | | factor | | | | | CCR2, CD300A, CD69, CDHR1, CEBPB, |
| | | | | | | | CFI, COL3A1, CXCL10, CXCL13, |
| | | | | | | | CYP51A1, DACT1, DAXX, DKK2, EEF1A1, |
| | | | | | | | EPHA2, FAM110B, FCER2, FN1, |
| | | | | | | | GADD45A, GAS7, GNA14, GNG7, HK2, |
| | | | | | | | HMGN2, IDH1, IL4, IL5, IL9, INSIG1, |
| | | | | | | | ITGB3, JAG2, MAOA, MET, MIR-29, |
| | | | | | | | NDST1, NLRP3, NR4A2, PDGFRB, PDPN, |
| | | | | | | | PHGDH, PODXL, PSAT1, RBM3, ROR1, |
| | | | | | | | RRM2, SIPR3, SCD, SFRP1, SMAD2, SPP1, |
| | | | | | | | STC2, TG, TGM2, TRPC1, TSC22D3, |
| | | | | | | | VEGFA, VLDLR |
| ATF4 | −0.52 | transcription | inhibited | −2.62 | | 0.000156 | AARS1, AREG, ATF4, BCL11A, CEBPB, |
| | | regulator | | | | | FN1, GADD45A, GRB10, PHGDH, PSAT1, |
| | | | | | | | SLC6A9, STC2, VEGFA |
| CD28 | 0.407 | transmembr | | −1.369 | | 0.000163 | ALKBHI, CD69, CREM, CXCL13, |
| | | ane receptor | | | | | H2AC18/H2AC19, H2AC6, HK2, HMGN2, |
| | | | | | | | HNRNPA1, HOMER2, IL3, IL4, IL5, IL9, |
| | | | | | | | IRF4, ORAI3, POLR2A, SLC25A5 |
| MAPK3 | −0.166 | kinase | | −0.896 | | 0.000175 | CCR2, CD69, COL3A1, FN1, IL4, IL5, |
| | | | | | | | SELL, SPP1, VEGFA |
| TNFSF15 | 1.42 | cytokine | | −0.425 | | 0.000185 | ACTA2, FN1, IL4, IL5, IL9, SPP1 |
| NREP | −0.21 | other | | 0.218 | | 0.000186 | ACTA2, COL3A1, PDGFRB, VEGFA |
| RAD21L1 | 0.844 | other | | | | 0.000186 | AREG, EPHA2, H1-2, H2BC12, H2BC12L |
| STAG3 | −0.19 | other | | | | 0.000186 | AREG, EPHA2, H1-2, H2BC12, H2BC12L |
| NFKBIA | 0.173 | transcription | | −0.758 | | 0.000189 | A2M, BCL11A, BNIP3, CD69, CEBPB, |
| | | regulator | | | | | COL3A1, COL5A2, CXCL10, FN1, |
| | | | | | | | GADD45A, GAS5, HK2, IGF2R, IL5, JDP2, |
| | | | | | | | NID1, PDLIM1, RPSA, SFRP1, TRPC1, |
| | | | | | | | VEGFA |
| BCL6 | 0.021 | transcription | | 0.949 | | 0.000191 | A2M, AICDA, BLNK, CD2, CD69, CXCL10, |
| | | regulator | | | | | FCER2, GADD45A, IL13RA1, IL4, IL5, IL9, |
| | | | | | | | IRF4, PDIA2 |
| HAVCR1 | −0.121 | other | | 0.104 | | 0.000201 | CEBPB, HK2, IL4, IL5, NUCKS1, STC2, |
| | | | | | | | TMEM45A, UCHL1, VEGFA |
| SPI1 | −0.215 | transcription | | −1.174 | | 0.000215 | ACTA2, BCL11A, BCL7A, BLNK, CD180, |
| | | regulator | | | | | CD79A, CXCL10, GPR84, IL4, IL5, IL9, |
| | | | | | | | IRF4, JDP2, MS4A1, RAG1, SPP1 |
| SMC1B | −0.146 | transporter | | | | 0.00022 | AREG, EPHA2, H1-2, H2BC12, H2BC12L |
| IKBKB | −0.036 | kinase | inhibited | −2.146 | | 0.000223 | ACTA2, CEBPB, COL3A1, CXCL10, |
| | | | | | | | GADD45A, HK2, IL4, IL5, ITGB3, NID1, |
| | | | | | | | NLRP3, RAG1, SFRP1, TRPC1, TSLP, |
| | | | | | | | VEGFA |
| SIRT6 | −0.028 | enzyme | | 0.064 | | 0.00023 | AACS, ACTA2, COL3A1, CYP2C18, FN1, |
| | | | | | | | IGF2R, RPS15A, SCD |
| CCND1 | −0.129 | transcription | | 0.042 | | 0.000245 | AREG, COL27A1, COL5A2, DKK2, EPHA2, |
| | | regulator | | | | | EPS8, H2BC21, HERC3, HOMER2, ITGB3, |
| | | | | | | | LINC00467, MYO7A, NCAPH, PBLD, |
| | | | | | | | RPS6, RRM2, SPP1 |
| YAP1 | −0.546 | transcription | | 0.591 | | 0.000251 | ACTA2, AREG, C10ORF143, FN1, |
| | | regulator | | | | | GADD45A, JAG2, LINC00467, MIR-29, |
| | | | | | | | RPL31, RPL9, RPLP0, RPS12, RPS15A, |
| | | | | | | | RPS23, RPS3, RPS6, RPSA, RRM2, S1PR3, |
| | | | | | | | SMAD2, TBC1D2, TRAF6, VEGFA |
| IGF1 | −0.149 | growth | | −1.148 | | 0.000252 | A2M, ACTA2, APLF, CEBPB, CHAT, |
| | | factor | | | | | COL3A1, COL5A2, DAXX, EIF4B, FJX1, |
| | | | | | | | FN1, GADD45A, IL16, IL4, JDP2, LEPR, |
| | | | | | | | NTRK1, PDE3A, PDGFRB, PHLDA1, SCD, |
| | | | | | | | SPP1, TG, TGM2, VEGFA |
| EGLN | | group | | 1.793 | bias | 0.000253 | A2M, ADM2, ASCL1, BNIP3, H3C1, H4C15, |
| | | | | | | | HK2, HMGCL, STC2, VEGFA, VLDLR |
| TLR7 | −1.275 | trans- | inhibited | −2 | bias | 0.00026 | AICDA, BANK1, CD300A, CD69, CD79A, |
| | | membrane | | | | | CEBPB, CREB5, CXCL10, CXCL13, GPR84, |
| | | receptor | | | | | LHFPL1, SLC12A3, TLR7 |
| BCAP31 | 0.118 | transporter | | −0.968 | | 0.000265 | CEBPB, CXCL10, FN1, INSIG1, SCD, |
| | | | | | | | VEGFA |
| FYN | 0.259 | kinase | | −1.446 | | 0.000265 | CXCL10, IL4, IL5, IL5RA, IRF4, NLRP3 |
| ADRA1B | −0.268 | g-protein | | | | 0.000265 | CEBPB, COL3A1, CREM, FN1, NR4A2, |
| | | coupled | | | | | VEGFA |
| | | receptor | | | | | |
| ADRA1D | | g-protein | | | | 0.000265 | CEBPB, COL3A1, CREM, FN1, NR4A2, |
| | | coupled | | | | | VEGFA |
| | | receptor | | | | | |
| EBF1 | −0.235 | transcription | inhibited | −2.182 | | 0.000267 | CCL4L1/CCL4L2, CD300A, CD79A, |
| | | regulator | | | | | CEBPB, CXCL10, FCER2, HK2, IGKC, IL3, |
| | | | | | | | RAG1, SCD |
| MED1 | −0.112 | transcription | | −1.671 | | 0.000271 | BCL11A, BCL7A, CD79A, CREB5, |
| | | regulator | | | | | GADD45A, HILPDA, IRF4, MET, PODXL, |
| | | | | | | | POLE4, SCD, SCPEP1, STC2 |
| IL10 | −0.008 | cytokine | | 1.633 | | 0.000274 | ACTA2, AICDA, ARG2, CCR2, CD2, |
| | | | | | | | CXCL10, CXCL13, FCER2, IL13RA1, IL3, |
| | | | | | | | IL4, IL5, IL9, PHGDH, SELL, SPP1, TLR7, |
| | | | | | | | TNFRSF13B, TRAF6, TSC22D3, VEGFA |
| KDM1A | 0.09 | enzyme | | 0.351 | | 0.000296 | A2M, ACTA2, BCL7A, CD22, CEBPB, |
| | | | | | | | CXCL10, CXCL13, FN1, LY9, MPEG1, |
| | | | | | | | PDE3A, PDPN, POLE4, PSAT1, RAG1, |
| | | | | | | | SCD, SFRP1, SNHG7 |
| PAF1 | 0.135 | other | | 0.762 | bias | 0.000297 | CCL4L1/CCL4L2, H2AC6, H2BC5, HCG4, |
| | | | | | | | NFKBIZ, ULBP2 |
| SREBF1 | −0.191 | transcription | inhibited | −3.191 | | 0.000299 | AACS, AARS1, CAMK1D, CEBPB, CF1, |
| | | regulator | | | | | CXCL10, CYP51A1, FABP3, FN1, HK2, |
| | | | | | | | IDH1, INSIG1, RPLP0, SCD, VEGFA |
| MIRLET7 | | group | | 0 | | 0.000303 | ACTA2, BCL11A, COL5A2, RRM2 |
| ARNT | −0.009 | transcription | | −1.795 | | 0.000329 | AREG, BNIP3, CEBPB, CXCL10, HK2, |
| | | regulator | | | | | ITGB3, NQO1, SELL, SPP1, TSC22D3, |
| | | | | | | | VEGFA |
| CHIT1 | | enzyme | | | | 0.000342 | ACTA2, FN1, SMAD2 |
| IL-2R | | complex | | | | 0.000342 | IL4, IL5, PHGDH |
| GUCY | | group | | | | 0.000351 | IL4, IL5 |
| A2M-AS1 | | other | | | | 0.000351 | CD2, SELL |
| GAS5-AS1 | −0.022 | other | | | | 0.000351 | ACTA2, SMAD2 |
| ITGB2 | 0.114 | trans- | | −0.128 | | 0.000371 | ACTA2, CD69, CXCL10, FN1, IL4, VEGFA |
| | | membrane | | | | | |
| | | receptor | | | | | |
| HNF1A-AS1 | | other | inhibited | −2 | | 0.000378 | H2AC6, H2BC12, H2BC21, H2BC5 |
| DUSP10 | 0.211 | phosphatase | | 0.392 | | 0.000378 | ACTA2, COL3A1, FN1, IL4 |
| SMAD2 | −0.395 | transcription | | −1.681 | | 0.000391 | ACTA2, COL3A1, CYP51A1, FN1, IL4, IL9, |
| | | regulator | | | | | MET, SMAD2, VEGFA |
| NFATC1 | 0.232 | transcription | | −1.4 | | 0.000391 | FCER2, IL4, IL5, ITGB3, NQO1, RAG1, |
| | | regulator | | | | | SELL, SLAMF6, SPP1 |
| BMP10 | −1.585 | growth | | −0.707 | | 0.000392 | A2M, CFI, COL3A1, CXCL13, FN1, LEPR, |
| | | factor | | | | | NR4A2, PHLDA1 |
| SNCA | 1.134 | enzyme | | −0.246 | | 0.000399 | CD22, CDHR1, DGKG, DNAJC12, EMP1, |
| | | | | | | | EPS8, GADD45A, GDA, HOMER2, |
| | | | | | | | IL13RA1, IL4, NLRP3, NPTX1, NQO1, |
| | | | | | | | NR4A2, PSD, SLC18A2, TSC22D3, UCHL1 |
| TREM1 | | trans- | | −0.093 | | 0.000401 | AREG, CEBPB, CXCL10, FABP3, IL4, IL5, |
| | | membrane | | | | | LY9, NR4A2, PHLDA1, SIPR3, SPP1, |
| | | receptor | | | | | TNFSF15 |
| ID3 | | transcription | | 1.671 | | 0.000401 | CD79A, CEBPB, HOMER2, IL4, IRF4, |
| | | regulator | | | | | ITGB3, JDP2, KLF7, LEPR, RPS6KA2, |
| | | | | | | | SELL, SLAMF6 |
| IL18 | 0.03 | cytokine | | −0.28 | | 0.000401 | CCR2, CD69, CXCL10, IL4, IL5, IL9, |
| | | | | | | | NFKBIZ, SELL, SMAD2, SPP1, ULBP2, |
| | | | | | | | VEGFA |
| GNA14 | −2.841 | enzyme | | | | 0.000402 | BNIP3, CREM, GADD45A, HK2, NR4A2, |
| | | | | | | | PHLDA1, VEGFA |
| TEAD3 | −0.396 | transcription | | −1.633 | | 0.000413 | ACTA2, CALD1, COL3A1, COL5A2, FJX1, |
| | | regulator | | | | | NFKBIZ |
| ADRA1A | | g-protein | | | | 0.000413 | CEBPB, COL3A1, CREM, FN1, NR4A2, |
| | | coupled | | | | | VEGFA |
| | | receptor | | | | | |
| BTK | −0.323 | kinase | | −1.091 | | 0.000416 | AICDA, CD2, CD69, CEBPB, CXCL10, |
| | | | | | | | EMP1, IL5, IRF4, ITGB3 |
| TGFB3 | 0.178 | growth | | −0.894 | | 0.000416 | ACTA2, COL3A1, FN1, ITGB3, MS4A1, |
| | | factor | | | | | S1PR3, SCD, SMAD2, VEGFA |
| IL1 | | group | | −1.785 | | 0.000417 | AREG, CXCL10, FN1, IL3, IL4, IL5, IL5RA, |
| | | | | | | | IL9, IRF4, NTRK1, PDGFRB, PIGR, SELL, |
| | | | | | | | TG, TGM2, TLR7, VEGFA |
| NR4A2 | 0.982 | ligand- | | 0.898 | | 0.000421 | AREG, IL4, IL5, MET, NR4A2, SLC18A2, |
| | | dependent | | | | | SPP1, TSC22D3 |
| | | nuclear | | | | | |
| | | receptor | | | | | |
| SREBF2 | −0.048 | transcription | | −1.433 | | 0.000421 | AACS, AREG, CAMK1D, CYP51A1, IDH1, |
| | | regulator | | | | | INSIG1, NLRP3, SCD |
| ATF3 | −0.209 | transcription | | −1.227 | | 0.000423 | ATF4, CCL4L1/CCL4L2, CD69, IL4, |
| | | regulator | | | | | NFKBIZ, PHGDH, POLR2A, PSAT1, RRM2, |
| | | | | | | | TSLP |
| KCNIP3 | | transcription | | 0.378 | | 0.000437 | AANAT, CCR2, CREM, IL16, IL4, SLAMF8, |
| | | regulator | | | | | TGM2 |
| TERT | −0.599 | enzyme | | −1.412 | | 0.000439 | BNIP3, CALD1, COL5A2, CXCL10, |
| | | | | | | | CXCL13, IL13RA1, IL4, IL5, LIPG, |
| | | | | | | | NUCKS1, RRM2, SPP1 |
| FMR1 | 0.099 | translation | | 1.976 | | 0.000453 | CNTNAP4, COL5A2, NPTX1, RASGRF1, |
| | | regulator | | | | | RPL31, RPL9, RPLP0, RPS12, RPS15A, |
| | | | | | | | RPS23, RPS6, RPSA, SYNGR1 |
| ABCG1 | −0.301 | transporter | | 1.387 | | 0.000458 | CXCL13, IL13RA1, IL4, IL5, IL5RA, |
| | | | | | | | INSIG1 |
| FHL2 | 0.639 | transcription | | −0.64 | | 0.000458 | ACTA2, CXCL10, IL4, IL5, PDGFRB, SPP1 |
| | | regulator | | | | | |
| ALDH2 | −0.892 | enzyme | | 1.432 | | 0.000463 | ACTA2, ATF4, FN1, PHGDH, PSAT1 |
| PRKCZ | 0.36 | kinase | | −0.167 | | 0.000463 | IL4, IL5, PHGDH, PSAT1, VEGFA |
| AGPAT2 | 0.051 | enzyme | | 1 | | 0.000466 | CEBPB, DGKG, PLD4, SCD |
| SUCNR1 | −1.022 | g-protein | | 0 | | 0.000466 | ACTA2, COL3A1, COL5A2, NID1 |
| | | coupled | | | | | |
| | | receptor | | | | | |
| PDCD1 | 0.967 | trans- | | 0.055 | | 0.000475 | CD8A, FCRL3, IL4, IL5, NLRP3, PADI2, |
| | | membrane | | | | | SH2D1B |
| | | receptor | | | | | |
| INSULIN | | group | | −1.452 | | 0.000475 | ARG2, BPGM, CEBPB, CHAT, COL3A1, |
| | | | | | | | FABP3, FN1, GRB10, HAP1, HK2, IDH1, |
| | | | | | | | IL4, INSIG1, ITGB3, LEPR, MAOA, MIR- |
| | | | | | | | 29, NLRP3, NR4A2, PDGFRB, PDIA2, |
| | | | | | | | RPS6, RRM2, SCD, SLC25A5, VEGFA, |
| | | | | | | | VLDLR |
| CG | | complex | | −0.872 | | 0.000484 | ACTA2, AREG, ATF4, C3ORF80, CEBPB, |
| | | | | | | | CREB5, CREM, CXCL10, CYP51A1, EMP1, |
| | | | | | | | FJX1, HILPDA, HK2, ITGB3, LEPR, |
| | | | | | | | PDLIM1, PHLDA1, PODXL, RASD1, |
| | | | | | | | SMAD2, SPP1, ST8SIA1, VEGFA |
| MLXIPL | | transcription | inhibited | −3.101 | bias | 0.000498 | RPL31, RPL9, RPLP0, RPS12, RPS15A, |
| | | regulator | | | | | RPS23, RPS3, RPS6, RPSA, SCD |
| ARHGAP5 | 0.025 | enzyme | | | | 0.000506 | ACTA2, COL3A1, FN1 |
| CD48 | 0.067 | other | | | | 0.000506 | IL4, IL5, SERPINB9 |
| EIF2AK4 | −0.2 | kinase | inhibited | −2.395 | | 0.000508 | ATF4, CYP2C18, NQO1, PHGDH, PSAT1, |
| | | | | | | | SCD |
| SORL1 | 0.047 | transporter | inhibited | −2.714 | | 0.000513 | COL3A1, COL5A2, FN1, IL16, MET, NID1, |
| | | | | | | | PDGFRB, ROR1, SFRP1, TGM2, VEGFA |
| IL4R | 0.093 | trans- | | −0.187 | | 0.000519 | AICDA, CD69, CEBPB, FCER2, IL4, IL5, |
| | | membrane | | | | | IL9, JCHAIN |
| | | receptor | | | | | |
| MIR-450A-5P | | mature | activated | 2.236 | bias | 0.00053 | COL3A1, COL5A2, PDGFRB, SMAD2, |
| (AND | | microRNA | | | | | SPP1 |
| OTHER | | | | | | | |
| MIRNAS | | | | | | | |
| W/SEED | | | | | | | |
| UUUGCGA) | | | | | | | |
| NFIL3 | −0.13 | transcription | inhibited | −2.2 | | 0.00053 | BATF3, GADD45A, IL3, IL9, SCD |
| | | regulator | | | | | |
| POU2F1 | −0.356 | transcription | | | | 0.00053 | A2M, CD79A, GADD45A, GAS5, H2BC21, |
| | | regulator | | | | | IDH1, IGKC, IL5, SPP1 |
| BMP7 | −0.942 | growth | | −1.089 | | 0.000532 | ACTA2, CALD1, CCR2, CEBPB, COL5A2, |
| | | factor | | | | | FN1, GRB10, MAOA, PDGFRB, SMAD2, |
| | | | | | | | SPP1, STS, VEGFA |
| PRDM1 | −0.006 | transcription | | 0.122 | | 0.000534 | AICDA, ARG2, BCL11A, CD22, CD79A, |
| | | regulator | | | | | CLEC2B, FCER2, GPR84, |
| | | | | | | | H2AC18/H2AC19, IRF4, JCHAIN, NLRP3, |
| | | | | | | | SCARA5, SELL, TNFRSF13B |
| IL21 | −0.452 | cytokine | | 0.183 | | 0.000538 | AICDA, ARG2, CD2, CD69, CXCL10, |
| | | | | | | | CXCL13, IL4, IL5, IL9, IRF4, SELL |
| SATB2 | −0.715 | transcription | | −0.152 | | 0.000561 | CD96, CHAT, CXCL10, IL3, PDE3A, SPP1 |
| | | regulator | | | | | |
| MTORC1 | | complex | inhibited | −2.087 | | 0.000561 | ATF4, HK2, IL4, INSIG1, SCD, UCHL1 |
| EDN1 | −0.099 | cytokine | | −1.02 | | 0.000571 | ACTA2, AREG, FABP3, FN1, ITGB3, LEPR, |
| | | | | | | | RPSA, SELL, TGM2, TPM4, TPT1, VEGFA |
| CALCA | −1.865 | other | | 0.887 | | 0.000591 | COL3A1, CREM, EIF4B, HILPDA, IL5, IL9, |
| | | | | | | | P2RX3, PCSK1, PDLIM1, PSAT1, |
| | | | | | | | SERPINB9 |
| IKZF2 | −0.318 | transcription | | 0.447 | bias | 0.000603 | CD2, CD69, IL5, IRF4, KLF7 |
| | | regulator | | | | | |
| MYB | −0.03 | transcription | | −1.742 | | 0.000619 | BANK1, BLNK, CARNS1, DHRS2, FN1, |
| | | regulator | | | | | HERC3, IL4, IL5, METTL7B, RAG1, SPP1 |
| NFE2L2 | 0.172 | transcription | | −1.966 | | 0.000648 | ATF4, BNIP3, COL3A1, COQ7, CXCL10, |
| | | regulator | | | | | FN1, GNA14, IDH1, IGF2R, IL4, IL5, |
| | | | | | | | LARP7, MGST3, NDST1, NQO1, PHGDH, |
| | | | | | | | PSAT1, RPLP0, SCD, SLC6A9, UCHL1, |
| | | | | | | | VEGFA |
| CD44 | 0.428 | other | | −1.835 | | 0.000648 | ACTA2, CCR2, CD69, CD8A, COL3A1, |
| | | | | | | | COL5A2, FN1, GADD45A, MET, PFKFB4, |
| | | | | | | | SELL, SPP1, TRAF6 |
| IL15 | 0.191 | cytokine | | −1.117 | | 0.000649 | ATF4, BPGM, CCR2, CD2, CD69, CD8A, |
| | | | | | | | CEBPB, CXCL10, H2AC18/H2AC19, HK2, |
| | | | | | | | IL4, IL5, IL9, RPS3A, RPS6, SELL, |
| | | | | | | | SLC25A5, SMAD2, ULBP2, VEGFA |
| IGF1R | −0.155 | trans- | | −0.652 | | 0.000657 | ACTA2, CD300A, CEBPB, COL3A1, |
| | | membrane | | | | | DMAC2L, EMP1, GRB10, IGF2R, IL16, |
| | | receptor | | | | | RBM3, RPL9, RRM2, SCARA5, SPP1, |
| | | | | | | | VEGFA |
| CD40LG | 0.131 | cytokine | inhibited | −2.045 | | 0.000669 | AICDA, BATF3, BNIP3, CCR2, CD1C, |
| | | | | | | | CD69, CD8A, CXCL10, FCER2, GADD45A, |
| | | | | | | | HK2, IL13RA1, IL4, IRF4, SELL, TGM2, |
| | | | | | | | TNFRSF13B, VEGFA |
| MIR-29 | −3.808 | microRNA | | 1.128 | | 0.000678 | ACTA2, COL3A1, COL5A2, FN1, MIR-29, |
| | | | | | | | NID1, PDGFRB, VEGFA |
| CD5 | 0.33 | trans- | | −1.732 | | 0.00068 | CD52, CD69, CEBPB, IL13RA1, IL5, IL5RA |
| | | membrane | | | | | |
| | | receptor | | | | | |
| APP | 0.132 | other | inhibited | −2.018 | | 0.000699 | ACTA2, ASCL1, ATF4, ATPIA3, CD69, |
| | | | | | | | CEBPB, CHAT, CXCL10, DAXX, FABP3, |
| | | | | | | | FN1, H2BC5, HAP1, HNRNPA1, IGF2R, |
| | | | | | | | IL4, IL5, IL9, IRF4, ITGB3, NLRP3, NPTX1, |
| | | | | | | | NR4A2, PDGFRB, RBM3, RPS6, SLAMF8, |
| | | | | | | | ST8SIA1, TLR7, TNFSF15, TPT1, TRAF6, |
| | | | | | | | UCHL1, VEGFA |
| PTEN | 1.201 | phosphatase | | 0.449 | | 0.000706 | BPGM, CEBPB, COL27A1, COL3A1, |
| | | | | | | | COL5A2, CXCL10, CXCL13, FN1, GRB10, |
| | | | | | | | ING4, ITGB3, JAG2, MET, NQO1, NR4A2, |
| | | | | | | | NTRK1, PDGFRB, PHGDH, RPS6, SCD, |
| | | | | | | | SELL, SFRP1, SMAD2, SPP1, TG, |
| | | | | | | | TSC22D3, TSPO2, VEGFA |
| IL9R | | trans- | | | | 0.000713 | AREG, IL5, SELL |
| | | membrane | | | | | |
| | | receptor | | | | | |
| MYDGF | −0.06 | cytokine | | | | 0.000713 | IL4, IL5, IL9 |
| SMAD7 | 0.057 | transcription | | −0.135 | | 0.000714 | ACTA2, AREG, COL3A1, CXCL10, FN1, |
| | | regulator | | | | | GADD45A, MET, MIR-29, SCD, VEGFA |
| LTA | 0.37 | cytokine | | −1.763 | | 0.000747 | CXCL13, IL4, IL5, ITGB3, PDPN, SELL |
| PTPRJ | 0.044 | phosphatase | | 0 | | 0.000747 | ACTA2, CD69, MET, NFKBIZ, PADI2, TG |
| NRG1 | | growth | | −1.949 | | 0.00075 | ASCL1, ATF4, BNIP3, EEF1A1, EPHA2, |
| | | factor | | | | | FN1, GDA, HK2, ITGB3, NTRK1, PDE3A, |
| | | | | | | | PHGDH, TGM2, VEGFA |
| TRAF3 | 0.022 | enzyme | | −0.656 | | 0.000771 | AICDA, CXCL10, CXCL13, FCER2, IL4, |
| | | | | | | | LACC1, MET, MPEG1 |
| TGFBR2 | 0.18 | kinase | | 0.658 | | 0.000771 | ACTA2, AREG, ATF4, BFSP1, CD69, |
| | | | | | | | CDHR1, DNAJC12, FN1, H2BC5, HILPDA, |
| | | | | | | | HK2, IL4, IL5, NFKBIZ, PDPN, SPP1, STC2, |
| | | | | | | | VEGFA |
| LEF1 | 0.105 | transcription | | 0.367 | | 0.000776 | ACTA2, CD79A, FN1, GPR15, IL4, IL5, |
| | | regulator | | | | | KIF13A, NLRP3, PHLDA1, SLC41A2, SP5, |
| | | | | | | | SPP1, TBC1D2, TLR7, TMEM221 |
| IL12 | | group | | 1.39 | | 0.000788 | CD69, CD8A, IL4, IL5, IL9, IRF4, NFKBIZ, |
| (FAMILY) | | | | | | | SELL, SH2D1B, ULBP2 |
| CCK | | other | | 0.437 | | 0.000815 | CREM, CXCL10, MET, VEGFA |
| LTB | | cytokine | | −1.246 | | 0.000815 | CXCL13, IL4, IL5, SELL |
| MBP | 0.105 | other | | 0.577 | | 0.000815 | CD300A, IL4, IL5, LEPR |
| LTBR | 0.08 | trans- | | −1.664 | | 0.000819 | ACTA2, BATF3, CXCL13, GDA, PDGFRB, |
| | | membrane | | | | | PDPN |
| | | receptor | | | | | |
| CCL2 | | cytokine | | −1.368 | | 0.000819 | CCR2, CXCL10, IL4, IL5, ITGB3, SPP1 |
| CD274 | −0.506 | enzyme | | 0.478 | | 0.000821 | ACTA2, ATF4, CD69, FANCL, IL4, |
| | | | | | | | RPS6KA2, VEGFA |
| NFAT | | group | inhibited | −2.176 | | 0.000821 | CALDI, FCER2, IL3, IL4, IL5, IRF4, |
| (FAMILY) | | | | | | | KIF13A, SMAD2 |
| MIR-17-5P | | mature | | 1.449 | bias | 0.000821 | ACTA2, BLNK, MYLIP, RRM2, SLC16A6, |
| (AND | | microRNA | | | | | TBC1D2, TLR7, VEGFA |
| OTHER | | | | | | | |
| MIRNAS | | | | | | | |
| W/SEED | | | | | | | |
| AAAGUGC) | | | | | | | |
| BCR | | complex | | −1.126 | | 0.000828 | AICDA, BANK1, CD1C, CD22, CD300A, |
| (COMPLEX) | | | | | | | CD69, CLCF1, FCER2, HK2, IL4, IRF4, |
| | | | | | | | PPA2, SELL, TLR7, TNFRSF13B |
| ERK1/2 | | group | | −0.915 | | 0.000884 | AREG, ATP1A3, CEBPB, COL3A1, |
| | | | | | | | CXCL10, FN1, HK2, IL4, ITGB3, LEPR, |
| | | | | | | | PFKFB4, SMAD2, SPP1, TSLP, VEGFA |
| KRAS | 0.11 | enzyme | | −0.772 | | 0.000914 | AREG, ARG2, BNIP3, CEBPB, COL3A1, |
| | | | | | | | CXCL10, EEF1A1, EPHA2, FN1, |
| | | | | | | | GADD45A, GPR68, H1-2, H2AC6, H2BC4, |
| | | | | | | | H3C1, HK2, ITGB3, LIPG, MET, MIR-29, |
| | | | | | | | NLRP3, NQO1, PDIA2, PDPN, PODXL, |
| | | | | | | | RPS6, RPSA, SCD, SLAMF8, SPP1, TGM2, |
| | | | | | | | VEGFA |
| RAS | | group | inhibited | −2.375 | | 0.00093 | ARG2, BNIP3, CXCL10, FN1, IL4, MET, |
| | | | | | | | TG, TGM2, VEGFA |
| ERK | | group | inhibited | −2.029 | | 0.000944 | ACTA2, AREG, ARG2, COL3A1, CXCL10, |
| | | | | | | | EPHA2, FN1, ITGB3, MS4A1, NFKBIZ, |
| | | | | | | | SELL, TGM2, VEGFA |
| IFN | | group | | −1.195 | | 0.000949 | CCR2, CD69, CXCL10, DAXX, GAS7, |
| ALPHA/BETA | | | | | | | SERPINB9, TLR7 |
| TPH1 | 0.019 | enzyme | | −1.89 | bias | 0.000949 | KMO, RPS15A, RRM2, SH2D1B, SLAMF6, |
| | | | | | | | SLC16A6, TG |
| WNT5B | −0.491 | other | | | | 0.000966 | ACTA2, ATF4, FN1 |
| NPAT | −0.021 | transcription | | | | 0.000966 | H2AC6, H2BC21, H2BC5 |
| | | regulator | | | | | |
| CSF | | group | | −0.816 | bias | 0.000978 | CARD16, CD22, FCER2, GPR84, |
| | | | | | | | SERPINB9, TGM2 |
| ERBB2 | −0.057 | kinase | inhibited | −2.46 | | 0.000981 | ACTA2, AREG, ATF4, BNIP3, CEBPB, |
| | | | | | | | COL3A1, COL5A2, CXCL10, CXCL13, |
| | | | | | | | CYP51A1, EMP1, FN1, HGSNAT, |
| | | | | | | | HOMER2, IDH1, IGF2R, ITGB3, JAG2, |
| | | | | | | | JCHAIN, MAOA, MIR-29, NDST1, NID1, |
| | | | | | | | PHGDH, PODXL, POLE4, POLR2A, RRM2, |
| | | | | | | | SFRP1, SMAD2, SP5, TNS4, VEGFA |
| P38 MAPK | | group | inhibited | −2.405 | | 0.001 | ARG2, BNIP3, CD69, CEBPB, CHAT, |
| | | | | | | | COL3A1, CXCL10, FCER2, FN1, IL5, |
| | | | | | | | ITGB3, SMAD2, SPP1, TSLP, ULBP2, |
| | | | | | | | VEGFA, VLDLR |
| RXRA | −0.75 | ligand- | | 1.069 | | 0.00102 | ARG2, ATF4, CAMKID, CXCL10, FABP3, |
| | | dependent | | | | | H3C1, IL4, IL5, INSIG1, ITGB3, LHX9, |
| | | nuclear | | | | | MYO1E, NTRK1, SCD, SPP1, TSLP, |
| | | receptor | | | | | VEGFA, VLDLR |
| STARD7 | −0.076 | other | | | | 0.00104 | IL4, IL5 |
| ZFP35 | | other | | | | 0.00104 | IL4, IL5 |
| EPX | −0.176 | enzyme | | | | 0.00104 | IL4, IL5 |
| HSPG | | group | | | | 0.00104 | AICDA, VEGFA |
| DSG1 | | other | | | | 0.00104 | IL5, TSLP |
| IL27RA | | trans- | activated | 2.207 | | 0.00109 | CD69, CXCL10, CXCL13, IL4, IL5 |
| | | membrane | | | | | |
| | | receptor | | | | | |
| C3 | 0.511 | peptidase | | −0.378 | | 0.00109 | ACTA2, CXCL10, FN1, IL4, PDIA2, SELL, |
| | | | | | | | VEGFA |
| MAPK7 | −0.017 | kinase | | −0.64 | | 0.00109 | AACS, CXCL10, CYP51A1, INSIG1, NQO1, |
| | | | | | | | PDE3A, VEGFA |
| IRF4 | −0.587 | transcription | | 0.415 | | 0.0011 | AICDA, ATF4, BNIP3, CXCL10, FCER2, |
| | | regulator | | | | | IL4, IL9, IRF4, MS4A1, RPS3A, SPP1 |
| VEGF | | group | | −0.81 | | 0.00113 | A2M, ACTA2, CLCF1, CXCL10, EMP1, |
| | | | | | | | FN1, GRB10, GTDC1, HK2, IL4, IL5RA, |
| | | | | | | | INSIG1, IRF4, MET, NR4A2, PHLDA1, |
| | | | | | | | RNF125, SFRP1, SHISA2, TNFSF15, |
| | | | | | | | VEGFA |
| COMMD1 | 0.174 | transporter | activated | 2 | bias | 0.00113 | BNIP3, HK2, STC2, VEGFA |
| RRP1B | −0.087 | transcription | | | | 0.00118 | AREG, ASCL1, CEBPB, DNAJC12, EIF4B, |
| | | regulator | | | | | GADD45A, RPS3, SPP1 |
| INSIG1 | −0.836 | other | | 1.495 | | 0.00118 | AACS, ATF4, CXCL10, CXCL13, CYP51A1, |
| | | | | | | | LIPG, NFKBIZ, SCD |
| WNT1 | | cytokine | | −1.387 | | 0.00121 | ACTA2, COL3A1, DDIT4L, DKK2, |
| | | | | | | | DNAJC1, EEF1A1, FN1, RPS6, SPP1 |
| MHC II | | group | | | | 0.00121 | EEF1A1, IGKC, JCHAIN, RPLP0, TPT1 |
| CD2 | 0.318 | trans- | | | | 0.00121 | CD8A, IL4, IL5, RAG1, SELL |
| | | membrane | | | | | |
| | | receptor | | | | | |
| PDX1 | | transcription | | 0.24 | | 0.00124 | ATF4, COL3A1, CREM, FBXL17, HAP1, |
| | | regulator | | | | | IDH1, INSIG1, NQO1, PCSK1, PHGDH, |
| | | | | | | | SPP1 |
| ADORA2A | 0.629 | g-protein | | −0.245 | | 0.00127 | ATP1A3, COL3A1, CXCL10, GNG7, IL4, |
| | | coupled | | | | | NR4A2, PCSK1, TSC22D3, VEGFA |
| | | receptor | | | | | |
| CCR3 | −0.728 | g-protein | | | | 0.00127 | CCR2, IL4, SPP1 |
| | | coupled | | | | | |
| | | receptor | | | | | |
| RAMP1 | −0.296 | g-protein | | | | 0.00127 | IL4, IL5, VEGFA |
| | | coupled | | | | | |
| | | receptor | | | | | |
| TNFRSF4 | 0.263 | trans- | | −1.098 | | 0.00132 | CXCL10, IL4, IL5, IL9 |
| | | membrane | | | | | |
| | | receptor | | | | | |
| MIR-140 | | microRNA | | | | 0.00132 | ACTA2, BNIP3, COL3A1, VEGFA |
| CD38 | −0.087 | enzyme | | −1.353 | | 0.00132 | ACTA2, AICDA, BNIP3, EPS8, FN1, IL5RA, |
| | | | | | | | JCHAIN, PDLIM1, PSAT1, SELL |
| POSTN | | other | | −1 | | 0.00135 | ACTA2, ATF4, FN1, SPP1, TSLP |
| TLR2 | 0.013 | trans- | | −1.417 | | 0.00135 | ACTA2, CCR2, CD1C, CD69, CEBPB, |
| | | membrane | | | | | CXCL10, FN1, GADD45A, IL4, IL5, SCD |
| | | receptor | | | | | |
| BCL3 | 0.097 | transcription | | −1.353 | | 0.00137 | CD69, CXCL10, FABP3, IL4, IL5, VLDLR |
| | | regulator | | | | | |
| LGALS3 | −0.014 | other | | −0.747 | | 0.0014 | CCR2, CEBPB, CLEC2B, COL3A1, |
| | | | | | | | COL5A2, FN1, IL5, SPP1 |
| CCR2 | −1.545 | g-protein | | 0.314 | | 0.0014 | CCR2, CXCL10, CXCL13, FN1, IL4, IL5, |
| | | coupled | | | | | TGM2, TSLP |
| | | receptor | | | | | |
| IKBKG | 0.136 | kinase | | −0.788 | | 0.0014 | CEBPB, CXCL10, FCER2, HK2, NID1, |
| | | | | | | | SFRP1, TG, TRPC1, VEGFA |
| LET-7A-5P | | mature | | 1.25 | bias | 0.0014 | ACTA2, BCL7A, COL27A1, COL3A1, |
| (AND | | microRNA | | | | | COL5A2, FN1, ITGB3, NID1, NQO1, SCD, |
| OTHER | | | | | | | SMAD2 |
| MIRNAS | | | | | | | |
| W/SEED | | | | | | | |
| GAGGUAG) | | | | | | | |
| NFKB | | complex | | −1.386 | | 0.00144 | A2M, AICDA, BCL11A, CCR2, CD69, |
| (COMPLEX) | | | | | | | CEBPB, CLCF1, CREM, CXCL10, DAXX, |
| | | | | | | | FCER2, FN1, GADD45A, IGKC, IL4, IL5, |
| | | | | | | | IL9, IRF4, MIR-29, NFKBIZ, SPP1, TGM2, |
| | | | | | | | TSLP, VEGFA |
| ERBB3 | −1.599 | kinase | | −1 | | 0.00148 | ACTA2, FN1, GAS5, NID1, PHGDH, SFRP1, |
| | | | | | | | TGM2, VEGFA |
| CIP2A | 0.131 | other | | 0 | | 0.0016 | GADD45A, H2BC12, HK2, NPTX1, |
| | | | | | | | PDGFRB, PRG4 |
| PDGFR | | group | | | | 0.00163 | ACTA2, AREG, FN1 |
| CCL21 | | cytokine | | | | 0.00163 | CXCL10, IL4, IL5 |
| SERPINB5 | | other | | | | 0.00163 | IL4, IL5, VEGFA |
| PI3K | | complex | | −1.952 | | 0.00166 | ATF4, CD2, CEBPB, CXCL10, FN1, HK2, |
| (COMPLEX) | | | | | | | IL4, NQO1, NR4A2, RPS6, SELL, SPP1, |
| | | | | | | | TGM2, VEGFA |
| ACE | −1.023 | peptidase | | 1 | | 0.00175 | CEBPB, IL4, IL5, IL5RA |
| TFPI2 | | other | | 1.131 | | 0.00175 | ACTA2, AREG, CXCL10, HSPA2 |
| SCGB1A1 | | cytokine | | 1.993 | | 0.00175 | FN1, IL4, IL5, SELL |
| HGF | −0.131 | growth | | −0.973 | | 0.00177 | A2M, ACTA2, CLCF1, COL3A1, EMP1, |
| | | factor | | | | | FN1, GRB10, HK2, IL4, IL5, IL5RA, |
| | | | | | | | INSIG1, MET, NFKBIZ, NR4A2, PHLDA1, |
| | | | | | | | SFRP1, SPP1, TNFSF15, VEGFA |
| AMBRA1 | −0.003 | other | | 1.808 | | 0.00182 | COL27A1, COL3A1, COL5A2, FN1, ITGB3 |
| PPARD | −0.036 | ligand- | | −0.089 | | 0.00188 | ACTA2, ALDH2, CEBPB, DHRS2, FABP3, |
| | | dependent | | | | | FN1, LIPG, MPEG1, MYLIP, NR4A2, |
| | | nuclear | | | | | PGM5, SCD, VEGFA, VLDLR |
| | | receptor | | | | | |
| SMAD4 | −0.097 | transcription | | −0.02 | | 0.00188 | AREG, CD79A, DAXX, FN1, GADD45A, |
| | | regulator | | | | | IL3, IL5, IL9, JAG2, MET, PDGFRB, SCD, |
| | | | | | | | SMAD2, VEGFA |
| IL7 | 0.684 | cytokine | | −0.288 | | 0.00196 | BLNK, CD2, CD69, CD8A, FN1, IL4, IL5, |
| | | | | | | | RAG1, SELL |
| GPD1 | 0.348 | enzyme | | | | 0.00196 | CXCL13, GADD45A, NQO1, NUDT7, |
| | | | | | | | PSAT1, TTC39C, VLDLR |

### Example 2—Exemplary Sequences

Exemplary Cas9 polypeptide sequence
(SEQ ID NO: 3)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

### Example 3—Treating Cancer

A human having cancer is administered CAR T cells having a reduced level of an Axl polypeptide (e.g., Axl KO CAR T cells). The administered CAR T cells having a reduced level of an Axl polypeptide (e.g., Axl KO CAR T cells) can target (e.g., target and destroy) cancer cells (e.g., cancer cells expressing a tumor antigen targeted by the CAR T cells) within a mammal.

### Example 4—Treating Cancer

T cells are obtained from a mammal having cancer and are engineered to be CAR T cells having a reduced level of an Axl polypeptide (e.g., Axl KO CAR T cells). The CAR T cells having a reduced level of an Axl polypeptide (e.g., Axl KO CAR T cells) are administered back to the human. The administered CAR T cells having a reduced level of an Axl polypeptide (e.g., Axl KO CAR T cells) can target (e.g., target and destroy) cancer cells (e.g., cancer cells expressing a tumor antigen targeted by the CAR T cells) within a mammal.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of generating a T cell having a reduced level of an Axl polypeptide, wherein said method comprises:
(a) introducing, into a T cell, a nucleic acid encoding a Cas nuclease and a guide RNA, and
(b) culturing said T cell under conditions in which said Cas nuclease and said guide RNA are expressed and said Cas nuclease cleaves genomic DNA within said T cell at a nucleotide sequence targeted by said guide RNA, wherein said nucleic acid encoding said guide RNA comprises the sequence set forth in SEQ ID NO: 1 or the sequence set forth in any one of the sequence identifiers set forth in TABLE 1.

2-3. (canceled)

4. The method of claim 1, wherein said T cell expresses a reduced level of said Axl polypeptide as compared to a comparable T cell lacking said disruption.

5. The method of claim 1, wherein said method further comprises introducing, into said T cell, a nucleic acid encoding a chimeric antigen receptor.

6-10. (canceled)

11. A T cell comprising:
(a) a reduced level of an Axl polypeptide, and
(b) nucleic acid encoding a chimeric antigen receptor,
wherein said T cell expresses said chimeric antigen receptor, and
wherein said T cell was generated by a method that comprises introducing, into said T cell or a precursor thereof, a Cas nuclease and a nucleotide sequence encoding a guide RNA, said nucleotide sequence encoding said guide RNA comprises the sequence set forth in SEQ ID NO:1 or the sequence set forth in any one of the sequence identifiers set forth in TABLE 1, such that said Cas nuclease cleaves genomic DNA within said T cell at a nucleotide sequence targeted by said guide RNA.

12-13. (canceled)

14. The T cell of claim 11, wherein said T cell expresses a reduced level of said Axl polypeptide as compared to a comparable T cell lacking said disruption.

15-17. (canceled)

18. A method for treating a mammal having cancer, wherein said method comprises administering to said mammal a composition comprising the T cell comprising:
(a) a reduced level of an Axl polypeptide, and
(b) nucleic acid encoding a chimeric antigen receptor,
wherein said T cell expresses said chimeric antigen receptor.

19. The method of claim 18, wherein said mammal is a human.

20. The method of claim 18, wherein said composition comprises from about 0.1×10⁶ to about 10×10⁶ of said T cells.

21. The method of claim 18, wherein said cancer is selected from diffuse large B cell lymphoma (DLBCL), Hodgkin's lymphomas, non-Hodgkin lymphomas, acute lymphoblastic leukemia (ALL), chronic lymphocytic leukemia (CLL), acute myeloid leukemia (AML), germ cell tumors, hepatocellular carcinoma, bowel cancer, lung cancer, breast cancer, ovarian cancer, melanoma, epithelial tumors, brain cancers, multiple myelomas, sarcomas, bone cancers, pancreatic cancers, liver cancers, myeloid neoplasms, prostate cancers, and thyroid cancers.

22. The method of claim 18, wherein said T cell was generated by a method that comprises introducing, into said T cell or a precursor thereof, a Cas nuclease and a nucleotide sequence encoding a guide RNA, said nucleotide sequence encoding said guide RNA comprises the sequence set forth in SEQ ID NO: 1 or the sequence set forth in any one of the sequence identifiers set forth in TABLE 1, such that said Cas nuclease cleaves genomic DNA within said T cell at a nucleotide sequence targeted by said guide RNA.

23. The method of claim 18, wherein said T cell was generated by a method that comprises:
(i) introducing, into said T cell or a precursor thereof, a transcription activator-like effector (TALE) nuclease, wherein said TALE nuclease comprises a first monomer and a second monomer, and
(ii) culturing said T cell under conditions in which said first TALE nuclease monomer and said second TALE nuclease monomer are expressed within said T cell, wherein said first TALE nuclease monomer binds to a first target sequence in genomic DNA of said T cell, wherein said second TALE nuclease monomer binds to a second target sequence in said genomic DNA, and wherein said first and second TALE nuclease monomers dimerize and cleave said genomic DNA of said T cell, wherein said first and second target sequences comprise a pair of nucleotide sequences set forth in the sequence identifier pairs that are set forth in TABLE 2.

24. (canceled)

25. A method of generating a T cell having a reduced level of an Axl polypeptide, wherein said method comprises:
(a) introducing, into a T cell, nucleic acid encoding a transcription activator-like effector (TALE) nuclease, wherein said TALE nuclease comprises a first monomer and a second monomer, and
(b) culturing said T cell under conditions in which said first TALE nuclease monomer and said second TALE nuclease monomer are expressed within said T cell, wherein said first TALE nuclease monomer binds to a first target sequence in genomic DNA of said T cell, wherein said second TALE nuclease monomer binds to a second target sequence in said genomic DNA, and wherein said first and second TALE nuclease monomers dimerize and cleave said genomic DNA of said T cell, wherein said first and second target sequences comprise a pair of nucleotide sequences set forth in the sequence identifier pairs that are set forth in TABLE 2.

26-27. (canceled)

28. The method of claim 25, wherein said T cell expresses a reduced level of said Axl polypeptide as compared to a comparable T cell lacking said disruption.

29. The method of claim 25, wherein said method further comprises introducing, into said T cell, a nucleic acid encoding a chimeric antigen receptor.

30-34. (canceled)

35. A T cell comprising:
(a) a reduced level of an Axl polypeptide, and
(b) nucleic acid encoding a chimeric antigen receptor,
wherein said T cell expresses said chimeric antigen receptor, and
wherein said T cell was generated by a method that comprises:
(i) introducing, into said T cell or a precursor thereof, a transcription activator-like effector (TALE) nuclease, wherein said TALE nuclease comprises a first monomer and a second monomer, and
(ii) culturing said T cell under conditions in which said first TALE nuclease monomer and said second TALE nuclease monomer are expressed within said T cell, wherein said first TALE nuclease monomer binds to a first target sequence in genomic DNA of said T cell, wherein said second TALE nuclease monomer binds to a second target sequence in said genomic DNA, and wherein said first and second TALE nuclease monomers dimerize and cleave said genomic DNA of said T cell, wherein said first and second target sequences comprise a pair of nucleotide sequences set forth in the sequence identifier pairs that are set forth in TABLE 2.

36-37. (canceled)

38. The T cell of claim 35, wherein said T cell expresses a reduced level of said Axl polypeptide as compared to a comparable T cell lacking said disruption.

39. The T cell of claim 35, wherein said chimeric antigen receptor targets a tumor-associated antigen.

40. The T cell of claim 39, wherein said tumor-associated antigen is CD19.

41. The T cell of claim 35, wherein said T cell is obtained from a human.

42-48. (canceled)


SEQUENCE LISTING (29 sequences)

SEQ ID NO: 1 - DNA, 20 bp, synthetic construct
  source 1..20: mol_type = other DNA
ttcggtgtcagctccaggtt

SEQ ID NO: 2 - DNA, 20 bp, synthetic construct
  source 1..20: mol_type = other DNA
aacctggagctgacaccgaa

SEQ ID NO: 3 - protein, 1368 aa, Streptococcus pyogenes [sequence omitted]

SEQ ID NO: 4 - DNA, 20 bp, synthetic construct
  source 1..20: mol_type = other DNA
caccccttatcacatccgcg

SEQ ID NO: 5 - DNA, 20 bp, synthetic construct
  source 1..20: mol_type = other DNA
accgatgagcctcatgacgt

SEQ ID NO: 6 - DNA, 20 bp, synthetic construct
  source 1..20: mol_type = other DNA
atgccacgcggatgtgataa

SEQ ID NO: 7 - DNA, 20 bp, synthetic construct
  source 1..20: mol_type = other DNA
cgcgtagcactaatgttctc

SEQ ID NO: 8 - DNA, 20 bp, synthetic construct
  source 1..20: mol_type = other DNA
gactgtaggacttgcgcacg

SEQ ID NO: 9 - DNA, 20 bp, synthetic construct
  source 1..20: mol_type = other DNA
gttctccacgcccggatatg

SEQ ID NO: 10 - DNA, 20 bp, synthetic construct
  source 1..20: mol_type = other DNA
ttgacatagaggatttcgtc

SEQ ID NO: 11 - DNA, 20 bp, synthetic construct
  source 1..20: mol_type = other DNA
cgccagccggtaccctaaca

SEQ ID NO: 12 - DNA, 20 bp, synthetic construct
  source 1..20: mol_type = other DNA
gcatcttggcgatacgtccc

SEQ ID NO: 13 - DNA, 20 bp, synthetic construct
  source 1..20: mol_type = other DNA
tcttggcgatacgtccctgg

SEQ ID NO: 14 - DNA, 20 bp, synthetic construct
  source 1..20: mol_type = other DNA
tgcgcaagtcctacagtcgt

SEQ ID NO: 15 - DNA, 20 bp, synthetic construct
  source 1..20: mol_type = other DNA
acattagtgctacgcggaat

SEQ ID NO: 16 - DNA, 20 bp, synthetic construct
  source 1..20: mol_type = other DNA
aatggggactactaccgcca

SEQ ID NO: 17 - DNA, 20 bp, synthetic construct
  source 1..20: mol_type = other DNA
gagattcatacaccgggacc

SEQ ID NO: 18 - DNA, 20 bp, synthetic construct
  source 1..20: mol_type = other DNA
cgaaagaaggagacccgtta

SEQ ID NO: 19 - DNA, 20 bp, synthetic construct
  source 1..20: mol_type = other DNA
ccttgtccaccggcgaaaga

SEQ ID NO: 20 - DNA, 19 bp, Homo sapiens
  source 1..19: mol_type = genomic DNA
ggctgtgctgtcagacgat

SEQ ID NO: 21 - DNA, 22 bp, Homo sapiens
  source 1..22: mol_type = genomic DNA
ggcactgatataagcaaggtcc

SEQ ID NO: 22 - DNA, 20 bp, Homo sapiens
  source 1..20: mol_type = genomic DNA
ctctcctcaggctgtgctgt

SEQ ID NO: 23 - DNA, 22 bp, Homo sapiens
  source 1..22: mol_type = genomic DNA
ggcactgatataagcaaggtcc

SEQ ID NO: 24 - DNA, 19 bp, Homo sapiens
  source 1..19: mol_type = genomic DNA
ctgtcagacgatgggatgg

SEQ ID NO: 25 - DNA, 22 bp, Homo sapiens
  source 1..22: mol_type = genomic DNA
ggcactgatataagcaaggtcc

SEQ ID NO: 26 - DNA, 20 bp, Homo sapiens
  source 1..20: mol_type = genomic DNA
aggctgtgctgtcagacgat

SEQ ID NO: 27 - DNA, 22 bp, Homo sapiens
  source 1..22: mol_type = genomic DNA
ggcactgatataagcaaggtcc

SEQ ID NO: 28 - DNA, 20 bp, Homo sapiens
  source 1..20: mol_type = genomic DNA
gtgctgtcagacgatgggat

SEQ ID NO: 29 - DNA, 22 bp, Homo sapiens
  source 1..22: mol_type = genomic DNA
ggcactgatataagcaaggtcc